(19) United States
(12) Patent Application Publication
(10) Pub. No.: US 2015/0152455 A1
(43) Pub. Date: Jun. 4, 2015

(54) Polypeptides Having Berberine Bridge Enzyme Like Activity and Polynucleotides Encoding Same

(71) Applicant: Novozymes A/S, Bagsvaerd (DK)

(72) Inventors: Kirk Matthew Schnorr, Holte (DK); Junxin Duan, Beijing (CN); Lars Kiemer, Ballerup (DK); Hong Zhi Huang, Beijing (CN)

(21) Appl. No.: 14/375,402

(22) PCT Filed: Jan. 29, 2013
(86) PCT No.: PCT/CN2013/071080

§ 371 Date: Jul. 29, 2014

(30) Foreign Application Priority Data
Jan. 30, 2012 (CN) PCTCN2012070775
May 22, 2012 (CN) PCTCN2012075892

(51) Int. Cl. C12P 19/14; C12P 19/02; C12N 9/02

(52) U.S. Cl. CPC C12P 19/14; C12N 9/0004; C12P 19/02; C12Y 121/03003; C12P 2201/00; C12P 2203/00

(57) ABSTRACT

Provided are isolated polypeptides having Berberine Bridge Enzyme Like activity, catalytic domains, FAD binding domains and polynucleotides encoding the polypeptides, catalytic domains or FAD binding domains. The nucleic acid constructs, vectors, and host cells comprising the polynucleotides as well as methods of producing and using the polypeptides, catalytic domains or FAD binding domains are also provided.

### REFERENCE TO A SEQUENCE LISTING

This application contains a Sequence Listing in computer readable form, which is incorporated herein by reference.

### BACKGROUND OF THE INVENTION

1. Field of the Invention

The present invention relates to polypeptides having Berberine Bridge Enzyme Like (BBEL) activity, catalytic domains, and flavin adenine dinucleotide (FAD) binding domains, and polynucleotides encoding the polypeptides, catalytic domains, and FAD binding domains. The invention also relates to nucleic acid constructs, vectors, and host cells comprising the polynucleotides as well as methods of producing and using the polypeptides, catalytic domains, and FAD binding domains.

2. Description of the Related Art

Previously described involvement of berberine bridge enzymes includes W02000/53737-A2 in which a BBEL enzyme forms part of antibiotic gene cluster for mitomycin production from Streptomyces. CN101157929-A describes a gene cluster with BBE activity for saframycin biosynthesis. W02002/68643-A1 describes a BBEL enzyme of fungal origin and discusses usefulness of the oxidase in a molt hormone-inactivation system.

Cellulose is a polymer of the simple sugar glucose covalently linked by beta-1,4-bonds. Many microorganisms produce enzymes that hydrolyze beta-linked glucans. These enzymes include endoglucanases, cellobiohydrolases, and beta-glucosidases. Endoglucanases digest the cellulose polymer at random locations, opening it to attack by cellobiohydrolases. Cellobiohydrolases sequentially release molecules of cellobiose from the ends of the cellulose polymer. Cellobiose is a water-soluble beta-1,4-linked dimer of glucose. Beta-glucosidases hydrolyze cellobiose to glucose.

The conversion of lignocellulosic feedstocks into ethanol has the advantages of the ready availability of large amounts of feedstock, the desirability of avoiding burning or land filling the materials, and the cleanliness of the ethanol fuel. Wood, agricultural residues, herbaceous crops, and municipal solid wastes have been considered as feedstocks for ethanol production. These materials primarily consist of cellulose, hemicellulose, and lignin. Once the lignocellulose is converted to fermentable sugars, e.g., glucose, the fermentable sugars are easily fermented by yeast into ethanol.

The present invention provides polypeptides having Berberine Bridge Enzyme Like activity and polynucleotides encoding the polypeptides. In particular the present invention relates to berberine bridge ring forming enzymes for boosting biomass hydrolysis. In a related aspect, the present invention provides isolated polypeptides having cellulolytic enhancing activity and isolated nucleic acid sequences encoding the polypeptides to improve the conversion of cellulosic feedstocks at higher temperatures.

### SUMMARY OF THE INVENTION

The present invention relates to an enzyme composition comprising a cellulolytic preparation and a Berberine Bridge Enzyme Like (BBEL) enzyme, which optionally is derived from Penicillium pinophilum or Aspergillus aculeatus.

Further, the present invention relates to a method of hydrolyzing cellulosic material, comprising subjecting the cellulosic material to a cellulolytic preparation and a Berberine Bridge Enzyme Like (BBEL) enzymes.

Present invention also relates to a process of producing a fermentation product from cellulosic material, comprising:

hydrolyzing said cellulosic material by subjecting the material to an enzyme composition comprising a cellulolytic preparation and a Berberine Bridge Enzyme Like (BBEL) enzyme or by a method comprising subjecting the cellulosic material to a cellulolytic preparation and a Berberine Bridge Enzyme Like (BBEL) enzyme;

fermenting using a fermenting organism; and optionally recovering the fermentation product.

The present invention also relates to isolated polypeptides having Berberine Bridge Enzyme Like (BBEL) activity selected from the group consisting of:

(a) a polypeptide having at least 95% sequence identity to the mature polypeptide of SEQ ID NO: 2, at least 90% sequence identity to the mature polypeptide of SEQ ID NO: 4, at least 80% sequence identity to the mature polypeptide of any of SEQ ID NO: 6, SEQ ID NO: 8 or SEQ ID NO: 10, at least 75% sequence identity to the mature polypeptide of any of SEQ ID NO: 12 or SEQ ID NO:14, or at least 60% sequence identity to the mature polypeptide of SEQ ID NO: 16;

(b) a polypeptide encoded by a polynucleotide that hybridizes under medium, medium-high, high, or very high stringency conditions with
(i) the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15,
(ii) the cDNA sequence thereof or
(iii) the full-length complement of (i) or (ii);

(c) a polypeptide encoded by a polynucleotide having at least 95% sequence identity to the mature polypeptide coding sequence of SEQ ID NO: 1, at least 90% sequence identity to the mature polypeptide coding sequence of SEQ ID NO: 3, at least 80% sequence identity to the mature polypeptide coding sequence of any of SEQ ID NO: 5, SEQ ID NO: 7 or SEQ ID NO: 9, at least 75% sequence identity to the mature polypeptide coding sequence of SEQ ID NO: 11 or SEQ ID NO:13, or at least 60% sequence identity to the mature polypeptide coding sequence of SEQ ID NO: 15

(d) a variant of the mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16, comprising a substitution, deletion, and/or insertion at one or more (e.g., several) positions; and

(e) a fragment of the polypeptide of (a), (b), (c), or (d) that has Berberine Bridge Enzyme Like (BBEL) activity.

The present invention also relates to isolated polypeptides comprising a catalytic domain selected from the group consisting of:

(a) a catalytic domain having at least 60% sequence identity to amino acids 428 to 472 of SEQ ID NO: 2, 502 to 541 of SEQ ID NO: 4, 463 to 503 of SEQ ID NO: 6, 431 to 473 of SEQ ID NO: 8, 572 to 615 of SEQ ID NO: 10, 517 to 561 of SEQ ID NO: 12, 546 to 581 of SEQ ID NO: 14 or 448 to 484 of SEQ ID NO: 16;

(b) a catalytic domain encoded by a polynucleotide that hybridizes under medium, medium-high, high, or very high stringency conditions with

(i) nucleotides 1370 to 1504 of SEQ ID NO: 1, nucleotides 1615 to 1629 and/or 1680 to 1784 of SEQ ID NO: 3, nucleotides 1498 to 1620 of SEQ ID NO: 5, nucleotides 1575 to 1703 of SEQ ID NO:7, nucleotides 1834 to 1965 of SEQ ID NO: 9, nucleotides 1649 to 1783 of SEQ ID NO: 11, nucleotides 1697 to 1804 of SEQ ID NO: 13 an or nucleotides 1400 to 1401 and/or 1511 to 1619 of SEQ ID NO:15;

(ii) the cDNA sequence thereof, or

(iii) the full-length complement of (i) or (ii);

(c) a catalytic domain encoded by a polynucleotide having at least 90% sequence identity to nucleotides 1370 to 1504 of SEQ ID NO: 1, nucleotides 1615 to 1629 and/or 1680 to 1784 of SEQ ID NO: 3, nucleotides 1498 to 1620 of SEQ ID NO: 5, nucleotides 1575 to 1703 of SEQ ID NO:7, nucleotides 1834 to 1965 of SEQ ID NO: 9, nucleotides 1649 to 1783 of SEQ ID NO: 11, nucleotides 1697 to 1804 of SEQ ID NO: 13 or nucleotides 1400 to 1401 and/or 1511 to 1619 of SEQ ID NO:15, or the cDNA sequence thereof;

(d) a variant of amino acids 428 to 472 of SEQ ID NO: 2, 502 to 541 of SEQ ID NO: 4, 463 to 503 of SEQ ID NO: 6, 431 to 473 of SEQ ID NO: 8, 572 to 615 of SEQ ID NO: 10, 517 to 561 of SEQ ID NO: 12, 546 to 581 of SEQ ID NO: 14 or 448 to 484 of SEQ ID NO: 16,

comprising a substitution, deletion, and/or insertion at one or more (e.g., several) positions; and

(e) a fragment of the catalytic domain of (a), (b), (c), or (d) that has Berberine Bridge Enzyme Like (BBEL) activity.

The present invention also relates to isolated polypeptides comprising a FAD binding domain selected from the group consisting of:

(a) a FAD binding domain having at least 60% sequence identity to amino acids 48 to 186 of SEQ ID NO: 2, 122 to 256 of SEQ ID NO: 4, 80 to 213 of SEQ ID NO: 6, 54 to 188 of SEQ ID NO: 8, 179 to 319 of SEQ ID NO: 10, 128 to 265 of SEQ ID NO: 12, 119 to 261 of SEQ ID NO: 14 or 63 to 199 of SEQ ID NO: 16;

(b) a FAD binding domain encoded by a polynucleotide that hybridizes under medium, medium-high, high, or very high stringency conditions with

(i) nucleotides 142 to 259 and/or 348 to 646 of SEQ ID NO: 1, nucleotides 424 to 509 and/or 561 to 879 of SEQ ID NO: 3, nucleotides 238 to 279 and/or 338 to 697 of SEQ ID NO: 5, nucleotides 160 to 201, 266 to 482 and/or 548 to 693 of SEQ ID NO:7, nucleotides 590 to 1012 of SEQ ID NO: 9, nucleotides 433 to 524 and/or 574 to 895 of SEQ ID NO: 11, nucleotides 355 to 470 and/or 532 to 844 of SEQ ID NO: 13 or nucleotides 187 to 228 and/or 287 to 655 of SEQ ID NO:15,

(ii) the cDNA sequence thereof, or

(iii) the full-length complement of (i) or (ii);

(c) a FAD binding domain encoded by a polynucleotide having at least 90% sequence identity to nucleotides 142 to 259 and/or 348 to 646 of SEQ ID NO: 1, nucleotides 424 to 509 and/or 561 to 879 of SEQ ID NO: 3, nucleotides 238 to 279 and/or 338 to 697 of SEQ ID NO: 5, nucleotides 160 to 201, 266 to 482 and/or 548 to 693 of SEQ ID NO:7, nucleotides 590 to 1012 of SEQ ID NO: 9, nucleotides 433 to 524 and/or 574 to 895 of SEQ ID NO: 11, nucleotides 355 to 470 and/or 532 to 844 of SEQ ID NO: 13 or nucleotides 187 to 228 and/or 287 to 655 of SEQ ID NO:15, or the cDNA sequence thereof;

(d) a variant of amino acids 48 to 186 of SEQ ID NO: 2, 122 to 256 of SEQ ID NO: 4, 80 to 213 of SEQ ID NO: 6, 54 to 188 of SEQ ID NO: 8, 179 to 319 of SEQ ID NO: 10, 128 to 265 of SEQ ID NO: 12, 119 to 261 of SEQ ID NO: 14 or 63 to 199 of SEQ ID NO: 16 comprising a substitution, deletion, and/or insertion at one or more (e.g., several) positions; and

(e) a fragment of the FAD binding domain of (a), (b), (c), or (d) that has binding activity.

The present invention also relates to an isolated polypeptide comprising a FAD binding domain operably linked to a catalytic domain, wherein the binding domain is selected from the group consisting of:

(a) a FAD binding domain having at least 60% sequence identity to amino acids 48 to 186 of SEQ ID NO: 2, 122 to 256 of SEQ ID NO: 4, 80 to 213 of SEQ ID NO: 6, 54 to 188 of SEQ ID NO: 8, 179 to 319 of SEQ ID NO: 10, 128 to 265 of SEQ ID NO: 12, 119 to 261 of SEQ ID NO: 14 or 63 to 199 of SEQ ID NO: 16;

(b) a FAD binding domain encoded by a polynucleotide that hybridizes under medium, medium-high, high, or very high stringency conditions with

(i) nucleotides 142 to 259 and/or 348 to 646 of SEQ ID NO: 1, nucleotides 424 to 509 and/or 561 to 879 of SEQ ID NO: 3, nucleotides 238 to 279 and/or 338 to 697 of SEQ ID NO: 5, nucleotides 160 to 201, 266 to 482 and/or 548 to 693 of SEQ ID NO:7, nucleotides 590 to 1012 of SEQ ID NO: 9, nucleotides 433 to 524 and/or 574 to 895 of SEQ ID NO: 11, nucleotides 355 to 470 and/or 532 to 844 of SEQ ID NO: 13 and/or nucleotides 187 to 228 and/or 287 to 655 of SEQ ID NO:15,

(ii) the cDNA sequence thereof, or

(iii) the full-length complement of (i) or (ii);

(c) a FAD binding domain encoded by a polynucleotide having at least 90% sequence identity to nucleotides 142 to 259 and/or 348 to 646 of SEQ ID NO: 1, nucleotides 424 to 509 and/or 561 to 879 of SEQ ID NO: 3, nucleotides 238 to 279 and/or 338 to 697 of SEQ ID NO: 5, nucleotides 160 to 201, 266 to 482 and/or 548 to 693 of SEQ ID NO:7, nucleotides 590 to 1012 of SEQ ID NO: 9, nucleotides 433 to 524 and/or 574 to 895 of SEQ ID NO: 11, nucleotides 355 to 470 and/or 532 to 844 of SEQ ID NO: 13 and/or nucleotides 187 to 228 and/or 287 to 655 of SEQ ID NO:15, or the cDNA sequence thereof;

(d) a variant of amino acids 48 to 186 of SEQ ID NO: 2, 122 to 256 of SEQ ID NO: 4, 80 to 213 of SEQ ID NO: 6, 54 to 188 of SEQ ID NO: 8, 179 to 319 of SEQ ID NO: 10, 128 to 265 of SEQ ID NO: 12, 119 to 261 of SEQ ID NO: 14 or 63 to 199 of SEQ ID NO: 16 comprising a substitution, deletion, and/or insertion at one or more (e.g., several) positions; and

(e) a fragment of the FAD binding domain of (a), (b), (c), or (d) that has binding activity.

Additionally, the present invention relates to an isolated polynucleotide encoding a signal peptide comprising or consisting of amino acids 1 to 20 of SEQ ID NO: 4, amino acids 1 to 20 of SEQ ID NO: 6, amino acids 1 to 13 of SEQ ID NO: 8, amino acids 1 to 20 of SEQ ID NO: 10, amino acids 1 to 19 of SEQ ID NO: 12, amino acids 1 to 18 of SEQ ID NO: 14 or amino acids 1 to 20 of SEQ ID NO: 16 each of which may be operably linked to a gene encoding a protein; nucleic acid constructs, expression vectors, and recombinant host cells comprising the polynucleotides; and methods of producing a protein.

The present invention also relates to isolated polynucleotides encoding the polypeptides of the present invention; nucleic acid constructs; recombinant expression vectors; recombinant host cells comprising the polynucleotides; and methods of producing the polypeptides.

The present invention also relates to methods of boosting hydrolysis of biomass.

### BRIEF DESCRIPTION OF THE FIGURES

FIG. 1 shows a comparative dose-response trial on varying amounts of BBEL enzyme tested on an enzymatic background of a cellulolytic enzyme composition derived from Trichoderma reesei, further comprising: an Aspergillus fumigatus cellobiohydrolase I; an Aspergillus fumigatus cellobiohydrolase II; an Aspergillus fumigatus beta-glucosidase or variant thereof; and a Penicillium sp. GH61 polypeptide having cellulolytic enhancing activity; or homologs thereof after 3 days hydrolysis on 12.6% TS, NREL unwashed PCS.

The control: 5 mg cellulolytic enzyme composition (CEC)/g cellulose was added in the control sample. For samples containing BBEL, 10 ug means 10 ug BBEL/g cellulose was added on top of 5 mg CEC/g cellulose, etc.

Sample Legend:

BBE3834=P23WGQ from P. pinophilum, represented by SEQ ID NO:3 and SEQ ID NO:4 herein.

BBE3824=P2416F from A. aculeatus, represented by SEQ ID NO:7 and SEQ ID NO:8 herein.

BBE3887=P244GZ from A. aculeatus, represented by SEQ ID NO:9 and SEQ ID NO:10 herein.

BBE3888=P23WH5 from P. pinophilum, represented by SEQ ID NO:1 and SEQ ID NO:2 herein.

BBE3889=P244GP from A. aculeatus, represented by SEQ ID NO:5 and SEQ ID NO:6 herein.

FIG. 2 shows an identity matrix in which all sequences of present invention are scored relatively to each other. The matrix is calculated using the Needleman Wunsch method described elsewhere in this document. Substitution matrix: Blosum 62, Program: Needleman Wunsch, Gap initiation penalty: 10.0, Gap extension penalty: 0.5, Gapless Identity Matrix.

FIG. 3 shows a Clustal W alignment of SEQ ID NO:2 to SEQ ID NO: 16. From the alignment, the skilled person will readily locate regions with high variability and regions or amino acids that are conserved between the sequences. Amino acid deletions, substitutions or insertions in regions with high variability are less likely to influence the function of the peptide than amino acid deletions, substitutions or insertion in regions with low variability or high level conservation.

### DEFINITIONS

Berberine Bridge Enzyme (BBE):

The defining molecule for this class is the Berberine Bridge Enzyme (BBE) (EC 1.21.3.3) from California poppy. BBE is an FAD containing enzyme responsible for making the precusor to insoquinoline alkaloids in plants. The holoenzyme catalyzes the ring closure of reticuline to scoulerine via the transformation of the N-methyl group of S-reticuline into the berberine bridge carbon (C-8) of S-scoulerine see figure:

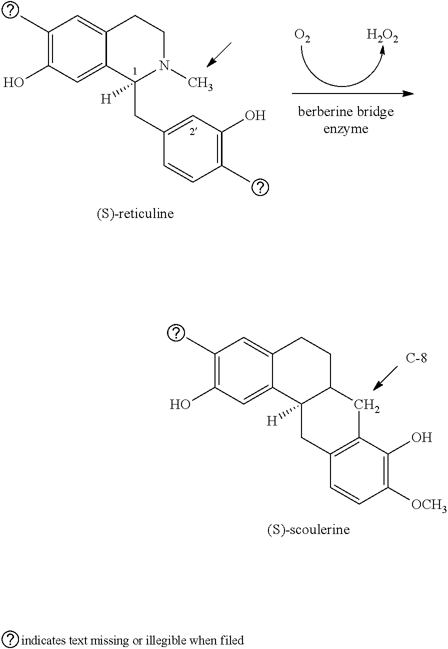

Winkler A et al. J. Biol. Chem. 2006; 281:21276-21285

The term “berberine bridge enzyme” means a FAD containing enzyme with BBEL activity (EC 1.21.3.3) optionally acting on (S)-reticuline and related compounds, converting the N-methyl group into the methylene bridge (′berberine bridge′) of (S)-tetrahydroprotoberberines. Examples of other accepted names of BBE's are: reticuline oxidase, (S)-reticuline, berberine bridge enzyme, or tetrahydroprotoberberine synthase.

Berberine Bridge Enzyme Like (BBEL) Enzymes

By BBEL enzymes is meant a peptide sequence longer than 350 amino acids containing 1) the Pfam domain PF01565 (FAD binding 4), 2) the Pfam domain PF08031 (berberine bridge enzyme (BBE)). The Pfam domains are detected with default detection thresholds. The PF08031 domain is always in the C-terminal part of the peptide sequence relative to the PF01565 domain, and they are separated by a stretch of up to 500 amino acids of unknown function. BBEL enzymes oxidize their substrates and thereby generate hydrogen peroxide.

For purposes of the present invention, Berberine Bridge Enzyme Like (BBEL) enzymatic activity or cellulolytic enhancing activity effect is determined according to the procedure described in Example 6. In one aspect, the polypeptides of the present invention have at least 20%, e.g., at least 40%, at least 50%, at least 60%, at least 70%, at least 80%, at least 90%, at least 95%, or at least 100% of the berberine bridge enzyme activity of the mature polypeptide of SEQ ID NO: 2, SEQ ID NO: 4 SEQ ID NO: 6, SEQ ID NO: 8, SEQ ID NO: 10, SEQ ID NO: 12, SEQ ID NO: 14, SEQ ID NO: 16.

Allelic variant: The term “allelic variant” means any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

Binding domain: The term “FAD binding domain” or “flavin adenine dinucleotide binding domain” means the region of an enzyme that mediates binding of FAD or flavin adenine dinucleotide. The FAD binding domain is typically found either at the N-terminal or at the C-terminal extremity of an berberine bridge enzyme.

Catalytic domain: The term “catalytic domain” means the region of an enzyme containing the catalytic machinery of the enzyme. In particular, the term “catalytic domain” relates to the Berberine Bridge Enzyme Like (BBEL) domain.

cDNA: The term “cDNA” means a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

Cellulolytic preparation: The term cellulolytic preparation means a composition of enzymatic activities capable of converting cellulosic or lignocellulosic material into fermentable sugars. A cellulolytic preparation may be isolated from a microorganism. A cellulolytic preparation can be composed of several enzymatic activities, these can be selected from the group consisting of beta-glucosidase, GH61, xylanase, beta-xylosidase, cellobiohydrolase I, cellobiohydrolase II.

Coding sequence: The term “coding sequence” means a polynucleotide, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

Control sequences: The term “control sequences” means nucleic acid sequences necessary for expression of a polynucleotide encoding a mature polypeptide of the present invention. Each control sequence may be native (i.e., from the same gene) or foreign (i.e., from a different gene) to the polynucleotide encoding the polypeptide or native or foreign to each other. Such control sequences include, but are not limited to, a leader, polyadenylation sequence, propeptide sequence, promoter, signal peptide sequence, and transcription terminator. At a minimum, the control sequences include a promoter, and transcriptional and translational stop signals. The control sequences may be provided with linkers for the purpose of introducing specific restriction sites facilitating ligation of the control sequences with the coding region of the polynucleotide encoding a polypeptide.

Expression: The term “expression” includes any step involved in the production of a polypeptide including, but not limited to, transcription, post-transcriptional modification, translation, post-translational modification, and secretion.

Expression vector: The term “expression vector” means a linear or circular DNA molecule that comprises a polynucleotide encoding a polypeptide and is operably linked to control sequences that provide for its expression.

Fragment: The term “fragment” means a polypeptide or a catalytic or FAD binding domain having one or more (e.g., several) amino acids absent from the amino and/or carboxyl terminus of a mature polypeptide or domain; wherein the fragment has Berberine Bridge Enzyme Like or FAD binding activity.

High stringency conditions: The term “high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 65° C.

Host cell: The term “host cell” means any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

Isolated: The term “isolated” means a substance in a form or environment that does not occur in nature. Non-limiting examples of isolated substances include (1) any non-naturally occurring substance, (2) any substance including, but not limited to, any enzyme, variant, nucleic acid, protein, peptide or cofactor, that is at least partially removed from one or more or all of the naturally occurring constituents with which it is associated in nature; (3) any substance modified by the hand of man relative to that substance found in nature; or (4) any substance modified by increasing the amount of the substance relative to other components with which it is naturally associated (e.g., multiple copies of a gene encoding the substance; use of a stronger promoter than the promoter naturally associated with the gene encoding the substance). An isolated substance may be present in a fermentation broth sample.

Low stringency conditions: The term “low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 50° C.

Mature polypeptide: The term “mature polypeptide” means a polypeptide in its final form following translation and any post-translational modifications, such as N-terminal processing, C-terminal truncation, glycosylation, phosphorylation, etc. In one aspect, the mature polypeptide is amino acids 29 to 471 of SEQ ID NO: 2, 21 to 561 of SEQ ID NO: 4, 21 to 508 of SEQ ID NO: 6, 14 to 474 of SEQ ID NO: 8, 21 to 638 of SEQ ID NO: 10, 20 to 577 of SEQ ID NO: 12, 19 to 607 of SEQ ID NO: 14 or 21 to 490 of SEQ ID NO: 16 based on the Pfam Program and database available from the Sanger Institute (Wellcome Trust Sanger Institute, Genome Research Limited, see The Pfam protein families database: R. D. Finn, J. Mistry, J. Tate, P. Coggill, A. Heger, J. E. Pollington, O. L. Gavin, P. Gunesekaran, G. Ceric, K. Forslund, L. Holm, E. L. Sonnhammer, S. R. Eddy, A. Bateman, Nucleic Acids Research (2010) Database Issue 38:D211-222) that predicts amino acids 1 to 20 of SEQ ID NO: 4, 1 to 20 of SEQ ID NO: 6, 1 to 13 of SEQ ID NO: 8, 1 to 20 of SEQ ID NO: 10, 1 to 19 of SEQ ID NO: 12, 1 to 18 of SEQ ID NO: 14 or 1 to 20 of SEQ ID NO: 16, are a signal peptide. It is known in the art that a host cell may produce a mixture of two of more different mature polypeptides (i.e., with a different C-terminal and/or N-terminal amino acid) expressed by the same polynucleotide.

Mature polypeptide coding sequence: The term “mature polypeptide coding sequence” means a polynucleotide that encodes a mature polypeptide having Berberine Bridge Enzyme Like activity. In one aspect, the mature polypeptide coding sequence is nucleotides 85 to 1513 of SEQ ID NO: 1, 61 to 1844 of SEQ ID NO: 3, 61 to 1635 of SEQ ID NO: 5, 40 to 1706 of SEQ ID NO: 7, 61 to 2034 of SEQ ID NO: 9, 58 to 1831 of SEQ ID NO: 11, 55 to 1882 of SEQ ID NO: 13 or 61 to 1637 of SEQ ID NO: 15, or the cDNA sequence thereof based on the Pfam Program and database available from the Sanger Institute (Wellcome Trust Sanger Institute, Genome Research Limited, see The Pfam protein families database: R. D. Finn, J. Mistry, J. Tate, P. Coggill, A. Heger, J. E. Pollington, O. L. Gavin, P. Gunesekaran, G. Ceric, K. Forslund, L. Holm, E. L. Sonnhammer, S. R. Eddy, A. Bateman, Nucleic Acids Research (2010) Database Issue 38:D211-222) that predicts nucleotides 1 to 20 of SEQ ID NO: 4, amino acids 1 to 20 of SEQ ID NO: 6, amino acids 1 to 13 of SEQ ID NO: 8, amino acids 1 to 20 of SEQ ID NO: 10, amino acids 1 to 19 of SEQ ID NO: 12, amino acids 1 to 18 of SEQ ID NO: 14 or amino acids 1 to 20 of SEQ ID NO: 16 to encode a signal peptide.

Medium stringency conditions: The term “medium stringency conditions” means for probes of at least 100 nucleotides in length, prehybridization and hybridization at 42° C. in 5×SSPE, 0.3% SDS, 200 micrograms/ml sheared and denatured salmon sperm DNA, and 35% formamide, following standard Southern blotting procedures for 12 to 24 hours. The carrier material is finally washed three times each for 15 minutes using 2×SSC, 0.2% SDS at 55° C.

Medium-high stringency conditions: The term “medium-high stringency conditions” means for probes of at least 100 nucleotides in length, prehybridization and hybridization at 42° C. in 5×SSPE, 0.3% SDS, 200 micrograms/ml sheared and denatured salmon sperm DNA, and either 35% formamide, following standard Southern blotting procedures for 12 to 24 hours. The carrier material is finally washed three times each for 15 minutes using 2×SSC, 0.2% SDS at 60° C.

Nucleic acid construct: The term “nucleic acid construct” means a nucleic acid molecule, either single- or double-stranded, which is isolated from a naturally occurring gene or is modified to contain segments of nucleic acids in a manner that would not otherwise exist in nature or which is synthetic, which comprises one or more control sequences.

Operably linked: The term “operably linked” means a configuration in which a control sequence is placed at an appropriate position relative to the coding sequence of a polynucleotide such that the control sequence directs expression of the coding sequence.

Sequence identity: The relatedness between two amino acid sequences or between two nucleotide sequences is described by the parameter “sequence identity”. For purposes of the present invention, the sequence identity between two amino acid sequences is determined using the Needleman-Wunsch algorithm (Needleman and Wunsch, 1970, J. Mol. Biol. 48: 443-453) as implemented in the Needle program of the EMBOSS package (EMBOSS: The European Molecular Biology Open Software Suite, Rice et al., 2000, Trends Genet. 16: 276-277), preferably version 5.0.0 or later. The parameters used are gap open penalty of 10, gap extension penalty of 0.5, and the EBLOSUM62 (EMBOSS version of BLOSUM62) substitution matrix. The output of Needle labeled “longest identity” (obtained using the −nobrief option) is used as the percent identity and is calculated as follows:

(Identical Residues×100)/(Length of Alignment−Total Number of Gaps in Alignment)

For purposes of the present invention, the sequence identity between two deoxyribonucleotide sequences is determined using the Needleman-Wunsch algorithm (Needleman and Wunsch, 1970, supra) as implemented in the Needle program of the EMBOSS package (EMBOSS: The European Molecular Biology Open Software Suite, Rice et al., 2000, supra), preferably version 5.0.0 or later. The parameters used are gap open penalty of 10, gap extension penalty of 0.5, and the EDNAFULL (EMBOSS version of NCBI NUC4.4) substitution matrix. The output of Needle labeled “longest identity” (obtained using the −nobrief option) is used as the percent identity and is calculated as follows:

(Identical Deoxyribonucleotides×100)/(Length of Alignment−Total Number of Gaps in Alignment)

Subsequence: The term “subsequence” means a polynucleotide having one or more (e.g., several) nucleotides absent from the 5′ and/or 3′ end of a mature polypeptide coding sequence; wherein the subsequence encodes a fragment having berberine bridge enzyme activity. In one aspect, a subsequence contains at least 35 nucleotides (e.g., nucleotides 546 to 581 of SEQ ID NO: 13), at least 40 nucleotides (e.g., nucleotides 502 to 541 of SEQ ID NO: 3), or at least 45 nucleotides (e.g., nucleotides 428 to 472 of SEQ ID NO: 1).

Variant: The term “variant” means a polypeptide having berberine bridge enzyme activity comprising an alteration, i.e., a substitution, insertion, and/or deletion, at one or more (e.g., several) positions. A substitution means replacement of the amino acid occupying a position with a different amino acid; a deletion means removal of the amino acid occupying a position; and an insertion means adding one or more (e.g., several) amino acids, e.g., 1-5 amino acids, adjacent to the amino acid occupying a position.

Very high stringency conditions: The term “very high stringency conditions” means for probes of at least 100 nucleotides in length, prehybridization and hybridization at 42° C. in 5×SSPE, 0.3% SDS, 200 micrograms/ml sheared and denatured salmon sperm DNA, and 50% formamide, following standard Southern blotting procedures for 12 to 24 hours. The carrier material is finally washed three times each for 15 minutes using 2×SSC, 0.2% SDS at 70° C.

Very low stringency conditions: The term “very low stringency conditions” means for probes of at least 100 nucleotides in length, prehybridization and hybridization at 42° C. in 5×SSPE, 0.3% SDS, 200 micrograms/ml sheared and denatured salmon sperm DNA, and 25% formamide, following standard Southern blotting procedures for 12 to 24 hours. The carrier material is finally washed three times each for 15 minutes using 2×SSC, 0.2% SDS at 45° C.

Cellulolytic activity: The term “cellulolytic activity” means a biological activity that hydrolyzes a cellulosic material. The two basic approaches for measuring cellulolytic activity include: (1) measuring the total cellulolytic activity, and (2) measuring the individual cellulolytic activities (endoglucanases, cellobiohydrolases, and beta-glucosidases) as reviewed in Zhang et al., Outlook for cellulase improvement: Screening and selection strategies, 2006, Biotechnology Advances 24: 452-481. Total cellulolytic activity is usually measured using insoluble substrates, including Whatman No. 1 filter paper, microcrystalline cellulose, bacterial cellulose, algal cellulose, cotton, pretreated lignocellulose, etc. The most common total cellulolytic activity assay is the filter paper assay using Whatman No. 1 filter paper as the substrate. The assay was established by the International Union of Pure and Applied Chemistry (IUPAC) (Ghose, 1987, Measurement of cellulase activities, Pure Appl. Chem. 59: 257-68).

For purposes of the present invention, unless otherwise stated, cellulolytic activity is determined by measuring the increase in hydrolysis of a cellulosic material by cellulolytic enzyme(s) under the following conditions: 1-20 mg of cellulolytic protein/g of cellulose in PCS for 3-7 days at 50-65° C. compared to a control hydrolysis without addition of cellulolytic protein. Typical conditions are 1 ml reactions, washed or unwashed PCS, 5% insoluble solids, 50 mM sodium acetate pH 5, 1 mM MnSO₄, 50-65° C., 72 hours, sugar analysis by AMINEX® HPX-87H column (Bio-Rad Laboratories, Inc., Hercules, Calif., USA).

Endoglucanase: The term “endoglucanase” means an endo-1,4-(1,3;1,4)-beta-D-glucan 4-glucanohydrolase (E.C. 3.2.1.4), which catalyses endohydrolysis of 1,4-beta-D-glycosidic linkages in cellulose, cellulose derivatives (such as carboxymethyl cellulose and hydroxyethyl cellulose), lichenin, beta-1,4 bonds in mixed beta-1,3 glucans such as cereal beta-D-glucans or xyloglucans, and other plant material containing cellulosic components. Endoglucanase activity can be determined by measuring reduction in substrate viscosity or increase in reducing ends determined by a reducing sugar assay (Zhang et al., 2006, Biotechnology Advances 24: 452-481). For purposes of the present invention, endoglucanase activity is determined using carboxymethyl cellulose (CMC) as substrate according to the procedure of Ghose, 1987, Pure and Appl. Chem. 59: 257-268, at pH 5, 40° C.

Cellobiohydrolase: The term “cellobiohydrolase” means a 1,4-beta-D-glucan cellobiohydrolase (E.C. 3.2.1.91), which catalyzes the hydrolysis of 1,4-beta-D-glucosidic linkages in cellulose, cellooligosaccharides, or any beta-1,4-linked glucose containing polymer, releasing cellobiose from the reducing or non-reducing ends of the chain (Teeri, 1997, Crystalline cellulose degradation: New insight into the function of cellobiohydrolases, Trends in Biotechnology 15: 160-167; Teeri et al., 1998, Trichoderma reesei cellobiohydrolases: why so efficient on crystalline cellulose?, Biochem. Soc. Trans. 26: 173-178). For purposes of the present invention, cellobiohydrolase activity is determined using a fluorescent disaccharide derivative 4-methylumbelliferyl-β-D-lactoside according to the procedures described by van Tilbeurgh et al., 1982, FEBS Letters 149: 152-156 and van Tilbeurgh and Claeyssens, 1985, FEBS Letters 187: 283-288, at pH 5, 40° C.

Beta-glucosidase: The term “beta-glucosidase” means a beta-D-glucoside glucohydrolase (E.C. 3.2.1.21), which catalyzes the hydrolysis of terminal non-reducing beta-D-glucose residues with the release of beta-D-glucose. For purposes of the present invention, beta-glucosidase activity is determined according to the basic procedure described by Venturi et al., 2002, Extracellular beta-D-glucosidase from Chaetomium thermophilum var. coprophilum: production, purification and some biochemical properties, J. Basic Microbiol. 42: 55-66. One unit of beta-glucosidase is defined as 1.0 μmole of p-nitrophenol produced per minute at 40° C., pH 5 from 1 mM p-nitrophenyl-beta-D-glucopyranoside as substrate in 100 mM sodium citrate containing 0.01% TWEEN® 20.

Cellulolytic enhancing activity: The term “cellulolytic enhancing activity” means a biological activity catalyzed by a polypeptide such as a GH61 or a BBEL polypeptide that enhances the hydrolysis of cellulosic material by enzymes having cellulolytic activity. For purposes of the present invention, cellulolytic enhancing activity is determined by measuring the increase in reducing sugars or the increase of the total of cellobiose and glucose from the hydrolysis of a cellulosic material by cellulolytic enzyme under the following conditions 5 mg of total protein/g of cellulose in PCS, wherein total protein is comprised of 50-99.5% w/w cellulolytic protein and 0.5-50% w/w protein of a GH61 polypeptide having cellulolytic enhancing activity and from 0-50 μg Berberine Bridge Enzyme Like (BBEL) for 1-7 day at 50-65° C. compared to a control hydrolysis with equal total protein loading without cellulolytic enhancing activity (1-50 mg of cellulolytic protein/g of cellulose in PCS). In a preferred aspect, a mixture of CELLUCLAST® 1.5 L (Novozymes A/S, Bagsværd, Denmark) in the presence of 3% of total protein weight Aspergillus oryzae beta-glucosidase (recombinantly produced in Aspergillus oryzae according to WO 02/095014) or 3% of total protein weight Aspergillus fumigatus beta-glucosidase (recombinantly produced in Aspergillus oryzae as described in WO 2002/095014) of cellulase protein loading is used as the source of the cellulolytic activity.

The polypeptides, such as a GH61 or a BBEL polypeptide having cellulolytic enhancing activity enhance the hydrolysis of a cellulosic material catalyzed by enzyme having cellulolytic activity by reducing the amount of cellulolytic enzyme required to reach the same degree of hydrolysis preferably at least 1.01-fold, more preferably at least 1.05-fold, more preferably at least 1.10-fold, more preferably at least 1.25-fold, more preferably at least 1.5-fold, more preferably at least 2-fold, more preferably at least 3-fold, more preferably at least 4-fold, more preferably at least 5-fold, even more preferably at least 10-fold, and most preferably at least 20-fold.

Family 61 glycoside hydrolase: The term “Family 61 glycoside hydrolase” or “Family GH61” or “GH61” means a polypeptide falling into the glycoside hydrolase Family 61 according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696.

Xylan degrading activity: The terms “xylan degrading activity” or “xylanolytic activity” mean a biological activity that hydrolyzes xylan-containing material. The two basic approaches for measuring xylanolytic activity include: (1) measuring the total xylanolytic activity, and (2) measuring the individual xylanolytic activities (endoxylanases, beta-xylosidases, arabinofuranosidases, alpha-glucuronidases, acetylxylan esterases, feruloyl esterases, and alpha-glucuronyl esterases). Assays of xylanolytic enzymes has been summarized in several publications including Biely and Puchard, Recent progress in the assays of xylanolytic enzymes, 2006, Journal of the Science of Food and Agriculture 86(11): 1636-1647; Spanikova and Biely, 2006, Glucuronoyl esterase—Novel carbohydrate esterase produced by Schizophyllum commune, FEBS Letters 580(19): 4597-4601; Herrmann, Vrsanska, Jurickova, Hirsch, Biely, and Kubicek, 1997, The beta-D-xylosidase of Trichoderma reesei is a multifunctional beta-D-xylan xylohydrolase, Biochemical Journal 321: 375-381.

Total xylan degrading activity can be measured by determining the reducing sugars formed from various types of xylan, including, for example, oat spelt, beechwood, and larchwood xylans, or by photometric determination of dyed xylan fragments released from various covalently dyed xylans. The most common total xylanolytic activity assay is based on production of reducing sugars from polymeric 4-O-methyl glucuronoxylan as described in Bailey, Biely, Poutanen, 1992, Interlaboratory testing of methods for assay of xylanase activity, Journal of Biotechnology 23(3): 257-270.

For purposes of the present invention, xylan degrading activity is determined by measuring the increase in hydrolysis of birchwood xylan (Sigma Chemical Co., Inc., St. Louis, Mo., USA) by xylan-degrading enzyme(s) under the following typical conditions: 1 ml reactions, 5 mg/ml substrate (total solids), 5 mg of xylanolytic protein/g of substrate, 50 mM sodium acetate pH 5, 50° C., 24 hours, sugar analysis using p-hydroxybenzoic acid hydrazide (PHBAH) assay as described by Lever, 1972, A new reaction for colorimetric determination of carbohydrates, Anal. Biochem 47: 273-279.

Xylanase: The term “xylanase” means a 1,4-beta-D-xylan-xylohydrolase (E.C. 3.2.1.8) that catalyzes the endo-hydrolysis of 1,4-beta-D-xylosidic linkages in xylans. For purposes of the present invention, xylanase activity is determined using birchwood xylan as substrate. One unit of xylanase is defined as 1.0 μmole of reducing sugar (measured in glucose equivalents as described by Lever, 1972, A new reaction for colorimetric determination of carbohydrates, Anal. Biochem 47: 273-279) produced per minute during the initial period of hydrolysis at 50° C., pH 5 from 2 g of birchwood xylan per liter as substrate in 50 mM sodium acetate containing 0.01% TWEEN® 20.

Beta-xylosidase: The term “beta-xylosidase” means a beta-D-xyloside xylohydrolase (E.C. 3.2.1.37) that catalyzes the exo-hydrolysis of short beta (1→4)-xylooligosaccharides, to remove successive D-xylose residues from the non-reducing termini. For purposes of the present invention, one unit of beta-xylosidase is defined as 1.0 μmole of p-nitrophenol produced per minute at 40° C., pH 5 from 1 mM p-nitrophenyl-beta-D-xyloside as substrate in 100 mM sodium citrate containing 0.01% TWEEN® 20.

Acetylxylan esterase: The term “acetylxylan esterase” means a carboxylesterase (EC 3.1.1.72) that catalyses the hydrolysis of acetyl groups from polymeric xylan, acetylated xylose, acetylated glucose, alpha-napthyl acetate, and p-nitrophenyl acetate. For purposes of the present invention, acetylxylan esterase activity is determined using 0.5 mM p-nitrophenylacetate as substrate in 50 mM sodium acetate pH 5.0 containing 0.01% TWEEN™ 20. One unit of acetylxylan esterase is defined as the amount of enzyme capable of releasing 1 μmole of p-nitrophenolate anion per minute at pH 5, 25° C.

Feruloyl esterase: The term “feruloyl esterase” means a 4-hydroxy-3-methoxycinnamoyl-sugar hydrolase (EC 3.1.1.73) that catalyzes the hydrolysis of the 4-hydroxy-3-methoxycinnamoyl (feruloyl) group from an esterified sugar, which is usually arabinose in “natural” substrates, to produce ferulate (4-hydroxy-3-methoxycinnamate). Feruloyl esterase is also known as ferulic acid esterase, hydroxycinnamoyl esterase, FAE-III, cinnamoyl ester hydrolase, FAEA, cinnAE, FAE-I, or FAE-II. For purposes of the present invention, feruloyl esterase activity is determined using 0.5 mM p-nitrophenylferulate as substrate in 50 mM sodium acetate pH 5.0. One unit of feruloyl esterase equals the amount of enzyme capable of releasing 1 μmole of p-nitrophenolate anion per minute at pH 5, 25° C.

Alpha-glucuronidase: The term “alpha-glucuronidase” means an alpha-D-glucosiduronate glucuronohydrolase (EC 3.2.1.139) that catalyzes the hydrolysis of an alpha-D-glucuronoside to D-glucuronate and an alcohol. For purposes of the present invention, alpha-glucuronidase activity is determined according to de Vries, 1998, J. Bacteriol. 180: 243-249. One unit of alpha-glucuronidase equals the amount of enzyme capable of releasing 1 μmole of glucuronic or 4-O-methylglucuronic acid per minute at pH 5, 40° C.

Alpha-L-arabinofuranosidase: The term “alpha-L-arabinofuranosidase” means an alpha-L-arabinofuranoside arabinofuranohydrolase (EC 3.2.1.55) that catalyzes the hydrolysis of terminal non-reducing alpha-L-arabinofuranoside residues in alpha-L-arabinosides. The enzyme acts on alpha-L-arabinofuranosides, alpha-L-arabinans containing (1,3)- and/or (1,5)-linkages, arabinoxylans, and arabinogalactans. Alpha-L-arabinofuranosidase is also known as arabinosidase, alpha-arabinosidase, alpha-L-arabinosidase, alpha-arabinofuranosidase, polysaccharide alpha-L-arabinofuranosidase, alpha-L-arabinofuranoside hydrolase, L-arabinosidase, or alpha-L-arabinanase. For purposes of the present invention, alpha-L-arabinofuranosidase activity is determined using 5 mg of medium viscosity wheat arabinoxylan (Megazyme International Ireland, Ltd., Bray, Co. Wicklow, Ireland) per ml of 100 mM sodium acetate pH 5 in a total volume of 200 μl for 30 minutes at 40° C. followed by arabinose analysis by AMINEX® HPX-87H column chromatography (Bio-Rad Laboratories, Inc., Hercules, Calif., USA).

Cellulosic material: The cellulosic material can be any material containing cellulose. The predominant polysaccharide in the primary cell wall of biomass is cellulose, the second most abundant is hemicellulose, and the third is pectin. The secondary cell wall, produced after the cell has stopped growing, also contains polysaccharides and is strengthened by polymeric lignin covalently cross-linked to hemicellulose. Cellulose is a homopolymer of anhydrocellobiose and thus a linear beta-(1-4)-D-glucan, while hemicelluloses include a variety of compounds, such as xylans, xyloglucans, arabinoxylans, and mannans in complex branched structures with a spectrum of substituents. Although generally polymorphous, cellulose is found in plant tissue primarily as an insoluble crystalline matrix of parallel glucan chains. Hemicelluloses usually hydrogen bond to cellulose, as well as to other hemicelluloses, which help stabilize the cell wall matrix.

Cellulose is generally found, for example, in the stems, leaves, hulls, husks, and cobs of plants or leaves, branches, and wood of trees. The cellulosic material can be, but is not limited to, herbaceous material, agricultural residue, forestry residue, municipal solid waste, waste paper, and pulp and paper mill residue (see, for example, Wiselogel et al., 1995, in Handbook on Bioethanol (Charles E. Wyman, editor), pp. 105-118, Taylor & Francis, Washington D.C.; Wyman, 1994, Bioresource Technology 50: 3-16; Lynd, 1990, Applied Biochemistry and Biotechnology 24/25: 695-719; Mosier et al, 1999, Recent Progress in Bioconversion of Lignocellulosics, in Advances in Biochemical Engineering/Biotechnology, T. Scheper, managing editor, Volume 65, pp. 23-40, Springer-Verlag, New York). It is understood herein that the cellulosic material may be in the form of lignocellulose, a plant cell wall material containing lignin, cellulose, and hemicellulose in a mixed matrix. In a preferred aspect, the cellulosic material is lignocellulose.

In one aspect, the cellulosic material is herbaceous material. In another aspect, the cellulosic material is agricultural residue. In another aspect, the cellulosic material is forestry residue. In another aspect, the cellulosic material is municipal solid waste. In another aspect, the cellulosic material is waste paper. In another aspect, the cellulosic material is pulp and paper mill residue.

In another aspect, the cellulosic material is corn stover. In another aspect, the cellulosic material is corn fiber. In another aspect, the cellulosic material is corn cob. In another aspect, the cellulosic material is orange peel. In another aspect, the cellulosic material is rice straw. In another aspect, the cellulosic material is wheat straw. In another aspect, the cellulosic material is switch grass. In another aspect, the cellulosic material is miscanthus. In another aspect, the cellulosic material is bagasse.

In another aspect, the cellulosic material is microcrystalline cellulose. In another aspect, the cellulosic material is bacterial cellulose. In another aspect, the cellulosic material is algal cellulose. In another aspect, the cellulosic material is cotton linter. In another aspect, the cellulosic material is amorphous phosphoric-acid treated cellulose. In another aspect, the cellulosic material is filter paper.

The cellulosic material may be used as is or may be subjected to pretreatment, using conventional methods known in the art, as described herein. In a preferred aspect, the cellulosic material is pretreated.

Pretreated corn stover: The term “PCS” or “Pretreated Corn Stover” means a cellulosic material derived from corn stover optionally by treatment with heat and dilute sulfuric acid.

Xylan-containing material: The term “xylan-containing material” means any material comprising a plant cell wall polysaccharide containing a backbone of beta-(1-4)-linked xylose residues. Xylans of terrestrial plants are heteropolymers possessing a beta-(1-4)-D-xylopyranose backbone, which is branched by short carbohydrate chains. They comprise D-glucuronic acid or its 4-O-methyl ether, L-arabinose, and/or various oligosaccharides, composed of D-xylose, L-arabinose, D- or L-galactose, and D-glucose. Xylan-type polysaccharides can be divided into homoxylans and heteroxylans, which include glucuronoxylans, (arabino)glucuronoxylans, (glucurono)arabinoxylans, arabinoxylans, and complex heteroxylans. See, for example, Ebringerova et al., 2005, Adv. Polym. Sci. 186: 1-67.

In the methods of the present invention, any material containing xylan may be used. In a preferred aspect, the xylan-containing material is lignocellulose.

### DETAILED DESCRIPTION OF THE INVENTION

Polypeptides Having Berberine Bridge Like Enzyme activity

In an embodiment, the present invention relates to isolated polypeptides having a sequence identity to the mature polypeptide of SEQ ID NO: 2 of at least 95%, e.g. at least 96%, at least 97%, at least 98%, at least 99%, or 100%, which have berberine bridge enzyme activity or

a sequence identity to the mature polypeptide of SEQ ID NO: 4 of at least 90%, e.g., at least 91%, at least 92%, at least 93%, at least 94%, at least 95%, at least 96%, at least 97%, at least 98%, at least 99%, or 100%, which have Berberine Bridge Enzyme Like activity or

a sequence identity to the mature polypeptide of SEQ ID NO: 6 of at least 80%, e.g., at least 85%, at least 90%, at least 91%, at least 92%, at least 93%, at least 94%, at least 95%, at least 96%, at least 97%, at least 98%, at least 99%, or 100%, which have Berberine Bridge Enzyme Like activity or

a sequence identity to the mature polypeptide of SEQ ID NO: 8 of at least 80%, e.g., at least 85%, at least 90%, at least 91%, at least 92%, at least 93%, at least 94%, at least 95%, at least 96%, at least 97%, at least 98%, at least 99%, or 100%, which have Berberine Bridge Enzyme Like activity or

a sequence identity to the mature polypeptide of SEQ ID NO: 10 of at least 80%, e.g., at least 85%, at least 90%, at least 91%, at least 92%, at least 93%, at least 94%, at least 95%, at least 96%, at least 97%, at least 98%, at least 99%, or 100%, which have Berberine Bridge Enzyme Like activity or

a sequence identity to the mature polypeptide of SEQ ID NO: 12 of at least 75%, e.g., at least 80%, at least 85%, at least 90%, at least 91%, at least 92%, at least 93%, at least 94%, at least 95%, at least 96%, at least 97%, at least 98%, at least 99%, or 100%, which have Berberine Bridge Enzyme Like activity or

a sequence identity to the mature polypeptide of SEQ ID NO: 14 of at least 75%, e.g., at least 80%, at least 85%, at least 90%, at least 91%, at least 92%, at least 93%, at least 94%, at least 95%, at least 96%, at least 97%, at least 98%, at least 99%, or 100%, which have Berberine Bridge Enzyme Like activity or

a sequence identity to the mature polypeptide of SEQ ID NO: 16 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%, which have Berberine Bridge Enzyme Like activity. In one aspect, the polypeptides differ by no more than 10 amino acids, e.g., 1, 2, 3, 4, 5, 6, 7, 8, or 9, from the mature polypeptide of SEQ ID NO: 2 or no more than 10 amino acids, e.g., 1, 2, 3, 4, 5, 6, 7, 8, or 9, from the mature polypeptide of SEQ ID NO: 4, or no more than 10 amino acids, e.g., 1, 2, 3, 4, 5, 6, 7, 8, or 9, from the mature polypeptide of SEQ ID NO: 6, or no more than 10 amino acids, e.g., 1, 2, 3, 4, 5, 6, 7, 8, or 9, from the mature polypeptide of SEQ ID NO: 8, or no more than 10 amino acids, e.g., 1, 2, 3, 4, 5, 6, 7, 8, or 9, from the mature polypeptide of SEQ ID NO: 10, or no more than 10 amino acids, e.g., 1, 2, 3, 4, 5, 6, 7, 8, or 9, from the mature polypeptide of SEQ ID NO: 12, or no more than 10 amino acids, e.g., 1, 2, 3, 4, 5, 6, 7, 8, or 9, from the mature polypeptide of SEQ ID NO: 14 or no more than 10 amino acids, e.g., 1, 2, 3, 4, 5, 6, 7, 8, or 9, from the mature polypeptide of SEQ ID NO: 16.

A polypeptide of the present invention preferably comprises or consists of the amino acid sequence of SEQ ID NO: 2 or an allelic variant thereof; or is a fragment thereof having Berberine Bridge Enzyme Like activity. In another aspect, the polypeptide comprises or consists of the mature polypeptide of SEQ ID NO: 2.

In another embodiment, the present invention relates to an isolated polypeptide having Berberine Bridge Enzyme Like activity encoded by a polynucleotide that hybridizes under medium stringency conditions, medium-high stringency conditions, high stringency conditions, or very high stringency conditions with (i) the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, (ii) the cDNA sequence thereof, or (iii) the full-length complement of (i) or (ii) (Sambrook et al., 1989, Molecular Cloning, A Laboratory Manual, 2 d edition, Cold Spring Harbor, N.Y.).

The polynucleotide of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 or a subsequence thereof, as well as the polypeptide of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16 or a fragment thereof, may be used to design nucleic acid probes to identify and clone DNA encoding polypeptides having Berberine Bridge Enzyme Like activity from strains of different genera or species according to methods well known in the art. In particular, such probes can be used for hybridization with the genomic DNA or cDNA of a cell of interest, following standard Southern blotting procedures, in order to identify and isolate the corresponding gene therein. Such probes can be considerably shorter than the entire sequence, but should be at least 15, e.g., at least 25, at least 35, or at least 70 nucleotides in length. Preferably, the nucleic acid probe is at least 100 nucleotides in length, e.g., at least 200 nucleotides, at least 300 nucleotides, at least 400 nucleotides, at least 500 nucleotides, at least 600 nucleotides, at least 700 nucleotides, at least 800 nucleotides, or at least 900 nucleotides in length. Both DNA and RNA probes can be used. The probes are typically labeled for detecting the corresponding gene (for example, with ³²P, ³H, ³⁵S, biotin, or avidin). Such probes are encompassed by the present invention.

A genomic DNA or cDNA library prepared from such other strains may be screened for DNA that hybridizes with the probes described above and encodes a polypeptide having Berberine Bridge Enzyme Like activity. Genomic or other DNA from such other strains may be separated by agarose or polyacrylamide gel electrophoresis, or other separation techniques. DNA from the libraries or the separated DNA may be transferred to and immobilized on nitrocellulose or other suitable carrier material. In order to identify a clone or DNA that hybridizes with SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 or a subsequence thereof, the carrier material is used in a Southern blot.

For purposes of the present invention, hybridization indicates that the polynucleotide hybridizes to a labeled nucleic acid probe corresponding to (i) SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15; (ii) the mature polypeptide coding sequence of; (iii) the cDNA sequence thereof; (iv) the full-length complement thereof; or (v) a subsequence thereof; under very low to very high stringency conditions. Molecules to which the nucleic acid probe hybridizes under these conditions can be detected using, for example, X-ray film or any other detection means known in the art.

In one aspect, the nucleic acid probe is encoding at least a part of a catalytic domain of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16. In another aspect, the nucleic acid probe is a polynucleotide that encodes the polypeptide of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16; the mature polypeptide thereof; or a fragment thereof. In another aspect, the nucleic acid probe is SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 or the cDNA sequence thereof.

In another embodiment, the present invention relates to an isolated polypeptide having Berberine Bridge Enzyme Like activity encoded by a polynucleotide having a sequence identity to the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention relates to variants of the mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16 comprising a substitution, deletion, and/or insertion at one or more (e.g., several) positions. In an embodiment, the number of amino acid substitutions, deletions and/or insertions introduced into the mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16 is not more than 10, e.g., 1, 2, 3, 4, 5, 6, 7, 8 or 9. The amino acid changes may be of a minor nature, that is conservative amino acid substitutions or insertions that do not significantly affect the folding and/or activity of the protein; small deletions, typically of 1-30 amino acids; small amino- or carboxyl-terminal extensions, such as an amino-terminal methionine residue; a small linker peptide of up to 20-25 residues; or a small extension that facilitates purification by changing net charge or another function, such as a poly-histidine tract, an antigenic epitope or a binding domain.

Examples of conservative substitutions are within the groups of basic amino acids (arginine, lysine and histidine), acidic amino acids (glutamic acid and aspartic acid), polar amino acids (glutamine and asparagine), hydrophobic amino acids (leucine, isoleucine and valine), aromatic amino acids (phenylalanine, tryptophan and tyrosine), and small amino acids (glycine, alanine, serine, threonine and methionine). Amino acid substitutions that do not generally alter specific activity are known in the art and are described, for example, by H. Neurath and R. L. Hill, 1979, In, The Proteins, Academic Press, New York. Common substitutions are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly.

Alternatively, the amino acid changes are of such a nature that the physico-chemical properties of the polypeptides are altered. For example, amino acid changes may improve the thermal stability of the polypeptide, alter the substrate specificity, change the pH optimum, and the like.

Essential amino acids in a polypeptide can be identified according to procedures known in the art, such as site-directed mutagenesis or alanine-scanning mutagenesis (Cunningham and Wells, 1989, Science 244: 1081-1085). In the latter technique, single alanine mutations are introduced at every residue in the molecule, and the resultant mutant molecules are tested for Berberine Bridge Enzyme Like activity to identify amino acid residues that are critical to the activity of the molecule. See also, Hilton et al., 1996, J. Biol. Chem. 271: 4699-4708. The active site of the enzyme or other biological interaction can also be determined by physical analysis of structure, as determined by such techniques as nuclear magnetic resonance, crystallography, electron diffraction, or photoaffinity labeling, in conjunction with mutation of putative contact site amino acids. See, for example, de Vos et al., 1992, Science 255: 306-312; Smith et al., 1992, J. Mol. Biol. 224: 899-904; Wlodaver et al., 1992, FEBS Lett. 309: 59-64. The identity of essential amino acids can also be inferred from an alignment with a related polypeptide as provided in FIG. 3 herein. It is understood in the art, that amendments of amino acids in regions with limited conservation (i.e. high variability) are expected to entail limited functional changes in the peptide, whereas changes in regions showing a high degree of conservation between peptides with a similar function are expected to bring significant changes in the function of he peptide.

Single or multiple amino acid substitutions, deletions, and/or insertions can be made and tested using known methods of mutagenesis, recombination, and/or shuffling, followed by a relevant screening procedure, such as those disclosed by Reidhaar-Olson and Sauer, 1988, Science 241: 53-57; Bowie and Sauer, 1989, Proc. Natl. Acad. Sci. USA 86: 2152-2156; WO 95/17413; or WO 95/22625. Other methods that can be used include error-prone PCR, phage display (e.g., Lowman et al., 1991, Biochemistry 30: 10832-10837; U.S. Pat. No. 5,223,409; WO 92/06204), and region-directed mutagenesis (Derbyshire et al, 1986, Gene 46: 145; Ner et al., 1988, DNA 7: 127).

Mutagenesis/shuffling methods can be combined with high-throughput, automated screening methods to detect activity of cloned, mutagenized polypeptides expressed by host cells (Ness et al., 1999, Nature Biotechnology 17: 893-896). Mutagenized DNA molecules that encode active polypeptides can be recovered from the host cells and rapidly sequenced using standard methods in the art. These methods allow the rapid determination of the importance of individual amino acid residues in a polypeptide.

The polypeptide may be a hybrid polypeptide in which a region of one polypeptide is fused at the N-terminus or the C-terminus of a region of another polypeptide.

The polypeptide may be a fusion polypeptide or cleavable fusion polypeptide in which another polypeptide is fused at the N-terminus or the C-terminus of the polypeptide of the present invention. A fusion polypeptide is produced by fusing a polynucleotide encoding another polypeptide to a polynucleotide of the present invention. Techniques for producing fusion polypeptides are known in the art, and include ligating the coding sequences encoding the polypeptides so that they are in frame and that expression of the fusion polypeptide is under control of the same promoter(s) and terminator. Fusion polypeptides may also be constructed using intein technology in which fusion polypeptides are created post-translationally (Cooper et al., 1993, EMBO J. 12: 2575-2583; Dawson et al, 1994, Science 266: 776-779).

A fusion polypeptide can further comprise a cleavage site between the two polypeptides. Upon secretion of the fusion protein, the site is cleaved releasing the two polypeptides. Examples of cleavage sites include, but are not limited to, the sites disclosed in Martin et al., 2003, J. Ind. Microbiol. Biotechnol. 3: 568-576; Svetina et al., 2000, J. Biotechnol. 76: 245-251; Rasmussen-Wilson et al., 1997, Appl. Environ. Microbiol. 63: 3488-3493; Ward et al., 1995, Biotechnology 13: 498-503; and Contreras et al., 1991, Biotechnology 9: 378-381; Eaton et al., 1986, Biochemistry 25: 505-512; Collins-Racie et al., 1995, Biotechnology 13: 982-987; Carter et al., 1989, Proteins: Structure, Function, and Genetics 6: 240-248; and Stevens, 2003, Drug Discovery World 4: 35-48.

### Sources of Polypeptides Having Berberine Bridge Enzyme Like (BBEL) Activity

A polypeptide having Berberine Bridge Enzyme Like activity of the present invention may be obtained from microorganisms of any genus. For purposes of the present invention, the term “obtained from” as used herein in connection with a given source shall mean that the polypeptide encoded by a polynucleotide is produced by the source or by a strain in which the polynucleotide from the source has been inserted. In one aspect, the polypeptide obtained from a given source is secreted extracellularly.

The polypeptide may be a fungal polypeptide. For example, the polypeptide may be a yeast polypeptide such as a Candida, Kluyveromyces, Pichia, Saccharomyces, Schizosaccharomyces, or Yarrowia polypeptide; or a filamentous fungal polypeptide such as an Acremonium, Agaricus, Alternaria, Aspergillus, Aureobasidium, Botryospaeria, Ceriporiopsis, Chaetomidium, Chrysosporium, Claviceps, Cochliobolus, Coprinopsis, Coptotermes, Corynascus, Cryphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide.

In another aspect, the polypeptide is an Acremonium cellulolyticus, Aspergillus aculeatus, Aspergillus awamori, Aspergillus foetidus, Aspergillus fumigatus, Aspergillus japonicus, Aspergillus nidulans, Aspergillus niger, Aspergillus oryzae, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia setosa, Thielavia spededonium, Thielavia subthermophila, Thielavia terrestris, Trichoderma harzianum, Trichoderma koningii, Trichoderma longibrachiatum, Trichoderma reesei, or Trichoderma viride polypeptide.

In another aspect, the polypeptide is an Aspergillus or Penicillium polypeptide, e.g., a polypeptide obtained from Aspergillus aculeatus or Penicillium pinophilum.

It will be understood that for the aforementioned species, the invention encompasses both the perfect and imperfect states, and other taxonomic equivalents, e.g., anamorphs, regardless of the species name by which they are known. Those skilled in the art will readily recognize the identity of appropriate equivalents.

Strains of these species are readily accessible to the public in a number of culture collections, such as the American Type Culture Collection (ATCC), Deutsche Sammlung von Mikroorganismen and Zellkulturen GmbH (DSMZ), Centraalbureau Voor Schimmelcultures (CBS), and Agricultural Research Service Patent Culture Collection, Northern Regional Research Center (NRRL).

The polypeptide may be identified and obtained from other sources including microorganisms and plants isolated from nature (e.g., soil, composts, water, etc.) or DNA samples obtained directly from natural materials (e.g., soil, composts, water, plants etc.) using the above-mentioned probes. Techniques for isolating microorganisms and DNA directly from natural habitats are well known in the art. A polynucleotide encoding the polypeptide may then be obtained by similarly screening a genomic DNA or cDNA library of another microorganism or mixed DNA sample. Once a polynucleotide encoding a polypeptide has been detected with the probe(s), the polynucleotide can be isolated or cloned by utilizing techniques that are known to those of ordinary skill in the art (see, e.g., Sambrook et al., 1989, supra).

### Catalytic Domains

In one embodiment, the present invention also relates to catalytic domains having a sequence identity to amino acids 428 to 472 of SEQ ID NO: 2, of at least 60%, e.g., at least 65%, at least 70%, at least 75%, at least 80%, at least 85%, at least 90%, at least 95%, at least 96%, at least 97%, at least 98%, at least 99%, or 100% or to amino acids 502 to 541 of SEQ ID NO: 4 of at least 60%, e.g., at least 65%, at least 70%, at least 75%, at least 80%, at least 85%, at least 90%, at least 95%, at least 96%, at least 97%, at least 98%, at least 99%, or 100% or amino acids 463 to 503 of SEQ ID NO: 6 of at least 60%, e.g., at least 65%, at least 70%, at least 75%, at least 80%, at least 85%, at least 90%, at least 95%, at least 96%, at least 97%, at least 98%, at least 99%, or 100% or amino acids 431 to 473 of SEQ ID NO: 8 of at least 60%, e.g., at least 65%, at least 70%, at least 75%, at least 80%, at least 85%, at least 90%, at least 95%, at least 96%, at least 97%, at least 98%, at least 99%, or 100% or amino acids 572 to 615 of SEQ ID NO: 10 of at least 60%, e.g., at least 65%, at least 70%, at least 75%, at least 80%, at least 85%, at least 90%, at least 95%, at least 96%, at least 97%, at least 98%, at least 99%, or 100% or amino acids 517 to 561 of SEQ ID NO: 12 of at least 60%, e.g., at least 65%, at least 70%, at least 75%, at least 80%, at least 85%, at least 90%, at least 95%, at least 96%, at least 97%, at least 98%, at least 99%, or 100% or amino acids 546 to 581 of SEQ ID NO: 14 of at least 60%, e.g., at least 65%, at least 70%, at least 75%, at least 80%, at least 85%, at least 90%, at least 95%, at least 96%, at least 97%, at least 98%, at least 99%, or 100% or 448 to 484 of SEQ ID NO: 16 of at least 60%, e.g., at least 65%, at least 70%, at least 75%, at least 80%, at least 85%, at least 90%, at least 95%, at least 96%, at least 97%, at least 98%, at least 99%, or 100%. In one aspect, the catalytic domains comprise amino acid sequences that differ by no more than 10 amino acids, e.g., 1, 2, 3, 4, 5, 6, 7, 8, or 9, from amino acids 428 to 472 of SEQ ID NO: 2, 502 to 541 of SEQ ID NO: 4, 463 to 503 of SEQ ID NO: 6, 431 to 473 of SEQ ID NO: 8, 572 to 615 of SEQ ID NO: 10, 517 to 561 of SEQ ID NO: 12, 546 to 581 of SEQ ID NO: 14 or 448 to 484 of SEQ ID NO: 16.

The catalytic domain preferably comprises or consists of amino acids 428 to 472 of SEQ ID NO: 2, 502 to 541 of SEQ ID NO: 4, 463 to 503 of SEQ ID NO: 6, 431 to 473 of SEQ ID NO: 8, 572 to 615 of SEQ ID NO: 10, 517 to 561 of SEQ ID NO: 12, 546 to 581 of SEQ ID NO: 14 or 448 to 484 of SEQ ID NO: 16 or an allelic variant thereof; or is a fragment thereof having Berberine Bridge Enzyme Like activity.

In another embodiment, the present invention also relates to catalytic domains encoded by polynucleotides that hybridize under medium stringency conditions, medium-high stringency conditions, high stringency conditions, or very high stringency conditions (as defined above) with (i) nucleotides 1370 to 1504 of SEQ ID NO: 1, 1615 to 1629 and/or 1680 to 1784 of SEQ ID NO: 3, 1498 to 1620 of SEQ ID NO: 5, 1575 to 1703 of SEQ ID NO: 7, 1834 to 1965 of SEQ ID NO: 9, 1649 to 1783 of SEQ ID NO: 11, 1697 to 1804 of SEQ ID NO: 13 or 1400 to 1401 and/or 1511 to 1619 of SEQ ID NO: 15, (ii) the cDNA sequence thereof, or (iii) the full-length complement of (i) or (ii) (Sambrook et al., 1989, supra).

In another embodiment, the present invention also relates to catalytic domains encoded by polynucleotides having a sequence identity to nucleotides 1370 to 1504 of SEQ ID NO: 1, 1615 to 1629 and/or 1680 to 1784 of SEQ ID NO: 3, 1498 to 1620 of SEQ ID NO: 5, 1575 to 1703 of SEQ ID NO: 7, 1834 to 1965 of SEQ ID NO: 9, 1649 to 1783 of SEQ ID NO: 11, 1697 to 1804 of SEQ ID NO: 13 or 1400 to 1401 and/or 1511 to 1619 of SEQ ID NO: 15 of at least 80%, at least 85%, at least 90%, at least 95%, at least 96%, at least 97%, at least 98%, at least 99%, or 100%.

The polynucleotide encoding the catalytic domain preferably comprises or consists of nucleotides, 1370 to 1504 of SEQ ID NO: 1, 1615 to 1629 and/or 1680 to 1784 of SEQ ID NO: 3, 1498 to 1620 of SEQ ID NO: 5, 1575 to 1703 of SEQ ID NO: 7, 1834 to 1965 of SEQ ID NO: 9, 1649 to 1783 of SEQ ID NO: 11, 1697 to 1804 of SEQ ID NO: 13 or 1400 to 1401 and/or 1511 to 1619 of SEQ ID NO: 15.

In another embodiment, the present invention also relates to catalytic domain variants of amino acids 428 to 472 of SEQ ID NO: 2, 502 to 541 of SEQ ID NO: 4, 463 to 503 of SEQ ID NO: 6, 431 to 473 of SEQ ID NO: 8, 572 to 615 of SEQ ID NO: 10, 517 to 561 of SEQ ID NO: 12, 546 to 581 of SEQ ID NO: 14 or 448 to 484 of SEQ ID NO: 16 comprising a substitution, deletion, and/or insertion at one or more (e.g., several) positions. In one aspect, the number of amino acid substitutions, deletions and/or insertions introduced into the sequence of amino acids 428 to 472 of SEQ ID NO: 2, 502 to 541 of SEQ ID NO: 4, 463 to 503 of SEQ ID NO: 6, 431 to 473 of SEQ ID NO: 8, 572 to 615 of SEQ ID NO: 10, 517 to 561 of SEQ ID NO: 12, 546 to 581 of SEQ ID NO: 14 or 448 to 484 of SEQ ID NO: 16 is 10, e.g., 1, 2, 3, 4, 5, 6, 8, or 9.

### Binding Domains

In one embodiment, the present invention also relates to FAD binding domains having a sequence identity to amino acids 48-186 of SEQ ID NO: 2 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100%, or a sequence identity to amino acids 122-256 of SEQ ID NO: 4 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100%, or a sequence identity to amino acids 80-213 of SEQ ID NO: 6 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100%, or a sequence identity to amino acids 54-188 of SEQ ID NO: 8 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100%, or a sequence identity to amino acids 179-319 of SEQ ID NO: 10 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100%, or a sequence identity to amino acids 128-265 of SEQ ID NO: 12 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100%, or a sequence identity to amino acids 119-261 of SEQ ID NO: 14 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100%, or a sequence identity to amino acids 63-199 of SEQ ID NO: 16 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100%. In one aspect, the FAD binding domains comprise amino acid sequences that differ by no more than 10 amino acids, e.g., 1, 2, 3, 4, 5, 6, 7, 8, or 9, from amino acids 48-186 of SEQ ID NO: 2, 122-256 of SEQ ID NO: 4, 80-213 of SEQ ID NO: 6, 54-188 of SEQ ID NO: 8, 179-319 of SEQ ID NO: 10, 128-265 of SEQ ID NO: 12, 119-261 of SEQ ID NO: 14 or 63-199 of SEQ ID NO: 16.

The FAD binding domain preferably comprises or consists of amino acids 48-186 of SEQ ID NO: 2, 122-256 of SEQ ID NO: 4, 80-213 of SEQ ID NO: 6, 54-188 of SEQ ID NO: 8, 179-319 of SEQ ID NO: 10, 128-265 of SEQ ID NO: 12, 119-261 of SEQ ID NO: 14 or 63-199 of SEQ ID NO: 16 or an allelic variant thereof; or is a fragment thereof having FAD binding activity.

In another embodiment, the present invention also relates to FAD binding domains encoded by polynucleotides that hybridize under medium stringency conditions, medium-high stringency conditions, high stringency conditions, or very high stringency conditions (as defined above) with (i) the nucleotides 142 to 259 and/or 348 to 646 of SEQ ID NO: 1, 424 to 509 and/or 561 to 879 of SEQ ID NO: 3, 238 to 279 and/or 338 to 697 of SEQ ID NO: 5, 160 to 201 and/or 266 to 482, 548 to 693 of SEQ ID NO: 7, 590 to 1012 of SEQ ID NO: 9, 433 to 524 and/or 574 to 895 of SEQ ID NO: 11, 355 to 470 and/or 532 to 844 of SEQ ID NO: 13, or 187 to 228 and/or 287 to 655 of SEQ ID NO: 15, (ii) the cDNA sequence thereof, or (iii) the full-length complement of (i) or (ii) (Sambrook et al., 1989, supra).

In another embodiment, the present invention also relates to FAD binding domains encoded by polynucleotides having a sequence identity to nucleotides 142 to 259 and/or 348 to 646 of SEQ ID NO: 1, 424 to 509 and/or 561 to 879 of SEQ ID NO: 3, 238 to 279 and/or 338 to 697 of SEQ ID NO: 5, 160 to 201 and/or 266 to 482, 548 to 693 of SEQ ID NO: 7, 90 to 1012 of SEQ ID NO: 9, 433 to 524 and/or 574 to 895 of SEQ ID NO: 11, 355 to 470 and/or 532 to 844 of SEQ ID NO: 13, or 187 to 228 and/or 287 to 655 of SEQ ID NO: 15 of at least 60%, e.g., at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or 100%.

In another embodiment, the present invention also relates to FAD binding domain variants of amino acids 48-186 of SEQ ID NO: 2, 122-256 of SEQ ID NO: 4, 80-213 of SEQ ID NO: 6, 54-188 of SEQ ID NO: 8, 179-319 of SEQ ID NO: 10, 128-265 of SEQ ID NO: 12, 119-261 of SEQ ID NO: 14 or 63-199 of SEQ ID NO: 16 comprising a substitution, deletion, and/or insertion at one or more (e.g., several) positions. In one aspect, the number of amino acid substitutions, deletions and/or insertions introduced into the sequence of amino acids 48-186 of SEQ ID NO: 2, 122-256 of SEQ ID NO: 4, 80-213 of SEQ ID NO: 6, 54-188 of SEQ ID NO: 8, 179-319 of SEQ ID NO: 10, 128-265 of SEQ ID NO: 12, 119-261 of SEQ ID NO: 14 or 63-199 of SEQ ID NO: 16 is 10, e.g., 1, 2, 3, 4, 5, 6, 8, or 9.

A catalytic domain operably linked to the FAD binding domain may be from hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase. In a related aspect, the FAD binding domain may be from a BBEL peptide. The polynucleotide encoding the catalytic domain may be obtained from any prokaryotic, eukaryotic, or other source.

### Polynucleotides

The present invention also relates to isolated polynucleotides encoding a polypeptide, a catalytic domain, or FAD binding domain of the present invention, as described herein.

The techniques used to isolate or clone a polynucleotide are known in the art and include isolation from genomic DNA or cDNA, or a combination thereof. The cloning of the polynucleotides from genomic DNA can be effected, e.g., by using the well known polymerase chain reaction (PCR) or antibody screening of expression libraries to detect cloned DNA fragments with shared structural features. See, e.g., Innis et al., 1990, PCR: A Guide to Methods and Application, Academic Press, New York. Other nucleic acid amplification procedures such as ligase chain reaction (LCR), ligation activated transcription (LAT) and polynucleotide-based amplification (NASBA) may be used. The polynucleotides may be cloned from a strain of Aspergillus or Penicillium, or a related organism and thus, for example, may be an allelic or species variant of the polypeptide encoding region of the polynucleotide.

Modification of a polynucleotide encoding a polypeptide of the present invention may be necessary for synthesizing polypeptides substantially similar to the polypeptide. The term “substantially similar” to the polypeptide refers to non-naturally occurring forms of the polypeptide. These polypeptides may differ in some engineered way from the polypeptide isolated from its native source, e.g., variants that differ in specific activity, thermostability, pH optimum, or the like. The variants may be constructed on the basis of the polynucleotide presented as the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15 or the cDNA sequence thereof, e.g., a subsequence thereof, and/or by introduction of nucleotide substitutions that do not result in a change in the amino acid sequence of the polypeptide, but which correspond to the codon usage of the host organism intended for production of the enzyme, or by introduction of nucleotide substitutions that may give rise to a different amino acid sequence. For a general description of nucleotide substitution, see, e.g., Ford et al., 1991, Protein Expression and Purification 2: 95-107.

### Nucleic Acid Constructs

The present invention also relates to nucleic acid constructs comprising a polynucleotide of the present invention operably linked to one or more control sequences that direct the expression of the coding sequence in a suitable host cell under conditions compatible with the control sequences.

A polynucleotide may be manipulated in a variety of ways to provide for expression of the polypeptide. Manipulation of the polynucleotide prior to its insertion into a vector may be desirable or necessary depending on the expression vector and intended host cell. The techniques for modifying polynucleotides utilizing recombinant DNA methods are well known in the art.

The control sequence may be a promoter, a polynucleotide that is recognized by a host cell for expression of a polynucleotide encoding a polypeptide of the present invention. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either homologous or heterologous to the host cell.

Examples of suitable promoters for directing transcription of the nucleic acid constructs of the present invention in a bacterial host cell are the promoters obtained from the Bacillus amyloliquefaciens alpha-amylase gene (amyQ), Bacillus licheniformis alpha-amylase gene (amyL), Bacillus licheniformis penicillinase gene (penP), Bacillus stearothermophilus maltogenic amylase gene (amyM), Bacillus subtilis levansucrase gene (sacB), Bacillus subtilis xyIA and xyIB genes, Bacillus thuringiensis cryIIIA gene (Agaisse and Lereclus, 1994, Molecular Microbiology 13: 97-107), E. coli lac operon, E. coli trc promoter (Egon et al., 1988, Gene 69: 301-315), Streptomyces coelicolor agarase gene (dagA), and prokaryotic beta-lactamase gene (Villa-Kamaroff et al., 1978, Proc. Natl. Acad. Sci. USA 75: 3727-3731), as well as the tac promoter (DeBoer et al., 1983, Proc. Natl. Acad. Sci. USA 80: 21-25). Further promoters are described in “Useful proteins from recombinant bacteria” in Gilbert et al., 1980, Scientific American 242: 74-94; and in Sambrook et al., 1989, supra. Examples of tandem promoters are disclosed in WO 99/43835.

Examples of suitable promoters for directing transcription of the nucleic acid constructs of the present invention in a filamentous fungal host cell are promoters obtained from the genes for Aspergillus nidulans acetamidase, Aspergillus niger neutral alpha-amylase, Aspergillus niger acid stable alpha-amylase, Aspergillus niger or Aspergillus awamori glucoamylase (glaA), Aspergillus oryzae TAKA amylase, Aspergillus oryzae alkaline protease, Aspergillus oryzae triose phosphate isomerase, Fusarium oxysporum trypsin-like protease (WO 96/00787), Fusarium venenatum amyloglucosidase (WO 00/56900), Fusarium venenatum Daria (WO 00/56900), Fusarium venenatum Quinn (WO 00/56900), Rhizomucor miehei lipase, Rhizomucor miehei aspartic proteinase, Trichoderma reesei beta-glucosidase, Trichoderma reesei cellobiohydrolase I, Trichoderma reesei cellobiohydrolase II, Trichoderma reesei endoglucanase I, Trichoderma reesei endoglucanase II, Trichoderma reesei endoglucanase III, Trichoderma reesei endoglucanase IV, Trichoderma reesei endoglucanase V, Trichoderma reesei xylanase I, Trichoderma reesei xylanase II, Trichoderma reesei beta-xylosidase, as well as the NA2-tpi promoter (a modified promoter from an Aspergillus neutral alpha-amylase gene in which the untranslated leader has been replaced by an untranslated leader from an Aspergillus triose phosphate isomerase gene; non-limiting examples include modified promoters from an Aspergillus niger neutral alpha-amylase gene in which the untranslated leader has been replaced by an untranslated leader from an Aspergillus nidulans or Aspergillus oryzae triose phosphate isomerase gene); and mutant, truncated, and hybrid promoters thereof.

In a yeast host, useful promoters are obtained from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al, 1992, Yeast 8: 423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used in the present invention.

Preferred terminators for bacterial host cells are obtained from the genes for Bacillus clausii alkaline protease (aprH), Bacillus licheniformis alpha-amylase (amyL), and Escherichia coli ribosomal RNA (rmB).

Preferred terminators for filamentous fungal host cells are obtained from the genes for Aspergillus nidulans anthranilate synthase, Aspergillus nigerglucoamylase, Aspergillus niger alpha-glucosidase, Aspergillus oryzae TAKA amylase, and Fusarium oxysporum trypsin-like protease.

Preferred terminators for yeast host cells are obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are described by Romanos et al., 1992, supra.

The control sequence may also be an mRNA stabilizer region downstream of a promoter and upstream of the coding sequence of a gene which increases expression of the gene.

Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465-3471).

The control sequence may also be a leader, a nontranslated region of an mRNA that is important for translation by the host cell. The leader is operably linked to the 5′-terminus of the polynucleotide encoding the polypeptide. Any leader that is functional in the host cell may be used.

Preferred leaders for filamentous fungal host cells are obtained from the genes for Aspergillus otyzae TAKA amylase and Aspergillus nidulans triose phosphate isomerase.

Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP).

The control sequence may also be a polyadenylation sequence, a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

Preferred polyadenylation sequences for filamentous fungal host cells are obtained from the genes for Aspergillus nidulans anthranilate synthase, Aspergillus niger glucoamylase, Aspergillus niger alpha-glucosidase Aspergillus oryzae TAKA amylase, and Fusarium oxysporum trypsin-like protease.

Useful polyadenylation sequences for yeast host cells are described by Guo and Sherman, 1995, Mol. Cellular Biol. 15: 5983-5990.

The control sequence may also be a signal peptide coding region that encodes a signal peptide linked to the N-terminus of a polypeptide and directs the polypeptide into the cell's secretory pathway. The 5′-end of the coding sequence of the polynucleotide may inherently contain a signal peptide coding sequence naturally linked in translation reading frame with the segment of the coding sequence that encodes the polypeptide. Alternatively, the 5′-end of the coding sequence may contain a signal peptide coding sequence that is foreign to the coding sequence. A foreign signal peptide coding sequence may be required where the coding sequence does not naturally contain a signal peptide coding sequence. Alternatively, a foreign signal peptide coding sequence may simply replace the natural signal peptide coding sequence in order to enhance secretion of the polypeptide. However, any signal peptide coding sequence that directs the expressed polypeptide into the secretory pathway of a host cell may be used.

Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57: 109-137.

Effective signal peptide coding sequences for filamentous fungal host cells are the signal peptide coding sequences obtained from the genes for Aspergillus niger neutral amylase, Aspergillus niger glucoamylase, Aspergillus oryzae TAKA amylase, Humicola insolens cellulase, Humicola insolens endoglucanase V, Humicola lanuginosa lipase, and Rhizomucor miehei aspartic proteinase.

Useful signal peptides for yeast host cells are obtained from the genes for Saccharomyces cerevisiae alpha-factor and Saccharomyces cerevisiae invertase. Other useful signal peptide coding sequences are described by Romanos et al., 1992, supra.

The control sequence may also be a propeptide coding sequence that encodes a propeptide positioned at the N-terminus of a polypeptide. The resultant polypeptide is known as a proenzyme or propolypeptide (or a zymogen in some cases). A propolypeptide is generally inactive and can be converted to an active polypeptide by catalytic or autocatalytic cleavage of the propeptide from the propolypeptide. The propeptide coding sequence may be obtained from the genes for Bacillus subtilis alkaline protease (aprE), Bacillus subtilis neutral protease (nprT), Myceliophthora thermophila laccase (WO 95/33836), Rhizomucor miehei aspartic proteinase, and Saccharomyces cerevisiae alpha-factor.

Where both signal peptide and propeptide sequences are present, the propeptide sequence is positioned next to the N-terminus of a polypeptide and the signal peptide sequence is positioned next to the N-terminus of the propeptide sequence.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound. Regulatory systems in prokaryotic systems include the lac, tac, and trp operator systems. In yeast, the ADH2 system or GAL1 system may be used. In filamentous fungi, the Aspergillus niger glucoamylase promoter, Aspergillus oryzae TAKA alpha-amylase promoter, and Aspergillus oryzae glucoamylase promoter may be used. Other examples of regulatory sequences are those that allow for gene amplification. In eukaryotic systems, these regulatory sequences include the dihydrofolate reductase gene that is amplified in the presence of methotrexate, and the metallothionein genes that are amplified with heavy metals. In these cases, the polynucleotide encoding the polypeptide would be operably linked with the regulatory sequence.

### Expression Vectors

The present invention also relates to recombinant expression vectors comprising a polynucleotide of the present invention, a promoter, and transcriptional and translational stop signals. The various nucleotide and control sequences may be joined together to produce a recombinant expression vector that may include one or more convenient restriction sites to allow for insertion or substitution of the polynucleotide encoding the polypeptide at such sites. Alternatively, the polynucleotide may be expressed by inserting the polynucleotide or a nucleic acid construct comprising the polynucleotide into an appropriate vector for expression. In creating the expression vector, the coding sequence is located in the vector so that the coding sequence is operably linked with the appropriate control sequences for expression.

The recombinant expression vector may be any vector (e.g., a plasmid or virus) that can be conveniently subjected to recombinant DNA procedures and can bring about expression of the polynucleotide. The choice of the vector will typically depend on the compatibility of the vector with the host cell into which the vector is to be introduced. The vector may be a linear or closed circular plasmid.

The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used.

The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like.

Examples of bacterial selectable markers are Bacillus licheniformis or Bacillus subtilis dal genes, or markers that confer antibiotic resistance such as ampicillin, chloramphenicol, kanamycin, neomycin, spectinomycin, or tetracycline resistance. Suitable markers for yeast host cells include, but are not limited to, ADE2, HIS3, LEU2, LYS2, MET3, TRP1, and URA3. Selectable markers for use in a filamentous fungal host cell include, but are not limited to, amdS (acetamidase), argB (ornithine carbamoyltransferase), bar (phosphinothricin acetyltransferase), hph (hygromycin phosphotransferase), niaD (nitrate reductase), pyrG (orotidine-5′-phosphate decarboxylase), sC (sulfate adenyltransferase), and trpC (anthranilate synthase), as well as equivalents thereof. Preferred for use in an Aspergillus cell are Aspergillus nidulans or Aspergillus oryzae amdS and pyrG genes and a Streptomyces hygroscopicus bar gene.

The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements should contain a sufficient number of nucleic acids, such as 100 to 10,000 base pairs, 400 to 10,000 base pairs, and 800 to 10,000 base pairs, which have a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination.

For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1060, and pAMβ1 permitting replication in Bacillus.

Examples of origins of replication for use in a yeast host cell are the 2 micron origin of replication, ARS1, ARS4, the combination of ARS1 and CEN3, and the combination of ARS4 and CEN6.

Examples of origins of replication useful in a filamentous fungal cell are AMA1 and ANS1 (Gems et al., 1991, Gene 98: 61-67; Cullen et al, 1987, Nucleic Acids Res. 15: 9163-9175; WO 00/24883). Isolation of the AMA1 gene and construction of plasmids or vectors comprising the gene can be accomplished according to the methods disclosed in WO 00/24883.

More than one copy of a polynucleotide of the present invention may be inserted into a host cell to increase production of a polypeptide. An increase in the copy number of the polynucleotide can be obtained by integrating at least one additional copy of the sequence into the host cell genome or by including an amplifiable selectable marker gene with the polynucleotide where cells containing amplified copies of the selectable marker gene, and thereby additional copies of the polynucleotide, can be selected for by cultivating the cells in the presence of the appropriate selectable agent.

The procedures used to ligate the elements described above to construct the recombinant expression vectors of the present invention are well known to one skilled in the art (see, e.g., Sambrook et al., 1989, supra).

### Host Cells

The present invention also relates to recombinant host cells, comprising a polynucleotide of the present invention operably linked to one or more control sequences that direct the production of a polypeptide of the present invention. A construct or vector comprising a polynucleotide is introduced into a host cell so that the construct or vector is maintained as a chromosomal integrant or as a self-replicating extra-chromosomal vector as described earlier. The term “host cell” encompasses any progeny of a parent cell that is not identical to the parent cell due to mutations that occur during replication. The choice of a host cell will to a large extent depend upon the gene encoding the polypeptide and its source.

The host cell may be any cell useful in the recombinant production of a polypeptide of the present invention, e.g., a prokaryote or a eukaryote.

The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma.

The bacterial host cell may be any Bacillus cell including, but not limited to, Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, and Bacillus thuringiensis cells.

The bacterial host cell may also be any Streptococcus cell including, but not limited to, Streptococcus equisimilis, Streptococcus pyogenes, Streptococcus uberis, and Streptococcus equi subsp. Zooepidemicus cells.

The bacterial host cell may also be any Streptomyces cell including, but not limited to, Streptomyces achromogenes, Streptomyces avermitilis, Streptomyces coelicolor, Streptomyces griseus, and Streptomyces lividans cells.

The introduction of DNA into a Bacillus cell may be effected by protoplast transformation (see, e.g., Chang and Cohen, 1979, Mol. Gen. Genet. 168: 111-115), competent cell transformation (see, e.g., Young and Spizizen, 1961, J. Bacteriol. 81: 823-829, or Dubnau and Davidoff-Abelson, 1971, J. Mol. Biol. 56: 209-221), electroporation (see, e.g., Shigekawa and Dower, 1988, Biotechniques 6: 742-751), or conjugation (see, e.g., Koehler and Thome, 1987, J. Bacteriol. 169: 5271-5278). The introduction of DNA into an E. coli cell may be effected by protoplast transformation (see, e.g., Hanahan, 1983, J. Mol. Biol. 166: 557-580) or electroporation (see, e.g., Dower et al., 1988, Nucleic Acids Res. 16: 6127-6145). The introduction of DNA into a Streptomyces cell may be effected by protoplast transformation, electroporation (see, e.g., Gong et al., 2004, Folia Microbiol. (Praha) 49: 399-405), conjugation (see, e.g., Mazodier et al., 1989, J. Bacteriol. 171: 3583-3585), or transduction (see, e.g., Burke et al., 2001, Proc. Natl. Acad. Sci. USA 98: 6289-6294). The introduction of DNA into a Pseudomonas cell may be effected by electroporation (see, e.g., Choi et al., 2006, J. Microbiol. Methods 64: 391-397) or conjugation (see, e.g., Pinedo and Smets, 2005, Appl. Environ. Microbiol. 71: 51-57). The introduction of DNA into a Streptococcus cell may be effected by natural competence (see, e.g., Perry and Kuramitsu, 1981, Infect. Immun. 32: 1295-1297), protoplast transformation (see, e.g., Catt and Jollick, 1991, Microbios 68: 189-207), electroporation (see, e.g., Buckley et al., 1999, Appl. Environ. Microbiol. 65: 3800-3804), or conjugation (see, e.g., Clewell, 1981, Microbiol. Rev. 45: 409-436). However, any method known in the art for introducing DNA into a host cell can be used.

The host cell may also be a eukaryote, such as a mammalian, insect, plant, or fungal cell.

The host cell may be a fungal cell. “Fungi” as used herein includes the phyla Ascomycota, Basidiomycota, Chytridiomycota, and Zygomycota as well as the Oomycota and all mitosporic fungi (as defined by Hawksworth et al., In, Ainsworth and Bisby's Dictionary of The Fungi, 8th edition, 1995, CAB International, University Press, Cambridge, UK).

The fungal host cell may be a yeast cell. “Yeast” as used herein includes ascosporogenous yeast (Endomycetales), basidiosporogenous yeast, and yeast belonging to the Fungi Imperfecti (Blastomycetes). Since the classification of yeast may change in the future, for the purposes of this invention, yeast shall be defined as described in Biology and Activities of Yeast (Skinner, Passmore, and Davenport, editors, Soc. App. Bacteriol. Symposium Series No. 9, 1980).

The yeast host cell may be a Candida, Hansenula, Kluyveromyces, Pichia, Saccharomyces, Schizosaccharomyces, or Yarrowia cell, such as a Kluyveromyces lactis, Saccharomyces carisbergensis, Saccharomyces cerevisiae, Saccharomyces diastaticus, Saccharomyces douglasii, Saccharomyces kluyveri, Saccharomyces norbensis, Saccharomyces oviformis, or Yarrowia lipolytica cell.

The fungal host cell may be a filamentous fungal cell. “Filamentous fungi” include all filamentous forms of the subdivision Eumycota and Oomycota (as defined by Hawksworth et al., 1995, supra). The filamentous fungi are generally characterized by a mycelial wall composed of chitin, cellulose, glucan, chitosan, mannan, and other complex polysaccharides. Vegetative growth is by hyphal elongation and carbon catabolism is obligately aerobic. In contrast, vegetative growth by yeasts such as Saccharomyces cerevisiae is by budding of a unicellular thallus and carbon catabolism may be fermentative.

The filamentous fungal host cell may be an Acremonium, Aspergillus, Aureobasidium, Bjerkandera, Ceriporiopsis, Chrysosporium, Coprinus, Coriolus, Cryptococcus, Filibasidium, Fusarium, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, or Trichoderma cell.

For example, the filamentous fungal host cell may be an Aspergillus awamori, Aspergillus foetidus, Aspergillus fumigatus, Aspergillus japonicus, Aspergillus nidulans, Aspergillus niger, Aspergillus oryzae, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporium inops, Chrysosporium keratinophilum, Chrysosporium lucknowense, Chrysosporium merdarium, Chrysosporium pannicola, Chrysosporium queenslandicum, Chrysosporium tropicum, Chrysosporium zonaturn, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, or Trichoderma viride cell.

Fungal cells may be transformed by a process involving protoplast formation, transformation of the protoplasts, and regeneration of the cell wall in a manner known per se. Suitable procedures for transformation of Aspergillus and Trichoderma host cells are described in EP 238023, Yelton et al., 1984, Proc. Natl. Acad. Sci. USA 81: 1470-1474, and Christensen et al, 1988, Bio/Technology 6: 1419-1422. Suitable methods for transforming Fusarium species are described by Malardier et al., 1989, Gene 78: 147-156, and WO 96/00787. Yeast may be transformed using the procedures described by Becker and Guarente, In Abelson, J. N. and Simon, M. I., editors, Guide to Yeast Genetics and Molecular Biology, Methods in Enzymology, Volume 194, pp 182-187, Academic Press, Inc., New York; Ito et al., 1983, J. Bacteriol. 153: 163; and Hinnen et al., 1978, Proc. Natl. Acad. Sci. USA 75: 1920.

### Methods of Production

The present invention also relates to methods of producing a polypeptide of the present invention, comprising (a) cultivating a cell, which in its wild-type form produces the polypeptide, under conditions conducive for production of the polypeptide; and (b) recovering the polypeptide. In a preferred aspect, the cell is a Aspergillus or a Penicillium cell. In a more preferred aspect, the cell is a Aspergillus aculeatus cell or Penicillium pinophilum.

The present invention also relates to methods of producing a polypeptide of the present invention, comprising (a) cultivating a recombinant host cell of the present invention under conditions conducive for production of the polypeptide; and (b) optionally recovering the polypeptide.

The host cells are cultivated in a nutrient medium suitable for production of the polypeptide using methods known in the art. For example, the cell may be cultivated by shake flask cultivation, or small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection). If the polypeptide is secreted into the nutrient medium, the polypeptide can be recovered directly from the medium. If the polypeptide is not secreted, it can be recovered from cell lysates.

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide.

The polypeptide may be recovered using methods known in the art. For example, the polypeptide may be recovered from the nutrient medium by conventional procedures including, but not limited to, collection, centrifugation, filtration, extraction, spray-drying, evaporation, or precipitation.

The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain substantially pure polypeptides.

In an alternative aspect, the polypeptide is not recovered, but rather a host cell of the present invention expressing the polypeptide is used as a source of the polypeptide.

### Plants

The present invention also relates to isolated plants, e.g., a transgenic plant, plant part, or plant cell, comprising a polynucleotide of the present invention so as to express and produce a polypeptide or domain in recoverable quantities. The polypeptide or domain may be recovered from the plant or plant part. Alternatively, the plant or plant part containing the polypeptide or domain may be used as such for improving the quality of a food or feed, e.g., improving nutritional value, palatability, and rheological properties, or to destroy an antinutritive factor.

The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn).

Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana.

Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats.

Also included within the scope of the present invention are the progeny of such plants, plant parts, and plant cells.

The transgenic plant or plant cell expressing the polypeptide or domain may be constructed in accordance with methods known in the art. In short, the plant or plant cell is constructed by incorporating one or more expression constructs encoding the polypeptide or domain into the plant host genome or chloroplast genome and propagating the resulting modified plant or plant cell into a transgenic plant or plant cell.

The expression construct is conveniently a nucleic acid construct that comprises a polynucleotide encoding a polypeptide or domain operably linked with appropriate regulatory sequences required for expression of the polynucleotide in the plant or plant part of choice. Furthermore, the expression construct may comprise a selectable marker useful for identifying plant cells into which the expression construct has been integrated and DNA sequences necessary for introduction of the construct into the plant in question (the latter depends on the DNA introduction method to be used).

The choice of regulatory sequences, such as promoter and terminator sequences and optionally signal or transit sequences, is determined, for example, on the basis of when, where, and how the polypeptide or domain is desired to be expressed. For instance, the expression of the gene encoding a polypeptide or domain may be constitutive or inducible, or may be developmental, stage or tissue specific, and the gene product may be targeted to a specific tissue or plant part such as seeds or leaves. Regulatory sequences are, for example, described by Tague et al., 1988, Plant Physiology 86: 506.

For constitutive expression, the 35S-CaMV, the maize ubiquitin 1, or the rice actin 1 promoter may be used (Franck et al, 1980, Cell 21: 285-294; Christensen et al, 1992, Plant Mol. Biol. 18: 675-689; Zhang et al., 1991, Plant Cell 3: 1155-1165). Organ-specific promoters may be, for example, a promoter from storage sink tissues such as seeds, potato tubers, and fruits (Edwards and Coruzzi, 1990, Ann. Rev. Genet. 24: 275-303), or from metabolic sink tissues such as meristems (Ito et al., 1994, Plant Mol. Biol. 24: 863-878), a seed specific promoter such as the glutelin, prolamin, globulin, or albumin promoter from rice (Wu et al., 1998, Plant Cell Physiol. 39: 885-889), a Vicia faba promoter from the legumin B4 and the unknown seed protein gene from Vicia faba (Conrad et al., 1998, J. Plant Physiol. 152: 708-711), a promoter from a seed oil body protein (Chen et al., 1998, Plant Cell Physiol. 39: 935-941), the storage protein napA promoter from Brassica napus, or any other seed specific promoter known in the art, e.g., as described in WO 91/14772. Furthermore, the promoter may be a leaf specific promoter such as the rbcs promoter from rice or tomato (Kyozuka et al, 1993, Plant Physiol. 102: 991-1000), the chlorella virus adenine methyltransferase gene promoter (Mitra and Higgins, 1994, Plant Mol. Biol. 26: 85-93), the aldP gene promoter from rice (Kagaya et al., 1995, Mol. Gen. Genet. 248: 668-674), or a wound inducible promoter such as the potato pin2 promoter (Xu et al., 1993, Plant Mol. Biol. 22: 573-588). Likewise, the promoter may be induced by abiotic treatments such as temperature, drought, or alterations in salinity or induced by exogenously applied substances that activate the promoter, e.g., ethanol, oestrogens, plant hormones such as ethylene, abscisic acid, and gibberellic acid, and heavy metals.

A promoter enhancer element may also be used to achieve higher expression of a polypeptide or domain in the plant. For instance, the promoter enhancer element may be an intron that is placed between the promoter and the polynucleotide encoding a polypeptide or domain. For instance, Xu et al., 1993, supra, disclose the use of the first intron of the rice actin 1 gene to enhance expression.

The selectable marker gene and any other parts of the expression construct may be chosen from those available in the art.

The nucleic acid construct is incorporated into the plant genome according to conventional techniques known in the art, including Agrobacterium-mediated transformation, virus-mediated transformation, microinjection, particle bombardment, biolistic transformation, and electroporation (Gasser et al., 1990, Science 244: 1293; Potrykus, 1990, Bio/Technology 8: 535; Shimamoto et al., 1989, Nature 338: 274).

Agrobacterium tumefaciens-mediated gene transfer is a method for generating transgenic dicots (for a review, see Hooykas and Schilperoort, 1992, Plant Mol. Biol. 19: 15-38) and for transforming monocots, although other transformation methods may be used for these plants. A method for generating transgenic monocots is particle bombardment (microscopic gold or tungsten particles coated with the transforming DNA) of embryonic calli or developing embryos (Christou, 1992, Plant J. 2: 275-281; Shimamoto, 1994, Curr. Opin. Biotechnol. 5: 158-162; Vasil et al, 1992, Bio/Technology 10: 667-674). An alternative method for transformation of monocots is based on protoplast transformation as described by Omirulleh et al., 1993, Plant Mol. Biol. 21: 415-428. Additional transformation methods include those described in U.S. Pat. Nos. 6,395,966 and 7,151,204 (both of which are herein incorporated by reference in their entirety).

Following transformation, the transformants having incorporated the expression construct are selected and regenerated into whole plants according to methods well known in the art. Often the transformation procedure is designed for the selective elimination of selection genes either during regeneration or in the following generations by using, for example, co-transformation with two separate T-DNA constructs or site specific excision of the selection gene by a specific recombinase.

In addition to direct transformation of a particular plant genotype with a construct of the present invention, transgenic plants may be made by crossing a plant having the construct to a second plant lacking the construct. For example, a construct encoding a polypeptide or domain can be introduced into a particular plant variety by crossing, without the need for ever directly transforming a plant of that given variety. Therefore, the present invention encompasses not only a plant directly regenerated from cells which have been transformed in accordance with the present invention, but also the progeny of such plants. As used herein, progeny may refer to the offspring of any generation of a parent plant prepared in accordance with the present invention. Such progeny may include a DNA construct prepared in accordance with the present invention. Crossing results in the introduction of a transgene into a plant line by cross pollinating a starting line with a donor plant line. Non-limiting examples of such steps are described in U.S. Pat. No. 7,151,204.

Plants may be generated through a process of backcross conversion. For example, plants include plants referred to as a backcross converted genotype, line, inbred, or hybrid.

Genetic markers may be used to assist in the introgression of one or more transgenes of the invention from one genetic background into another. Marker assisted selection offers advantages relative to conventional breeding in that it can be used to avoid errors caused by phenotypic variations. Further, genetic markers may provide data regarding the relative degree of elite germplasm in the individual progeny of a particular cross. For example, when a plant with a desired trait which otherwise has a non-agronomically desirable genetic background is crossed to an elite parent, genetic markers may be used to select progeny which not only possess the trait of interest, but also have a relatively large proportion of the desired germplasm. In this way, the number of generations required to introgress one or more traits into a particular genetic background is minimized.

The present invention also relates to methods of producing a polypeptide or domain of the present invention comprising (a) cultivating a transgenic plant or a plant cell comprising a polynucleotide encoding the polypeptide or domain under conditions conducive for production of the polypeptide or domain; and (b) recovering the polypeptide or domain.

### Removal or Reduction of Berberine Bridge Enzyme Like (BBEL) Activity

The present invention also relates to methods of producing a mutant of a parent cell, which comprises disrupting or deleting a polynucleotide, or a portion thereof, encoding a polypeptide of the present invention, which results in the mutant cell producing less of the polypeptide than the parent cell when cultivated under the same conditions.

The mutant cell may be constructed by reducing or eliminating expression of the polynucleotide using methods well known in the art, for example, insertions, disruptions, replacements, or deletions. In a preferred aspect, the polynucleotide is inactivated. The polynucleotide to be modified or inactivated may be, for example, the coding region or a part thereof essential for activity, or a regulatory element required for expression of the coding region. An example of such a regulatory or control sequence may be a promoter sequence or a functional part thereof, i.e., a part that is sufficient for affecting expression of the polynucleotide. Other control sequences for possible modification include, but are not limited to, a leader, polyadenylation sequence, propeptide sequence, signal peptide sequence, transcription terminator, and transcriptional activator.

Modification or inactivation of the polynucleotide may be performed by subjecting the parent cell to mutagenesis and selecting for mutant cells in which expression of the polynucleotide has been reduced or eliminated. The mutagenesis, which may be specific or random, may be performed, for example, by use of a suitable physical or chemical mutagenizing agent, by use of a suitable oligonucleotide, or by subjecting the DNA sequence to PCR generated mutagenesis. Furthermore, the mutagenesis may be performed by use of any combination of these mutagenizing agents.

Examples of a physical or chemical mutagenizing agent suitable for the present purpose include ultraviolet (UV) irradiation, hydroxylamine, N-methyl-N′-nitro-N-nitrosoguanidine (MNNG), O-methyl hydroxylamine, nitrous acid, ethyl methane sulphonate (EMS), sodium bisulphite, formic acid, and nucleotide analogues.

When such agents are used, the mutagenesis is typically performed by incubating the parent cell to be mutagenized in the presence of the mutagenizing agent of choice under suitable conditions, and screening and/or selecting for mutant cells exhibiting reduced or no expression of the gene.

Modification or inactivation of the polynucleotide may be accomplished by insertion, substitution, or deletion of one or more nucleotides in the gene or a regulatory element required for transcription or translation thereof. For example, nucleotides may be inserted or removed so as to result in the introduction of a stop codon, the removal of the start codon, or a change in the open reading frame. Such modification or inactivation may be accomplished by site-directed mutagenesis or PCR generated mutagenesis in accordance with methods known in the art. Although, in principle, the modification may be performed in vivo, i.e., directly on the cell expressing the polynucleotide to be modified, it is preferred that the modification be performed in vitro as exemplified below.

An example of a convenient way to eliminate or reduce expression of a polynucleotide is based on techniques of gene replacement, gene deletion, or gene disruption. For example, in the gene disruption method, a nucleic acid sequence corresponding to the endogenous polynucleotide is mutagenized in vitro to produce a defective nucleic acid sequence that is then transformed into the parent cell to produce a defective gene. By homologous recombination, the defective nucleic acid sequence replaces the endogenous polynucleotide. It may be desirable that the defective polynucleotide also encodes a marker that may be used for selection of transformants in which the polynucleotide has been modified or destroyed. In an aspect, the polynucleotide is disrupted with a selectable marker such as those described herein.

The present invention also relates to methods of inhibiting the expression of a polypeptide having Berberine Bridge Enzyme Like activity in a cell, comprising administering to the cell or expressing in the cell a double-stranded RNA (dsRNA) molecule, wherein the dsRNA comprises a subsequence of a polynucleotide of the present invention. In a preferred aspect, the dsRNA is about 15, 16, 17, 18, 19, 20, 21, 22, 23, 24, 25 or more duplex nucleotides in length.

The dsRNA is preferably a small interfering RNA (siRNA) or a micro RNA (miRNA). In a preferred aspect, the dsRNA is small interfering RNA for inhibiting transcription. In another preferred aspect, the dsRNA is micro RNA for inhibiting translation.

The present invention also relates to such double-stranded RNA (dsRNA) molecules, comprising a portion of the mature polypeptide coding sequence of SEQ ID NO: 1 for inhibiting expression of the polypeptide in a cell. While the present invention is not limited by any particular mechanism of action, the dsRNA can enter a cell and cause the degradation of a single-stranded RNA (ssRNA) of similar or identical sequences, including endogenous mRNAs. When a cell is exposed to dsRNA, mRNA from the homologous gene is selectively degraded by a process called RNA interference (RNAi).

The dsRNAs of the present invention can be used in gene-silencing. In one aspect, the invention provides methods to selectively degrade RNA using a dsRNAi of the present invention. The process may be practiced in vitro, ex vivo or in vivo. In one aspect, the dsRNA molecules can be used to generate a loss-of-function mutation in a cell, an organ or an animal. Methods for making and using dsRNA molecules to selectively degrade RNA are well known in the art; see, for example, U.S. Pat. Nos. 6,489,127; 6,506,559; 6,511,824; and 6,515,109.

The present invention further relates to a mutant cell of a parent cell that comprises a disruption or deletion of a polynucleotide encoding the polypeptide or a control sequence thereof or a silenced gene encoding the polypeptide, which results in the mutant cell producing less of the polypeptide or no polypeptide compared to the parent cell.

The polypeptide-deficient mutant cells are particularly useful as host cells for expression of native and heterologous polypeptides. Therefore, the present invention further relates to methods of producing a native or heterologous polypeptide, comprising (a) cultivating the mutant cell under conditions conducive for production of the polypeptide; and (b) recovering the polypeptide. The term “heterologous polypeptides” means polypeptides that are not native to the host cell, e.g., a variant of a native protein. The host cell may comprise more than one copy of a polynucleotide encoding the native or heterologous polypeptide.

The methods used for cultivation and purification of the product of interest may be performed by methods known in the art.

The methods of the present invention for producing an essentially berberine bridge enzyme-free product is of particular interest in the production of eukaryotic polypeptides, in particular fungal proteins such as enzymes. The berberine bridge enzyme-deficient cells may also be used to express heterologous proteins of pharmaceutical interest such as hormones, growth factors, receptors, and the like. The term “eukaryotic polypeptides” includes not only native polypeptides, but also those polypeptides, e.g., enzymes, which have been modified by amino acid substitutions, deletions or additions, or other such modifications to enhance activity, thermostability, pH tolerance and the like.

In a further aspect, the present invention relates to a protein product essentially free from Berberine Bridge Enzyme Like activity that is produced by a method of the present invention.

### Compositions

The present invention also relates to a enzyme composition comprising a cellulolytic preparation and an Berberine Bridge Enzyme Like (BBEL) enzyme.

The present invention also relates to enzyme compositions comprising a cellulolytic preparation optionally derived from Trichoderma reesei, Humicola insolens or Chrysosporium lucknowense and a Berberine Bridge Enzyme Like (BBEL) enzyme. The cellulotic preparation can comprise one or more (several) enzymatic activities, these can be selected from the group consisting of a GH61 polypeptide having cellulolytic enhancing activity, a beta-glucosidase, a xylanase, a beta-xylosidase, a CBHI and a CBHII.

The enzyme composition of present invention may further comprise: a cellulolytic preparation comprising a GH61 polypeptide having cellulolytic enhancing activity and a beta-glucosidase and/or a cellulolytic preparation comprising a GH61 polypeptide having cellulolytic enhancing activity, a beta-glucosidase, and a xylanase and/or a cellulolytic preparation comprising a GH61 polypeptide having cellulolytic enhancing activity, a beta-glucosidase, a xylanase and a beta-xylosidase an/or a cellulolytic preparation comprising a GH61 polypeptide having cellulolytic enhancing activity, a beta-glucosidase, a xylanase, a beta-xylosidase, and a CBHI and/or a cellulolytic preparation comprising a GH61 polypeptide having cellulolytic enhancing activity, a beta-glucosidase, a xylanase, a beta-xylosidase, a CBHI and a CBHII.

The cellulolytic preparation may comprise a beta-glucosidase, preferably one derived from a strain of the genus Aspergillus, such as Aspergillus oryzae, such as the one disclosed in WO 2002/095014 or the fusion protein having beta-glucosidase activity disclosed in WO 2008/057637, or Aspergillus fumigates, such as such as one disclosed in WO 2005/047499 or an Aspergillus fumigatus beta-glucosidase variant disclosed in co-pending U.S. provisional application No. 61/388,997; or a strain of the genus a strain Penicillium, such as a strain of the Penicillium brasilianum disclosed in WO 2007/019442, or a strain of the genus Trichoderma, such as a strain of Trichoderma reesei and optionaly a GH61 polypeptide having cellulolytic enhancing activity such as one derived from the genus Thermoascus, such as a strain of Thermoascus aurantiacus, such as the one described in WO 2005/074656 as SEQ ID NO: 2; or one derived from the genus Thielavia, such as a strain of Thielavia terrestris, such as the one described in WO 2005/074647 as SEQ ID NO: 7 and SEQ ID NO: 8; or one derived from a strain of Aspergillus, such as a strain of Aspergillus fumigates, such as the one described in WO 2010/138754 as SEQ ID NO: 1 and SEQ ID NO: 2; or one derived from a strain derived from Penicillium, such as a strain of Penicillium emersonii, such as the one disclosed in WO 2011/041397.

In a related aspect, the cellulolytic preparation comprises a xylanase, preferably a GH10 xylanase, such as one derived from a strain of the genus Aspergillus, such as a strain from Aspergillus fumigatus, such as the one disclosed as Xyl III in WO 2006/078256, or Aspergillus aculeatus, such as the one disclosed in WO 94/21785 as SEQ ID NO: 5 (Xyl II) additionally or alternatively, the cellulolytic preparation comprises a beta-xylosidase, such as one derived from a strain of the genus Aspergillus, such as a strain of Aspergillus fumigatus, such as the one disclosed in co-pending US provisional #61/526833, or derived from a strain of Trichoderma, such as a strain of Trichoderma reesei, such as the mature polypeptide of SEQ ID NO: 58 in WO 2011/057140.

The enzyme composition of present invention may further comprise a cellulolytic preparation comprising a cellobiohydrolase I (CBH I), such as one derived from a strain of the genus Aspergillus, such as a strain of Aspergillus fumigatus, such as the Cel7a CBHI disclosed in SEQ ID NO: 2 in WO 2011/057140, or a strain of the genus Trichoderma, such as a strain of Trichoderma reesei and/or a cellulolytic preparation comprising a cellobiohydrolase II (CBH II, such as one derived from a strain of the genus Aspergillus, such as a strain of Aspergillus fumigates; or a strain of the genus Trichoderma, such as Trichoderma reesei, or a strain of the genus Thielavia, such as a strain of Thielavia terrestris, such as cellobiohydrolase II CEL6A from Thielavia terrestris.

Further, the enzyme composition of present invention comprise a Berberine Bridge Enzyme Like (BBEL) derived from a strain of the genus Aspergillus, such as a strain of Aspergillus aculeatus, such as Aspergillus aculeatus CBS 172.66 or a Berberine Bridge Enzyme Like (BBEL) having at least 80% sequence identity to the mature polypeptide of any of SEQ ID NO: 6, SEQ ID NO: 8 or SEQ ID NO: 10, at least 75% sequence identity to the mature polypeptide of any of SEQ ID NO: 12 or SEQ ID NO:14, or at least 60% sequence identity to the mature polypeptide of SEQ ID NO: 16 herein. Additionally or alternatively, the enzyme composition of present invention may comprise a Berberine Bridge Enzyme Like (BBEL) which is derived from a strain of the genus Penicillium, such as a strain of Penicillium pinophilum or an acetylxylan esterase having at least 95% sequence identity to the mature polypeptide of SEQ ID NO: 2 or at least 90% sequence identity to the mature polypeptide of SEQ ID NO: 4 herein.

Yet another aspect of the present invention, relates to an enzyme composition wherein the cellulolytic preparation is a Trichoderma reesei cellulolytic preparation, further comprising Thermoascus aurantiacus GH61A polypeptide having cellulolytic enhancing activity (SEQ ID NO: 2 in WO 2005/074656), Aspergillus oryzae beta-glucosidase fusion protein (SEQ ID NOs: 75 and 76 in WO 2008/057637), and Aspergillus aculeatus xylanase (Xyl II in WO 94/21785), or wherein the cellulolytic preparation is a Trichoderma reesei cellulolytic preparation, further comprising Thermoascus aurantiacus GH61A polypeptide having cellulolytic enhancing activity (SEQ ID NO: 2 in WO 2005/074656), Aspergillus fumigatus beta-glucosidase (SEQ ID NO: 2 of WO 2005/047499) and Aspergillus aculeatus xylanase (Xyl II disclosed in WO 94/21785) or wherein the cellulolytic preparation is a Trichoderma reesei cellulolytic preparation further comprising Thermoascus aurantiacus GH61A polypeptide having cellulolytic enhancing activity (SEQ ID NO: 2 in WO 2005/074656), Aspergillus fumigatus beta-glucosidase (SEQ ID NO: 2 of WO 2005/047499) and Aspergillus aculeatus xylanase (Xyl II disclosed in WO 94/21785) and the acetylxylan esterase (AXE) is the one derived from Thielavia terrestris disclosed in WO 2009/042846 as SEQ ID NO: 2 or SEQ ID NO: 1 herein or an acetylxylan esterase having at least 80%, such as at least 85%, such as at least 90%, preferably at least 95%, such as at least 96%, such as at least 97%, such as at least 98%, such as at least 99% identity to SEQ ID NO: 2 in WO 2009/042846 SEQ ID NO: 1 herein or wherein the cellulolytic preparation is a Trichoderma reesei cellulolytic preparation further comprising Penicillium emersonii GH61A polypeptide having cellulolytic enhancing activity disclosed in WO 2011/041397, Aspergillus fumigatus beta-glucosidase (SEQ ID NO: 2 of WO 2005/047499) and Aspergillus fumigatus xylanase (Xyl III in WO 2006/078256) and the acetylxylan esterase (AXE) is the one derived from Thielavia terrestris disclosed in WO 2009/042846 as SEQ ID NO: 2 or SEQ ID NO: 1 or an acetylxylan esterase having at least 80%, such as at least 85%, such as at least 90%, preferably at least 95%, such as at least 96%, such as at least 97%, such as at least 98%, such as at least 99% identity to SEQ ID NO: 2 in WO 2009/042846 SEQ ID NO: 1.

### Methods of Processing Cellulosic Material

The present invention also relates to methods for degrading or converting a cellulosic material, comprising: treating the cellulosic material with an enzyme composition in the presence of a polypeptide having Berberine Bridge Enzyme Like activity of the present invention. In on aspect the invention relates to a method of hydrolyzing cellulosic material, comprising subjecting the cellulosic material to a cellulolytic preparation and a berberine bridge enzymes like (BBEL) enzyme. In a preferred aspect, the method further comprises recovering the degraded or converted cellulosic material.

The present invention also relates to methods of producing a fermentation product, comprising: (a) saccharifying a cellulosic material with an enzyme composition in the presence of a polypeptide having Berberine Bridge Enzyme Like activity of the present invention; (b) fermenting the saccharified cellulosic material with one or more (several) fermenting microorganisms to produce the fermentation product; and (c) recovering the fermentation product from the fermentation. The present invention also relates to methods of fermenting a cellulosic material, comprising: fermenting the cellulosic material with one or more (several) fermenting microorganisms, wherein the cellulosic material is saccharified with an enzyme composition in the presence of a polypeptide having Berberine Bridge Enzyme Like activity of the present invention. In a preferred aspect, the fermenting of the cellulosic material produces a fermentation product. In another preferred aspect, the method further comprises recovering the fermentation product from the fermentation.

In yet a related aspect of the present invention relates to a process of producing a fermentation product from cellulosic material, comprises: (a) hydrolyzing said cellulosic material by subjecting the material to an enzyme composition according to the present invention or to a hydrolysis method according to the present invention; (b) fermenting using a fermenting organism; and (c) optionally recovering the fermentation product.

The methods of the present invention can be used to saccharify a cellulosic material to fermentable sugars and convert the fermentable sugars to many useful substances, e.g., fuel, potable ethanol, and/or fermentation products (e.g., acids, alcohols, ketones, gases, and the like). The production of a desired fermentation product from cellulosic material typically involves pretreatment, enzymatic hydrolysis (saccharification), and fermentation.

The processing of cellulosic material according to the present invention can be accomplished using processes conventional in the art. Moreover, the methods of the present invention can be implemented using any conventional biomass processing apparatus configured to operate in accordance with the invention.

Hydrolysis (saccharification) and fermentation, separate or simultaneous, include, but are not limited to, separate hydrolysis and fermentation (SHF); simultaneous saccharification and fermentation (SSF); simultaneous saccharification and cofermentation (SSCF); hybrid hydrolysis and fermentation (HHF); separate hydrolysis and co-fermentation (SHCF); hybrid hydrolysis and co-fermentation (HHCF); and direct microbial conversion (DMC). SHF uses separate process steps to first enzymatically hydrolyze cellulosic material to fermentable sugars, e.g., glucose, cellobiose, cellotriose, and pentose sugars, and then ferment the fermentable sugars to ethanol. In SSF, the enzymatic hydrolysis of cellulosic material and the fermentation of sugars to ethanol are combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF involves the cofermentation of multiple sugars (Sheehan, J., and Himmel, M., 1999, Enzymes, energy and the environment: A strategic perspective on the U.S. Department of Energy's research and development activities for bioethanol, Biotechnol. Prog. 15: 817-827). HHF involves a separate hydrolysis step, and in addition a simultaneous saccharification and hydrolysis step, which can be carried out in the same reactor. The steps in an HHF process can be carried out at different temperatures, i.e., high temperature enzymatic saccharification followed by SSF at a lower temperature that the fermentation strain can tolerate. DMC combines all three processes (enzyme production, hydrolysis, and fermentation) in one or more (several) steps where the same organism is used to produce the enzymes for conversion of the cellulosic material to fermentable sugars and to convert the fermentable sugars into a final product (Lynd, L. R., Weimer, P. J., van Zyl, W. H., and Pretorius, I. S., 2002, Microbial cellulose utilization: Fundamentals and biotechnology, Microbiol. Mol. Biol. Reviews 66: 506-577). It is understood herein that any method known in the art comprising pretreatment, enzymatic hydrolysis (saccharification), fermentation, or a combination thereof, can be used in the practicing the methods of the present invention.

A conventional apparatus can include a fed-batch stirred reactor, a batch stirred reactor, a continuous flow stirred reactor with ultrafiltration, and/or a continuous plug-flow column reactor (Fernanda de Castilhos Corazza, Flávio Faria de Moraes, Gisella Maria Zanin and Ivo Neitzel, 2003, Optimal control in fed-batch reactor for the cellobiose hydrolysis, Acta Scientiarum. Technology 25: 33-38; Gusakov, A. V., and Sinitsyn, A. P., 1985, Kinetics of the enzymatic hydrolysis of cellulose: 1. A mathematical model for a batch reactor process, Enz. Microb. Technol. 7: 346-352), an attrition reactor (Ryu, S. K., and Lee, J. M., 1983, Bioconversion of waste cellulose by using an attrition bioreactor, Biotechnol. Bioeng. 25: 53-65), or a reactor with intensive stirring induced by an electromagnetic field (Gusakov, A. V., Sinitsyn, A. P., Davydkin, I. Y., Davydkin, V. Y., Protas, 0. V., 1996, Enhancement of enzymatic cellulose hydrolysis using a novel type of bioreactor with intensive stirring induced by electromagnetic field, Appl. Biochem. Biotechnol. 56: 141-153). Additional reactor types include: fluidized bed, upflow blanket, immobilized, and extruder type reactors for hydrolysis and/or fermentation.

Pretreatment.

In practicing the methods of the present invention, any pretreatment process known in the art can be used to disrupt plant cell wall components of cellulosic material (Chandra et al., 2007, Substrate pretreatment: The key to effective enzymatic hydrolysis of lignocellulosics? Adv. Biochem. Engin./Biotechnol. 108: 67-93; Galbe and Zacchi, 2007, Pretreatment of lignocellulosic materials for efficient bioethanol production, Adv. Biochem. Engin./Biotechnol 108: 41-65; Hendriks and Zeeman, 2009, Pretreatments to enhance the digestibility of lignocellulosic biomass, Bioresource Technol. 100: 10-18; Mosier et al., 2005, Features of promising technologies for pretreatment of lignocellulosic biomass, Bioresource Technol. 96: 673-686; Taherzadeh and Karimi, 2008, Pretreatment of lignocellulosic wastes to improve ethanol and biogas production: A review, Int. J. of Mol. Sci. 9: 1621-1651; Yang and Wyman, 2008, Pretreatment: the key to unlocking low-cost cellulosic ethanol, Biofuels Bioproducts and Biorefining-Biofpr. 2: 26-40).

The cellulosic material can also be subjected to particle size reduction, pre-soaking, wetting, washing, or conditioning prior to pretreatment using methods known in the art.

Conventional pretreatments include, but are not limited to, steam pretreatment (with or without explosion), dilute acid pretreatment, hot water pretreatment, alkaline pretreatment, lime pretreatment, wet oxidation, wet explosion, ammonia fiber explosion, organosolv pretreatment, and biological pretreatment. Additional pretreatments include ammonia percolation, ultrasound, electroporation, microwave, supercritical CO₂, supercritical H₂O, ozone, and gamma irradiation pretreatments.

The cellulosic material can be pretreated before hydrolysis and/or fermentation. Pretreatment is preferably performed prior to the hydrolysis. Alternatively, the pretreatment can be carried out simultaneously with enzyme hydrolysis to release fermentable sugars, such as glucose, xylose, and/or cellobiose. In most cases the pretreatment step itself results in some conversion of biomass to fermentable sugars (even in absence of enzymes).

Steam Pretreatment. In steam pretreatment, cellulosic material is heated to disrupt the plant cell wall components, including lignin, hemicellulose, and cellulose to make the cellulose and other fractions, e.g., hemicellulose, accessible to enzymes. Cellulosic material is passed to or through a reaction vessel where steam is injected to increase the temperature to the required temperature and pressure and is retained therein for the desired reaction time. Steam pretreatment is preferably done at 140-230° C., more preferably 160-200° C., and most preferably 170-190° C., where the optimal temperature range depends on any addition of a chemical catalyst. Residence time for the steam pretreatment is preferably 1-15 minutes, more preferably 3-12 minutes, and most preferably 4-10 minutes, where the optimal residence time depends on temperature range and any addition of a chemical catalyst. Steam pretreatment allows for relatively high solids loadings, so that cellulosic material is generally only moist during the pretreatment. The steam pretreatment is often combined with an explosive discharge of the material after the pretreatment, which is known as steam explosion, that is, rapid flashing to atmospheric pressure and turbulent flow of the material to increase the accessible surface area by fragmentation (Duff and Murray, 1996, Bioresource Technology 855: 1-33; Galbe and Zacchi, 2002, Appl. Microbiol. Biotechnol. 59: 618-628; U.S. Patent Application No. 20020164730). During steam pretreatment, hemicellulose acetyl groups are cleaved and the resulting acid autocatalyzes partial hydrolysis of the hemicellulose to monosaccharides and oligosaccharides. Lignin is removed to only a limited extent.

A catalyst such as H₂SO₄ or SO₂ (typically 0.3 to 3% w/w) is often added prior to steam pretreatment, which decreases the time and temperature, increases the recovery, and improves enzymatic hydrolysis (Ballesteros et al., 2006, Appl. Biochem. Biotechnol. 129-132: 496-508; Varga et al., 2004, Appl. Biochem. Biotechnol. 113-116: 509-523; Sassner et al., 2006, Enzyme Microb. Technol. 39: 756-762).

Chemical Pretreatment: The term “chemical treatment” refers to any chemical pretreatment that promotes the separation and/or release of cellulose, hemicellulose, and/or lignin. Examples of suitable chemical pretreatment processes include, for example, dilute acid pretreatment, lime pretreatment, wet oxidation, ammonia fiber/freeze explosion (AFEX), ammonia percolation (APR), and organosolv pretreatments.

In dilute acid pretreatment, cellulosic material is mixed with dilute acid, typically H₂SO₄, and water to form a slurry, heated by steam to the desired temperature, and after a residence time flashed to atmospheric pressure. The dilute acid pretreatment can be performed with a number of reactor designs, e.g., plug-flow reactors, counter-current reactors, or continuous counter-current shrinking bed reactors (Duff and Murray, 1996, supra; Schell et al., 2004, Bioresource Technol. 91: 179-188; Lee et al, 1999, Adv. Biochem. Eng. Biotechnol. 65: 93-115).

Several methods of pretreatment under alkaline conditions can also be used. These alkaline pretreatments include, but are not limited to, lime pretreatment, wet oxidation, ammonia percolation (APR), and ammonia fiber/freeze explosion (AFEX).

Lime pretreatment is performed with calcium carbonate, sodium hydroxide, or ammonia at low temperatures of 85-150° C. and residence times from 1 hour to several days (Wyman et al, 2005, Bioresource Technol 96: 1959-1966; Mosier et al., 2005, Bioresource Technol. 96: 673-686). WO 2006/110891, WO 2006/11899, WO 2006/11900, and WO 2006/110901 disclose pretreatment methods using ammonia.

Wet oxidation is a thermal pretreatment performed typically at 180-200° C. for 5-15 minutes with addition of an oxidative agent such as hydrogen peroxide or over-pressure of oxygen (Schmidt and Thomsen, 1998, Bioresource Technol. 64: 139-151; Palonen et al, 2004, Appl. Biochem. Biotechnol. 117: 1-17; Varga et al, 2004, Biotechnol. Bioeng. 88: 567-574; Martin et al., 2006, J. Chem. Technol. Biotechnol 81: 1669-1677). The pretreatment is performed at preferably 1-40% dry matter, more preferably 2-30% dry matter, and most preferably 5-20% dry matter, and often the initial pH is increased by the addition of alkali such as sodium carbonate.

A modification of the wet oxidation pretreatment method, known as wet explosion (combination of wet oxidation and steam explosion), can handle dry matter up to 30%. In wet explosion, the oxidizing agent is introduced during pretreatment after a certain residence time. The pretreatment is then ended by flashing to atmospheric pressure (WO 2006/032282).

Ammonia fiber explosion (AFEX) involves treating cellulosic material with liquid or gaseous ammonia at moderate temperatures such as 90-100° C. and high pressure such as 17-20 bar for 5-10 minutes, where the dry matter content can be as high as 60% (Gollapalli et al., 2002, Appl. Biochem. Biotechnol. 98: 23-35; Chundawat et al., 2007, Biotechnol. Bioeng. 96: 219-231; Alizadeh et al, 2005, Appl. Biochem. Biotechnol. 121: 1133-1141; Teymouri et al, 2005, Bioresource Technol. 96: 2014-2018). AFEX pretreatment results in the depolymerization of cellulose and partial hydrolysis of hemicellulose. Lignin-carbohydrate complexes are cleaved.

Organosolv pretreatment delignifies cellulosic material by extraction using aqueous ethanol (40-60% ethanol) at 160-200° C. for 30-60 minutes (Pan et al, 2005, Biotechnol. Bioeng. 90: 473-481; Pan et al., 2006, Biotechnol. Bioeng. 94: 851-861; Kurabi et al, 2005, Appl. Biochem. Biotechnol. 121: 219-230). Sulphuric acid is usually added as a catalyst. In organosolv pretreatment, the majority of hemicellulose is removed.

Other examples of suitable pretreatment methods are described by Schell et al, 2003, Appl. Biochem. and Biotechnol. Vol. 105-108, p. 69-85, and Mosier et al., 2005, Bioresource Technology 96: 673-686, and U.S. Published Application 2002/0164730.

In one aspect, the chemical pretreatment is preferably carried out as an acid treatment, and more preferably as a continuous dilute and/or mild acid treatment. The acid is typically sulfuric acid, but other acids can also be used, such as acetic acid, citric acid, nitric acid, phosphoric acid, tartaric acid, succinic acid, hydrogen chloride, or mixtures thereof. Mild acid treatment is conducted in the pH range of preferably 1-5, more preferably 1-4, and most preferably 1-3. In one aspect, the acid concentration is in the range from preferably 0.01 to 20 wt % acid, more preferably 0.05 to 10 wt % acid, even more preferably 0.1 to 5 wt % acid, and most preferably 0.2 to 2.0 wt % acid. The acid is contacted with cellulosic material and held at a temperature in the range of preferably 160-220° C., and more preferably 165-195° C., for periods ranging from seconds to minutes to, e.g., 1 second to 60 minutes.

In another aspect, pretreatment is carried out as an ammonia fiber explosion step (AFEX pretreatment step).

In another aspect, pretreatment takes place in an aqueous slurry. In preferred aspects, cellulosic material is present during pretreatment in amounts preferably between 10-80 wt %, more preferably between 20-70 wt %, and most preferably between 30-60 wt %, such as around 50 wt %. The pretreated cellulosic material can be unwashed or washed using any method known in the art, e.g., washed with water.

Mechanical Pretreatment: The term “mechanical pretreatment” refers to various types of grinding or milling (e.g., dry milling, wet milling, or vibratory ball milling).

Physical Pretreatment: The term “physical pretreatment” refers to any pretreatment that promotes the separation and/or release of cellulose, hemicellulose, and/or lignin from cellulosic material. For example, physical pretreatment can involve irradiation (e.g., microwave irradiation), steaming/steam explosion, hydrothermolysis, and combinations thereof.

Physical pretreatment can involve high pressure and/or high temperature (steam explosion). In one aspect, high pressure means pressure in the range of preferably about 300 to about 600 psi, more preferably about 350 to about 550 psi, and most preferably about 400 to about 500 psi, such as around 450 psi. In another aspect, high temperature means temperatures in the range of about 100 to about 300° C., preferably about 140 to about 235° C. In a preferred aspect, mechanical pretreatment is performed in a batch-process, steam gun hydrolyzer system that uses high pressure and high temperature as defined above, e.g., a Sunds Hydrolyzer available from Sunds Defibrator AB, Sweden.

Combined Physical and Chemical Pretreatment: Cellulosic material can be pretreated both physically and chemically. For instance, the pretreatment step can involve dilute or mild acid treatment and high temperature and/or pressure treatment. The physical and chemical pretreatments can be carried out sequentially or simultaneously, as desired. A mechanical pretreatment can also be included.

Accordingly, in a preferred aspect, cellulosic material is subjected to mechanical, chemical, or physical pretreatment, or any combination thereof, to promote the separation and/or release of cellulose, hemicellulose, and/or lignin.

Biological Pretreatment: The term “biological pretreatment” refers to any biological pretreatment that promotes the separation and/or release of cellulose, hemicellulose, and/or lignin from cellulosic material. Biological pretreatment techniques can involve applying lignin-solubilizing microorganisms (see, for example, Hsu, T.-A., 1996, Pretreatment of biomass, in Handbook on Bioethanol: Production and Utilization, Wyman, C. E., ed., Taylor & Francis, Washington, D.C., 179-212; Ghosh and Singh, 1993, Physicochemical and biological treatments for enzymatic/microbial conversion of cellulosic biomass, Adv. Appl. Microbiol. 39: 295-333; McMillan, J. D., 1994, Pretreating lignocellulosic biomass: a review, in Enzymatic Conversion of Biomass for Fuels Production, Himmel, M. E., Baker, J. O., and Overend, R. P., eds., ACS Symposium Series 566, American Chemical Society, Washington, D.C., chapter 15; Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Olsson and Hahn-Hagerdal, 1996, Fermentation of lignocellulosic hydrolysates for ethanol production, Enz. Microb. Tech. 18: 312-331; and Vallander and Eriksson, 1990, Production of ethanol from lignocellulosic materials: State of the art, Adv. Biochem. Eng./Biotechnol. 42: 63-95).

Saccharification.

In the hydrolysis step, also known as saccharification, the cellulosic material, e.g., pretreated, is hydrolyzed to break down cellulose and alternatively also hemicellulose to fermentable sugars, such as glucose, cellobiose, xylose, xylulose, arabinose, mannose, galactose, and/or soluble oligosaccharides. The hydrolysis is performed enzymatically by an enzyme composition in the presence of a polypeptide having Berberine Bridge Enzyme Like activity of the present invention. The composition can further comprise one or more (several) hemicellulolytic or xylan degrading enzymes. The enzymes of the compositions can also be added sequentially.

Enzymatic hydrolysis is preferably carried out in a suitable aqueous environment under conditions that can be readily determined by one skilled in the art. In a preferred aspect, hydrolysis is performed under conditions suitable for the activity of the enzyme(s), i.e., optimal for the enzyme(s). The hydrolysis can be carried out as a fed batch or continuous process where the pretreated cellulosic material (substrate) is fed gradually to, for example, an enzyme containing hydrolysis solution.

The saccharification is generally performed in stirred-tank reactors or fermentors under controlled pH, temperature, and mixing conditions. Suitable process time, temperature and pH conditions can readily be determined by one skilled in the art. For example, the saccharification can last up to 200 hours, but is typically performed for preferably about 12 to about 96 hours, more preferably about 16 to about 72 hours, and most preferably about 24 to about 48 hours. The temperature is in the range of preferably about 25° C. to about 70° C., more preferably about 30° C. to about 65° C., and more preferably about 40° C. to 60° C., in particular about 50° C. The pH is in the range of preferably about 3 to about 8, more preferably about 3.5 to about 7, and most preferably about 4 to about 6, in particular about pH 5. The dry solids content is in the range of preferably about 5 to about 50 wt %, more preferably about 10 to about 40 wt %, and most preferably about 20 to about 30 wt %.

The enzyme composition preferably comprises enzymes having cellulolytic activity and/or xylan degrading activity. In one aspect, the enzyme composition comprises one or more (several) cellulolytic enzymes. In another aspect, the enzyme composition comprises one or more (several) xylan degrading enzymes. In another aspect, the enzyme composition comprises one or more (several) cellulolytic enzymes and one or more (several) xylan degrading enzymes.

The one or more (several) cellulolytic enzymes are preferably selected from the group consisting of an endoglucanase, a cellobiohydrolase, and a beta-glucosidase. The one or more (several) xylan degrading enzymes are preferably selected from the group consisting of a xylanase, an acetyxylan esterase, a feruloyl esterase, an arabinofuranosidase, a xylosidase, and a glucuronidase.

In another aspect, the enzyme composition further or even further comprises a polypeptide having cellulolytic enhancing activity (see, for example, WO 2005/074647, WO 2005/074656, and WO 2007/089290). In another aspect, the enzyme composition may further or even further comprise one or more (several) additional enzyme activities to improve the degradation of the cellulose-containing material. Preferred additional enzymes are hemicellulases (e.g., alpha-D-glucuronidases, alpha-L-arabinofuranosidases, endo-mannanases, beta-mannosidases, alpha-galactosidases, endo-alpha-L-arabinanases, beta-galactosidases), carbohydrate-esterases (e.g., acetyl-xylan esterases, acetyl-mannan esterases, ferulic acid esterases, coumaric acid esterases, glucuronoyl esterases), pectinases, proteases, ligninolytic enzymes (e.g., laccases, manganese peroxidases, lignin peroxidases, H₂O₂-producing enzymes, oxidoreductases), expansins, swollenins, or mixtures thereof. In the methods of the present invention, the additional enzyme(s) can be added prior to or during fermentation, e.g., during saccharification or during or after propagation of the fermenting microorganism(s).

One or more (several) components of the enzyme composition may be wild-type proteins, recombinant proteins, or a combination of wild-type proteins and recombinant proteins. For example, one or more (several) components may be native proteins of a cell, which is used as a host cell to express recombinantly one or more (several) other components of the enzyme composition. One or more (several) components of the enzyme composition may be produced as monocomponents, which are then combined to form the enzyme composition. The enzyme composition may be a combination of multicomponent and monocomponent protein preparations.

The enzymes used in the methods of the present invention may be in any form suitable for use in the processes described herein, such as, for example, a crude fermentation broth with or without cells removed, a cell lysate with or without cellular debris, a semi-purified or purified enzyme preparation, or a host cell as a source of the enzymes. The enzyme composition may be a dry powder or granulate, a non-dusting granulate, a liquid, a stabilized liquid, or a stabilized protected enzyme. Liquid enzyme preparations may, for instance, be stabilized by adding stabilizers such as a sugar, a sugar alcohol or another polyol, and/or lactic acid or another organic acid according to established processes.

The optimum amounts of the enzymes and polypeptides having Berberine Bridge Enzyme Like activity depend on several factors including, but not limited to, the mixture of component cellulolytic enzymes, the cellulosic substrate, the concentration of cellulosic substrate, the pretreatment(s) of the cellulosic substrate, temperature, time, pH, and inclusion of fermenting organism (e.g., yeast for Simultaneous Saccharification and Fermentation).

In a preferred aspect, an effective amount of cellulolytic enzyme(s) to cellulosic material is about 0.5 to about 50 mg, preferably at about 0.5 to about 40 mg, more preferably at about 0.5 to about 25 mg, more preferably at about 0.75 to about 20 mg, more preferably at about 0.75 to about 15 mg, even more preferably at about 0.5 to about 10 mg, and most preferably at about 2.5 to about 10 mg enzyme protein per g of cellulosic material.

In another preferred aspect, an effective amount of polypeptide(s) having Berberine Bridge Enzyme Like activity to cellulosic material is about 0.01 to about 50.0 mg, preferably about 0.01 to about 40 mg, more preferably about 0.01 to about 30 mg, more preferably about 0.01 to about 20 mg, more preferably about 0.01 to about 10 mg, more preferably about 0.01 to about 5 mg, more preferably at about 0.025 to about 1.5 mg, more preferably at about 0.05 to about 1.25 mg, more preferably at about 0.075 to about 1.25 mg, more preferably at about 0.1 to about 1.25 mg, even more preferably at about 0.15 to about 1.25 mg, and most preferably at about 0.25 to about 1.0 mg enzyme protein per g of cellulosic material.

In another preferred aspect, an effective amount of polypeptide(s) having Berberine Bridge Enzyme Like activity to cellulolytic enzyme(s) is about 0.005 to about 1.0 g, preferably at about 0.01 to about 1.0 g, more preferably at about 0.15 to about 0.75 g, more preferably at about 0.15 to about 0.5 g, more preferably at about 0.1 to about 0.5 g, even more preferably at about 0.1 to about 0.5 g, and most preferably at about 0.05 to about 0.2 g enzyme protein per g of cellulolytic enzyme(s).

The enzymes can be derived or obtained from any suitable origin, including, bacterial, fungal, yeast, plant, or mammalian origin. The term “obtained” means herein that the enzyme may have been isolated from an organism that naturally produces the enzyme as a native enzyme. The term “obtained” also means herein that the enzyme may have been produced recombinantly in a host organism employing methods described herein, wherein the recombinantly produced enzyme is either native or foreign to the host organism or has a modified amino acid sequence, e.g., having one or more (several) amino acids that are deleted, inserted and/or substituted, i.e., a recombinantly produced enzyme that is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained recombinantly, such as by site-directed mutagenesis or shuffling.

A polypeptide having cellulolytic enzyme activity or xylan degrading activity may be a bacterial polypeptide. For example, the polypeptide may be a gram positive bacterial polypeptide such as a Bacillus, Streptococcus, Streptomyces, Staphylococcus, Enterococcus, Lactobacillus, Lactococcus, Clostridium, Geobacillus, or Oceanobacillus polypeptide having cellulolytic enzyme activity or xylan degrading activity, or a Gram negative bacterial polypeptide such as an E. coli, Pseudomonas, Salmonella, Campylobacter, Helicobacter, Flavobacterium, Fusobacterium, Ilyobacter, Neisseria, or Ureaplasma polypeptide having cellulolytic enzyme activity or xylan degrading activity.

In a preferred aspect, the polypeptide is a Bacillus alkalophilus, Bacillus amyloliquefaciens, Bacillus brevis, Bacillus circulans, Bacillus clausii, Bacillus coagulans, Bacillus firmus, Bacillus lautus, Bacillus lentus, Bacillus licheniformis, Bacillus megaterium, Bacillus pumilus, Bacillus stearothermophilus, Bacillus subtilis, or Bacillus thuringiensis polypeptide having cellulolytic enzyme activity or xylan degrading activity.

In another preferred aspect, the polypeptide is a Streptococcus equisimilis, Streptococcus pyogenes, Streptococcus uberis, or Streptococcus equi subsp. Zooepidemicus polypeptide having cellulolytic enzyme activity or xylan degrading activity.

In another preferred aspect, the polypeptide is a Streptomyces achromogenes, Streptomyces avermitilis, Streptomyces coelicolor, Streptomyces griseus, or Streptomyces lividans polypeptide having cellulolytic enzyme activity or xylan degrading activity.

The polypeptide having cellulolytic enzyme activity or xylan degrading activity may also be a fungal polypeptide, and more preferably a yeast polypeptide such as a Candida, Kluyveromyces, Pichia, Saccharomyces, Schizosaccharomyces, or Yarrowia polypeptide having cellulolytic enzyme activity or xylan degrading activity; or more preferably a filamentous fungal polypeptide such as an Acremonium, Agaricus, Altemaria, Aspergillus, Aureobasidium, Botryosphaeria, Ceriporiopsis, Chaetomidium, Chrysosporium, Claviceps, Cochliobolus, Coprinopsis, Coptotermes, Corynascus, Ctyphonectria, Cryptococcus, Diplodia, Exidia, Filibasidium, Fusarium, Gibberella, Holomastigotoides, Humicola, Irpex, Lentinula, Leptospaeria, Magnaporthe, Melanocarpus, Meripilus, Mucor, Myceliophthora, Neocallimastix, Neurospora, Paecilomyces, Penicillium, Phanerochaete, Piromyces, Poitrasia, Pseudoplectania, Pseudotrichonympha, Rhizomucor, Schizophyllum, Scytalidium, Talaromyces, Thermoascus, Thielavia, Tolypocladium, Trichoderma, Trichophaea, Verticillium, Volvariella, or Xylaria polypeptide having cellulolytic enzyme activity or xylan degrading activity.

In a preferred aspect, the polypeptide is a Saccharomyces carlsbergensis, Saccharomyces cerevisiae, Saccharomyces diastaticus, Saccharomyces douglasii, Saccharomyces kluyveri, Saccharomyces norbensis, or Saccharomyces oviformis polypeptide having cellulolytic enzyme activity or xylan degrading activity.

In another preferred aspect, the polypeptide is an Acremonium cellulolyticus, Aspergillus aculeatus, Aspergillus awamori, Aspergillus fumigatus, Aspergillus foetidus, Aspergillus japonicus, Aspergillus nidulans, Aspergillus niger, Aspergillus oryzae, Chrysosporium keratinophilum, Chrysosporium lucknowense, Chrysosporium tropicum, Chrysosporium merdarium, Chrysosporium inops, Chrysosporium pannicola, Chrysosporium queenslandicum, Chrysosporium zonatum, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Humicola grisea, Humicola insolens, Humicola lanuginosa, Irpex lacteus, Mucor miehei, Myceliophthora thermophila, Neurospora crassa, Penicillium funiculosum, Penicillium purpurogenum, Phanerochaete chrysosporium, Thielavia achromatica, Thielavia albomyces, Thielavia albopilosa, Thielavia australeinsis, Thielavia fimeti, Thielavia microspora, Thielavia ovispora, Thielavia peruviana, Thielavia spededonium, Thielavia setosa, Thielavia subthermophila, Thielavia terrestris, Trichoderma harzianum, Trichoderma koningii, Trichoderma longibrachiatum, Trichoderma reesei, Trichoderma viride, or Trichophaea saccata polypeptide having cellulolytic enzyme activity or xylan degrading activity.

Chemically modified or protein engineered mutants of polypeptides having cellulolytic enzyme activity or xylan degrading activity may also be used.

One or more (several) components of the enzyme composition may be a recombinant component, i.e., produced by cloning of a DNA sequence encoding the single component and subsequent cell transformed with the DNA sequence and expressed in a host (see, for example, WO 91/17243 and WO 91/17244). The host is preferably a heterologous host (enzyme is foreign to host), but the host may under certain conditions also be a homologous host (enzyme is native to host). Monocomponent cellulolytic proteins may also be prepared by purifying such a protein from a fermentation broth.

Examples of commercial cellulolytic protein preparations suitable for use in the present invention include, for example, CELLIC™ Ctec (Novozymes A/S), CELLUCLAST™ (Novozymes A/S), NOVOZYM™ 188 (Novozymes A/S), CELLUZYME™ (Novozymes A/S), CEREFLO™ (Novozymes A/S), and ULTRAFLO™ (Novozymes A/S), ACCELERASE™ (Genencor Int.), LAMINEX™ (Genencor Int.), SPEZYME™ CP (Genencor Int.), ROHAMENT™ 7069 W (Röhm GmbH), FIBREZYME® LDI (Dyadic International, Inc.), FIBREZYME® LBR (Dyadic International, Inc.), or VISCOSTAR® 150 L (Dyadic International, Inc.). The cellulase enzymes are added in amounts effective from about 0.001 to about 5.0 wt % of solids, more preferably from about 0.025 to about 4.0 wt % of solids, and most preferably from about 0.005 to about 2.0 wt % of solids. The cellulase enzymes are added in amounts effective from about 0.001 to about 5.0 wt % of solids, more preferably from about 0.025 to about 4.0 wt % of solids, and most preferably from about 0.005 to about 2.0 wt % of solids.

Examples of bacterial endoglucanases that can be used in the methods of the present invention, include, but are not limited to, an Acidothermus cellulolyticus endoglucanase (WO 91/05039; WO 93/15186; U.S. Pat. No. 5,275,944; WO 96/02551; U.S. Pat. No. 5,536,655, WO 00/70031, WO 05/093050); Thermobifida fusca endoglucanase III (WO 05/093050); and Thermobifida fusca endoglucanase V (WO 05/093050).

Examples of fungal endoglucanases that can be used in the methods of the present invention, include, but are not limited to, a Trichoderma reesei endoglucanase I (Penttila et al., 1986, Gene 45: 253-263; GENBANK™ accession no. M15665); Trichoderma reesei endoglucanase II (Saloheimo, et al, 1988, Gene 63:11-22; GENBANK™ accession no. M19373); Trichoderma reesei endoglucanase III (Okada et al., 1988, Appl. Environ. Microbiol. 64: 555-563; GENBANK™ accession no. AB003694); and Trichoderma reesei endoglucanase V (Saloheimo et al., 1994, Molecular Microbiology 13: 219-228; GENBANK™ accession no. Z33381); Aspergillus aculeatus endoglucanase (Ooi et al., 1990, Nucleic Acids Research 18: 5884); Aspergillus kawachii endoglucanase (Sakamoto et al., 1995, Current Genetics 27: 435-439); Erwinia carotovara endoglucanase (Saarilahti et al., 1990, Gene 90: 9-14); Fusarium oxysporum endoglucanase (GENBANK™ accession no. L29381); Humicola grisea var. thermoidea endoglucanase (GENBANK™ accession no. AB003107); Melanocarpus albomyces endoglucanase (GENBANK™ accession no. MAL515703); Neurospora crassa endoglucanase (GENBANK™ accession no. XM_{—}324477); Humicola insolens endoglucanase V; Myceliophthora thermophila CBS 117.65 endoglucanase; basidiomycete CBS 495.95 endoglucanase; basidiomycete CBS 494.95 endoglucanase; Thielavia terrestris NRRL 8126 CEL6B endoglucanase; Thielavia terrestris NRRL 8126 CEL6C endoglucanase); Thielavia terrestris NRRL 8126 CEL7C endoglucanase; Thielavia terrestris NRRL 8126 CEL7E endoglucanase; Thielavia terrestris NRRL 8126 CEL7F endoglucanase; Cladorrhinum foecundissimum ATCC 62373 CEL7A endoglucanase; and Trichoderma reesei strain No. VTT-D-80133 endoglucanase (GENBANK™ accession no. M15665).

Examples of cellobiohydrolases useful in the methods of the present invention include, but are not limited to, Trichoderma reesei cellobiohydrolase I; Trichoderma reesei cellobiohydrolase II; Humicola insolens cellobiohydrolase I, Myceliophthora thermophila cellobiohydrolase II, Thielavia terrestris cellobiohydrolase II (CEL6A), Chaetomium thermophilum cellobiohydrolase I, and Chaetomium thermophilum cellobiohydrolase II.

Examples of beta-glucosidases useful in the methods of the present invention include, but are not limited to, Aspergillus oryzae beta-glucosidase; Aspergillus fumigatus beta-glucosidase; Penicillium brasilianum IBT 20888 beta-glucosidase; Aspergillus niger beta-glucosidase; and Aspergillus aculeatus beta-glucosidase.

The Aspergillus oryzae polypeptide having beta-glucosidase activity can be obtained according to WO 2002/095014. The Aspergillus fumigatus polypeptide having beta-glucosidase activity can be obtained according to WO 2005/047499. The Penicillium brasilianum polypeptide having beta-glucosidase activity can be obtained according to WO 2007/019442. The Aspergillus niger polypeptide having beta-glucosidase activity can be obtained according to Dan et al., 2000, J. Biol. Chem. 275: 4973-4980. The Aspergillus aculeatus polypeptide having beta-glucosidase activity can be obtained according to Kawaguchi et al., 1996, Gene 173: 287-288.

The beta-glucosidase may be a fusion protein. In one aspect, the beta-glucosidase is the Aspergillus oryzae beta-glucosidase variant BG fusion protein or the Aspergillus oryzae beta-glucosidase fusion protein obtained according to WO 2008/057637.

Other endoglucanases, cellobiohydrolases, and beta-glucosidases are disclosed in numerous Glycosyl Hydrolase families using the classification according to Henrissat B., 1991, A classification of glycosyl hydrolases based on amino-acid sequence similarities, Biochem. J. 280: 309-316, and Henrissat B., and Bairoch A., 1996, Updating the sequence-based classification of glycosyl hydrolases, Biochem. J. 316: 695-696.

Other cellulolytic enzymes that may be used in the present invention are described in EP 495,257, EP 531,315, EP 531,372, WO 89/09259, WO 94/07998, WO 95/24471, WO 96/11262, WO 96/29397, WO 96/034108, WO 97/14804, WO 98/08940, WO 98/012307, WO 98/13465, WO 98/015619, WO 98/015633, WO 98/028411, WO 99/06574, WO 99/10481, WO 99/025846, WO 99/025847, WO 99/031255, WO 2000/009707, WO 2002/050245, WO 2002/0076792, WO 2002/101078, WO 2003/027306, WO 2003/052054, WO 2003/052055, WO 2003/052056, WO 2003/052057, WO 2003/052118, WO 2004/016760, WO 2004/043980, WO 2004/048592, WO 2005/001065, WO 2005/028636, WO 2005/093050, WO 2005/093073, WO 2006/074005, WO 2006/117432, WO 2007/071818, WO 2007/071820, WO 2008/008070, WO 2008/008793, U.S. Pat. No. 4,435,307, U.S. Pat. No. 5,457,046, U.S. Pat. No. 5,648,263, U.S. Pat. No. 5,686,593, U.S. Pat. No. 5,691,178, U.S. Pat. No. 5,763,254, and U.S. Pat. No. 5,776,757.

In the methods of the present invention, any GH61 polypeptide having cellulolytic enhancing activity can be used.

In a first aspect, the polypeptide having cellulolytic enhancing activity comprises the following motifs:

[ILMV]-P-X(4,5)-G-X-Y-[ILMV]-X-R-X-[EQ]-X(4)-[HNQ]
and
[FW]-[TF]-K-[AIV],

wherein X is any amino acid, X(4,5) is any amino acid at 4 or 5 contiguous positions, and X(4) is any amino acid at 4 contiguous positions.

The polypeptide comprising the above-noted motifs may further comprise:

H-X(1,2)-G-P-X(3)-[YW]-[AILMV],
[EQ]-X-Y-X(2)-C-X-[EHQN]-[FILV]-X-[ILV],
or
H-X(1,2)-G-P-X(3)-[YW]-[AILMV] and [EQ]-X-Y-X(2)-
C-X-[EHQN]-[FILV]-X-[ILV],

wherein X is any amino acid, X(1,2) is any amino acid at 1 position or 2 contiguous positions, X(3) is any amino acid at 3 contiguous positions, and X(2) is any amino acid at 2 contiguous positions. In the above motifs, the accepted IUPAC single letter amino acid abbreviation is employed.

In a preferred aspect, the polypeptide having cellulolytic enhancing activity further comprises H-X(1,2)-G-P-X(3)-[YW]-[AILMV]. In another preferred aspect, the isolated polypeptide having cellulolytic enhancing activity further comprises [EQ]-X-Y-X(2)-C-X-[EHQN]-[FILV]-X-[ILV]. In another preferred aspect, the polypeptide having cellulolytic enhancing activity further comprises H-X(1,2)-G-P-X(3)-[YW]-[AILMV] and [EQ]-X-Y-X(2)-C-X-[EHQN]-[FILV]-X-[ILV].

In a second aspect, the polypeptide having cellulolytic enhancing activity comprises the following motif:

[ILMV]-P-x(4,5)-G-x-Y-[ILMV]-x-R-x-[EQ]-x(3)-A-
[HNQ],

wherein x is any amino acid, x(4,5) is any amino acid at 4 or 5 contiguous positions, and x(3) is any amino acid at 3 contiguous positions. In the above motif, the accepted IUPAC single letter amino acid abbreviation is employed.

Examples of polypeptides having cellulolytic enhancing activity useful in the methods of the present invention include, but are not limited to, polypeptides having cellulolytic enhancing activity from Thielavia terrestris (WO 2005/074647); polypeptides having cellulolytic enhancing activity from Thermoascus aurantiacus (WO 2005/074656); polypeptides having cellulolytic enhancing activity from Trichoderma reesei (WO 2007/089290); and polypeptides having cellulolytic enhancing activity from Myceliophthora thermophila (WO 2009/085935; WO 2009/085859; WO 2009/085864; WO 2009/085868).

Examples of commercial xylan degrading enzyme preparations suitable for use in the present invention include, for example, SHEARZYME™ (Novozymes A/S), CELLIC™ Htec (Novozymes A/S), VISCOZYME® (Novozymes A/S), ULTRAFLO® (Novozymes A/S), PULPZYME® HC (Novozymes A/S), MULTIFECT® Xylanase (Genencor), ECOPULP® TX-200A (AB Enzymes), HSP 6000 Xylanase (DSM), DEPOL™ 333 P (Biocatalysts Limit, Wales, UK), DEPOL™ 740 L. (Biocatalysts Limit, Wales, UK), and DEPOL™ 762 P (Biocatalysts Limit, Wales, UK).

Examples of xylanases useful in the methods of the present invention include, but are not limited to, Aspergillus aculeatus xylanase (GeneSeqP:AAR63790; WO 94/21785), Aspergillus fumigatus xylanases (WO 2006/078256), and Thielavia terrestris NRRL 8126 xylanases (WO 2009/079210).

Examples of beta-xylosidases useful in the methods of the present invention include, but are not limited to, Trichoderma reesei beta-xylosidase (UniProtKB/TrEMBL accession number Q92458), Talaromyces emersonii (SwissProt accession number Q8X212), and Neurospora crassa (SwissProt accession number Q7SOW4).

Examples of acetylxylan esterases useful in the methods of the present invention include, but are not limited to, Hypocrea jecorina acetylxylan esterase (WO 2005/001036), Neurospora crassa acetylxylan esterase (UniProt accession number q7s259), Thielavia terrestris NRRL 8126 acetylxylan esterase (WO 2009/042846), Chaetomium globosum acetylxylan esterase (Uniprot accession number Q2GWX4), Chaetomium gracile acetylxylan esterase (GeneSeqP accession number AAB82124), Phaeosphaeria nodorum acetylxylan esterase (Uniprot accession number Q0UHJ1), and Humicola insolens DSM 1800 acetylxylan esterase (WO 2009/073709).

Examples of ferulic acid esterases useful in the methods of the present invention include, but are not limited to, Humicola insolens DSM 1800 feruloyl esterase (WO 2009/076122), Neurospora crassa feruloyl esterase (UniProt accession number Q9HGR3), and Neosartorya fischeri feruloyl esterase (UniProt Accession number A1D9T4).

Examples of arabinofuranosidases useful in the methods of the present invention include, but are not limited to, Humicola insolens DSM 1800 arabinofuranosidase (WO 2009/073383) and Aspergillus niger arabinofuranosidase (GeneSeqP accession number AAR94170).

Examples of alpha-glucuronidases useful in the methods of the present invention include, but are not limited to, Aspergillus clavatus alpha-glucuronidase (UniProt accession number alcc12), Trichoderma reesei alpha-glucuronidase (Uniprot accession number Q99024), Talaromyces emersonii alpha-glucuronidase (UniProt accession number Q8X211), Aspergillus niger alpha-glucuronidase (Uniprot accession number Q96WX9), Aspergillus terreus alpha-glucuronidase (SwissProt accession number Q0CJP9), and Aspergillus fumigatus alpha-glucuronidase (SwissProt accession number Q4WW45).

The enzymes and proteins used in the methods of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, CA, 1991). Suitable media are available from commercial suppliers or may be prepared according to published compositions (e.g., in catalogues of the American Type Culture Collection). Temperature ranges and other conditions suitable for growth and enzyme production are known in the art (see, e.g., Bailey, J. E., and Ollis, D. F., Biochemical Engineering Fundamentals, McGraw-Hill Book Company, NY, 1986).

The fermentation can be any method of cultivation of a cell resulting in the expression or isolation of an enzyme. Fermentation may, therefore, be understood as comprising shake flask cultivation, or small- or large-scale fermentation (including continuous, batch, fed-batch, or solid state fermentations) in laboratory or industrial fermentors performed in a suitable medium and under conditions allowing the enzyme to be expressed or isolated. The resulting enzymes produced by the methods described above may be recovered from the fermentation medium and purified by conventional procedures.

Fermentation.

The fermentable sugars obtained from the hydrolyzed cellulosic material can be fermented by one or more (several) fermenting microorganisms capable of fermenting the sugars directly or indirectly into a desired fermentation product. “Fermentation” or “fermentation process” refers to any fermentation process or any process comprising a fermentation step. Fermentation processes also include fermentation processes used in the consumable alcohol industry (e.g., beer and wine), dairy industry (e.g., fermented dairy products), leather industry, and tobacco industry. The fermentation conditions depend on the desired fermentation product and fermenting organism and can easily be determined by one skilled in the art.

In the fermentation step, sugars, released from cellulosic material as a result of the pretreatment and enzymatic hydrolysis steps, are fermented to a product, e.g., ethanol, by a fermenting organism, such as yeast. Hydrolysis (saccharification) and fermentation can be separate or simultaneous, as described herein.

Any suitable hydrolyzed cellulosic material can be used in the fermentation step in practicing the present invention. The material is generally selected based on the desired fermentation product, i.e., the substance to be obtained from the fermentation, and the process employed, as is well known in the art.

The term “fermentation medium” is understood herein to refer to a medium before the fermenting microorganism(s) is(are) added, such as, a medium resulting from a saccharification process, as well as a medium used in a simultaneous saccharification and fermentation process (SSF).

“Fermenting microorganism” refers to any microorganism, including bacterial and fungal organisms, suitable for use in a desired fermentation process to produce a fermentation product. The fermenting organism can be C₆ and/or C₅ fermenting organisms, or a combination thereof. Both C₆ and C₅ fermenting organisms are well known in the art. Suitable fermenting microorganisms are able to ferment, i.e., convert, sugars, such as glucose, xylose, xylulose, arabinose, maltose, mannose, galactose, or oligosaccharides, directly or indirectly into the desired fermentation product.

Examples of bacterial and fungal fermenting organisms producing ethanol are described by Lin et al, 2006, Appl. Microbiol. Biotechnol. 69: 627-642.

Examples of fermenting microorganisms that can ferment C₆ sugars include bacterial and fungal organisms, such as yeast. Preferred yeast includes strains of the Saccharomyces spp., preferably Saccharomyces cerevisiae.

Examples of fermenting organisms that can ferment C₅ sugars include bacterial and fungal organisms, such as yeast. Preferred C₅ fermenting yeast include strains of Pichia, preferably Pichia stipitis, such as Pichia stipitis CBS 5773; strains of Candida, preferably Candida boidinii, Candida brassicae, Candida sheatae, Candida diddensii, Candida pseudotropicalis, or Candida utilis.

Other fermenting organisms include strains of Zymomonas, such as Zymomonas mobilis; Hansenula, such as Hansenula anomala; Kluyveromyces, such as K. fragilis; Schizosaccharomyces, such as S. pombe; and E. coli, especially E. coli strains that have been genetically modified to improve the yield of ethanol.

In a preferred aspect, the yeast is a Saccharomyces spp. In a more preferred aspect, the yeast is Saccharomyces cerevisiae. In another more preferred aspect, the yeast is Saccharomyces distaticus. In another more preferred aspect, the yeast is Saccharomyces uvarum. In another preferred aspect, the yeast is a Kluyveromyces. In another more preferred aspect, the yeast is Kluyveromyces marxianus. In another more preferred aspect, the yeast is Kluyveromyces fragilis. In another preferred aspect, the yeast is a Candida. In another more preferred aspect, the yeast is Candida boidinii. In another more preferred aspect, the yeast is Candida brassicae. In another more preferred aspect, the yeast is Candida diddensii. In another more preferred aspect, the yeast is Candida pseudotropicalis. In another more preferred aspect, the yeast is Candida utilis. In another preferred aspect, the yeast is a Clavispora. In another more preferred aspect, the yeast is Clavispora lusitaniae. In another more preferred aspect, the yeast is Clavispora opuntiae. In another preferred aspect, the yeast is a Pachysolen. In another more preferred aspect, the yeast is Pachysolen tannophilus. In another preferred aspect, the yeast is a Pichia. In another more preferred aspect, the yeast is a Pichia stipitis. In another preferred aspect, the yeast is a Bretannomyces. In another more preferred aspect, the yeast is Bretannomyces clausenii (Philippidis, G. P., 1996, Cellulose bioconversion technology, in Handbook on Bioethanol: Production and Utilization, Wyman, C. E., ed., Taylor & Francis, Washington, D.C., 179-212).

Bacteria that can efficiently ferment hexose and pentose to ethanol include, for example, Zymomonas mobilis and Clostridium thermocellum (Philippidis, 1996, supra).

In a preferred aspect, the bacterium is a Zymomonas. In a more preferred aspect, the bacterium is Zymomonas mobilis. In another preferred aspect, the bacterium is a Clostridium. In another more preferred aspect, the bacterium is Clostridium thermocellum.

Commercially available yeast suitable for ethanol production includes, e.g., ETHANOL RED™ yeast (available from Fermentis/Lesaffre, USA), FALI™ (available from Fleischmann's Yeast, USA), SUPERSTART™ and THERMOSACC™ fresh yeast (available from Ethanol Technology, WI, USA), BIOFERM™ AFT and XR (available from NABC—North American Bioproducts Corporation, GA, USA), GERT STRAND™ (available from Gert Strand AB, Sweden), and FERMIOL™ (available from DSM Specialties).

In a preferred aspect, the fermenting microorganism has been genetically modified to provide the ability to ferment pentose sugars, such as xylose utilizing, arabinose utilizing, and xylose and arabinose co-utilizing microorganisms.

The cloning of heterologous genes into various fermenting microorganisms has led to the construction of organisms capable of converting hexoses and pentoses to ethanol (cofermentation) (Chen and Ho, 1993, Cloning and improving the expression of Pichia stipitis xylose reductase gene in Saccharomyces cerevisiae, Appl. Biochem. Biotechnol. 39-40: 135-147; Ho et al., 1998, Genetically engineered Saccharomyces yeast capable of effectively cofermenting glucose and xylose, Appl. Environ. Microbiol. 64: 1852-1859; Kotter and Ciriacy, 1993, Xylose fermentation by Saccharomyces cerevisiae, Appl. Microbiol. Biotechnol. 38: 776-783; Walfridsson et al., 1995, Xylose-metabolizing Saccharomyces cerevisiae strains overexpressing the TKL1 and TAL1 genes encoding the pentose phosphate pathway enzymes transketolase and transaldolase, Appl. Environ. Microbiol 61: 4184-4190; Kuyper et al., 2004, Minimal metabolic engineering of Saccharomyces cerevisiae for efficient anaerobic xylose fermentation: a proof of principle, FEMS Yeast Research 4: 655-664; Beall et al, 1991, Parametric studies of ethanol production from xylose and other sugars by recombinant Escherichia coli, Biotech. Bioeng. 38: 296-303; Ingram et al., 1998, Metabolic engineering of bacteria for ethanol production, Biotechnol. Bioeng. 58: 204-214; Zhang et al., 1995, Metabolic engineering of a pentose metabolism pathway in ethanologenic Zymomonas mobilis, Science 267: 240-243; Deanda et al, 1996, Development of an arabinose-fermenting Zymomonas mobilis strain by metabolic pathway engineering, Appl. Environ. Microbiol. 62: 4465-4470; WO 2003/062430, xylose isomerase).

In a preferred aspect, the genetically modified fermenting microorganism is Saccharomyces cerevisiae. In another preferred aspect, the genetically modified fermenting microorganism is Zymomonas mobilis. In another preferred aspect, the genetically modified fermenting microorganism is Escherichia coli. In another preferred aspect, the genetically modified fermenting microorganism is Klebsiella oxytoca. In another preferred aspect, the genetically modified fermenting microorganism is Kluyveromyces sp.

It is well known in the art that the organisms described above can also be used to produce other substances, as described herein.

The fermenting microorganism is typically added to the degraded lignocellulose or hydrolysate and the fermentation is performed for about 8 to about 96 hours, such as about 24 to about 60 hours. The temperature is typically between about 26° C. to about 60° C., in particular about 32° C. or 50° C., and at about pH 3 to about pH 8, such as around pH 4-5, 6, or 7.

In a preferred aspect, the yeast and/or another microorganism is applied to the degraded cellulosic material and the fermentation is performed for about 12 to about 96 hours, such as typically 24-60 hours. In a preferred aspect, the temperature is preferably between about 20° C. to about 60° C., more preferably about 25° C. to about 50° C., and most preferably about 32° C. to about 50° C., in particular about 32° C. or 50° C., and the pH is generally from about pH 3 to about pH 7, preferably around pH 4-7. However, some fermenting organisms, e.g., bacteria, have higher fermentation temperature optima. Yeast or another microorganism is preferably applied in amounts of approximately 10⁵ to 10¹², preferably from approximately 10⁷ to 10¹⁰, especially approximately 2×10⁸ viable cell count per ml of fermentation broth. Further guidance in respect of using yeast for fermentation can be found in, e.g., “The Alcohol Textbook” (Editors K. Jacques, T. P. Lyons and D. R. Kelsall, Nottingham University Press, United Kingdom 1999), which is hereby incorporated by reference.

For ethanol production, following the fermentation the fermented slurry is distilled to extract the ethanol. The ethanol obtained according to the methods of the invention can be used as, e.g., fuel ethanol, drinking ethanol, i.e., potable neutral spirits, or industrial ethanol.

A fermentation stimulator can be used in combination with any of the processes described herein to further improve the fermentation process, and in particular, the performance of the fermenting microorganism, such as, rate enhancement and ethanol yield. A “fermentation stimulator” refers to stimulators for growth of the fermenting microorganisms, in particular, yeast. Preferred fermentation stimulators for growth include vitamins and minerals. Examples of vitamins include multivitamins, biotin, pantothenate, nicotinic acid, meso-inositol, thiamine, pyridoxine, para-aminobenzoic acid, folic acid, riboflavin, and Vitamins A, B, C, D, and E. See, for example, Alfenore et al., Improving ethanol production and viability of Saccharomyces cerevisiae by a vitamin feeding strategy during fed-batch process, Springer-Verlag (2002), which is hereby incorporated by reference. Examples of minerals include minerals and mineral salts that can supply nutrients comprising P, K, Mg, S, Ca, Fe, Zn, Mn, and Cu.

Fermentation Products:

A fermentation product can be any substance derived from the fermentation. The fermentation product can be, without limitation, an alcohol (e.g., arabinitol, butanol, ethanol, glycerol, methanol, 1,3-propanediol, sorbitol, and xylitol); an organic acid (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, oxaloacetic acid, propionic acid, succinic acid, and xylonic acid); a ketone (e.g., acetone); an amino acid (e.g., aspartic acid, glutamic acid, glycine, lysine, serine, and threonine); and a gas (e.g., methane, hydrogen (H₂), carbon dioxide (CO₂), and carbon monoxide (CO)). The fermentation product can also be protein as a high value product.

In a preferred aspect, the fermentation product is an alcohol. It will be understood that the term “alcohol” encompasses a substance that contains one or more hydroxyl moieties. In a more preferred aspect, the alcohol is arabinitol. In another more preferred aspect, the alcohol is butanol. In another more preferred aspect, the alcohol is ethanol. In another more preferred aspect, the alcohol is glycerol. In another more preferred aspect, the alcohol is methanol. In another more preferred aspect, the alcohol is 1,3-propanediol. In another more preferred aspect, the alcohol is sorbitol. In another more preferred aspect, the alcohol is xylitol. See, for example, Gong, C. S., Cao, N. J., Du, J., and Tsao, G. T., 1999, Ethanol production from renewable resources, in Advances in Biochemical Engineering/Biotechnology, Scheper, T., ed., Springer-Verlag Berlin Heidelberg, Germany, 65: 207-241; Silveira, M. M., and Jonas, R., 2002, The biotechnological production of sorbitol, Appl. Microbiol. Biotechnol. 59: 400-408; Nigam, P., and Singh, D., 1995, Processes for fermentative production of xylitol—a sugar substitute, Process Biochemistry 30 (2): 117-124; Ezeji, T. C., Qureshi, N. and Blaschek, H. P., 2003, Production of acetone, butanol and ethanol by Clostridium beijerinckii BA101 and in situ recovery by gas stripping, World Journal of Microbiology and Biotechnology 19 (6): 595-603.

In another preferred aspect, the fermentation product is an organic acid. In another more preferred aspect, the organic acid is acetic acid. In another more preferred aspect, the organic acid is acetonic acid. In another more preferred aspect, the organic acid is adipic acid. In another more preferred aspect, the organic acid is ascorbic acid. In another more preferred aspect, the organic acid is citric acid. In another more preferred aspect, the organic acid is 2,5-diketo-D-gluconic acid. In another more preferred aspect, the organic acid is formic acid. In another more preferred aspect, the organic acid is fumaric acid. In another more preferred aspect, the organic acid is glucaric acid. In another more preferred aspect, the organic acid is gluconic acid. In another more preferred aspect, the organic acid is glucuronic acid. In another more preferred aspect, the organic acid is glutaric acid. In another preferred aspect, the organic acid is 3-hydroxypropionic acid. In another more preferred aspect, the organic acid is itaconic acid. In another more preferred aspect, the organic acid is lactic acid. In another more preferred aspect, the organic acid is malic acid. In another more preferred aspect, the organic acid is malonic acid. In another more preferred aspect, the organic acid is oxalic acid. In another more preferred aspect, the organic acid is propionic acid. In another more preferred aspect, the organic acid is succinic acid. In another more preferred aspect, the organic acid is xylonic acid. See, for example, Chen, R., and Lee, Y. Y., 1997, Membrane-mediated extractive fermentation for lactic acid production from cellulosic biomass, Appl. Biochem. Biotechnol. 63-65: 435-448.

In another preferred aspect, the fermentation product is a ketone. It will be understood that the term “ketone” encompasses a substance that contains one or more ketone moieties. In another more preferred aspect, the ketone is acetone. See, for example, Qureshi and Blaschek, 2003, supra.

In another preferred aspect, the fermentation product is an amino acid. In another more preferred aspect, the organic acid is aspartic acid. In another more preferred aspect, the amino acid is glutamic acid. In another more preferred aspect, the amino acid is glycine. In another more preferred aspect, the amino acid is lysine. In another more preferred aspect, the amino acid is serine. In another more preferred aspect, the amino acid is threonine. See, for example, Richard, A., and Margaritis, A., 2004, Empirical modeling of batch fermentation kinetics for poly(glutamic acid) production and other microbial biopolymers, Biotechnology and Bioengineering 87 (4): 501-515.

In another preferred aspect, the fermentation product is a gas. In another more preferred aspect, the gas is methane. In another more preferred aspect, the gas is H₂. In another more preferred aspect, the gas is CO₂. In another more preferred aspect, the gas is CO. See, for example, Kataoka, N., A. Miya, and K. Kiriyama, 1997, Studies on hydrogen production by continuous culture system of hydrogen-producing anaerobic bacteria, Water Science and Technology 36 (6-7): 41-47; and Gunaseelan V. N. in Biomass and Bioenergy, Vol. 13 (1-2), pp. 83-114, 1997, Anaerobic digestion of biomass for methane production: A review.

Recovery.

The fermentation product(s) can be optionally recovered from the fermentation medium using any method known in the art including, but not limited to, chromatography, electrophoretic procedures, differential solubility, distillation, or extraction. For example, alcohol is separated from the fermented cellulosic material and purified by conventional methods of distillation. Ethanol with a purity of up to about 96 vol. % can be obtained, which can be used as, for example, fuel ethanol, drinking ethanol, i.e., potable neutral spirits, or industrial ethanol.

### Signal Peptide and Propeptide

The present invention also relates to an isolated polynucleotide encoding a signal peptide comprising or consisting of amino acids 1 to 20 of SEQ ID NO: 4, amino acids 1 to 20 of SEQ ID NO: 6, amino acids 1 to 13 of SEQ ID NO: 8, amino acids 1 to 20 of SEQ ID NO: 10, amino acids 1 to 19 of SEQ ID NO: 12, amino acids 1 to 18 of SEQ ID NO: 14 or amino acids 1 to 20 of SEQ ID NO: 16. The present invention also relates to an isolated polynucleotide encoding a propeptide comprising or consisting of amino acids 1 to 20 of SEQ ID NO: 4, amino acids 1 to 20 of SEQ ID NO: 6, amino acids 1 to 13 of SEQ ID NO: 8, amino acids 1 to 20 of SEQ ID NO: 10, amino acids 1 to 19 of SEQ ID NO: 12, amino acids 1 to 18 of SEQ ID NO: 14 or amino acids 1 to 20 of SEQ ID NO: 16. The present invention also relates to an isolated polynucleotide encoding a signal peptide and a propeptide comprising or consisting of amino acids 1 to 20 of SEQ ID NO: 4, amino acids 1 to 20 of SEQ ID NO: 6, amino acids 1 to 13 of SEQ ID NO: 8, amino acids 1 to 20 of SEQ ID NO: 10, amino acids 1 to 19 of SEQ ID NO: 12, amino acids 1 to 18 of SEQ ID NO: 14 or amino acids 1 to 20 of SEQ ID NO: 16. The polynucleotides may further comprise a gene encoding a protein, which is operably linked to the signal peptide and/or propeptide. The protein is preferably foreign to the signal peptide and/or propeptide. In one aspect, the polynucleotide encoding the signal peptide is nucleotides 1 to 60 of SEQ ID NO: 3, 1 to 60 of SEQ ID NO: 5, 1 to 39 of SEQ ID NO: 7, 1 to 60 of SEQ ID NO: 9, 1 to 57 of SEQ ID NO: 11, 1 to 54 of SEQ ID NO: 13 or 1 to 60 of SEQ ID NO: 15.

The present invention also relates to nucleic acid constructs, expression vectors and recombinant host cells comprising such polynucleotides.

The present invention also relates to methods of producing a protein, comprising (a) cultivating a recombinant host cell comprising such polynucleotide; and (b) recovering the protein.

The protein may be native or heterologous to a host cell. The term “protein” is not meant herein to refer to a specific length of the encoded product and, therefore, encompasses peptides, oligopeptides, and polypeptides. The term “protein” also encompasses two or more polypeptides combined to form the encoded product. The proteins also include hybrid polypeptides and fused polypeptides.

Preferably, the protein is a hormone, enzyme, receptor or portion thereof, antibody or portion thereof, or reporter. For example, the protein may be a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, or beta-xylosidase.

The gene may be obtained from any prokaryotic, eukaryotic, or other source.

The present invention is further described by the following examples that should not be construed as limiting the scope of the invention.

### EXAMPLES

### Materials

Chemicals used as buffers and substrates were commercial products of at least reagent grade.

### Strains

Aspergillus aculeatus CBS 172.66 and Penicillium pinophilum was used as the source of polypeptides having BBEL enzymatic activity. Aspergillus oryzae MT3568 strain was used for expression of the BBEL genes encoding the polypeptides having BBEL enzymatic activity. A. oryzae MT3568 is an amdS (acetamidase) disrupted gene derivative of Aspergillus oryzae JaL355 (WO 2002/40694) in which pyrG auxotrophy was restored by disrupting the A. oryzae acetamidase (amdS) gene with the pyrG gene.

### Media and Solutions

YP+2% glucose medium was composed of 1% yeast extract, 2% peptone and 2% glucose.

YP+2% maltodextrin medium was composed of 1% yeast extract, 2% peptone and 2% maltodextrin.

PDA agar plates were composed of potato infusion (potato infusion was made by boiling 300 g of sliced (washed but unpeeled) potatoes in water for 30 minutes and then decanting or straining the broth through cheesecloth. Distilled water was then added until the total volume of the suspension was one liter, followed by 20 g of dextrose and 20 g of agar powder. The medium was sterilized by autoclaving at 15 psi for 15 minutes (Bacteriological Analytical Manual, 8th Edition, Revision A, 1998).

LB plates were composed of 10 g of Bacto-Tryptone, 5 g of yeast extract, 10 g of sodium chloride, 15 g of Bacto-agar, and deionized water to 1 liter.

LB medium was composed of 10 g of Bacto-Tryptone, 5 g of yeast extract, 10 g of sodium chloride, and deionized water to 1 liter.

COVE sucrose plates were composed of 342 g of sucrose, 20 g of agar powder, 20 ml of COVE salts solution, and deionized water to 1 liter. The medium was sterilized by autoclaving at 15 psi for 15 minutes (Bacteriological Analytical Manual, 8th Edition, Revision A, 1998). The medium was cooled to 60° C. and 10 mM acetamide, 15 mM CsCl, TRITON® X-100 (50 μl/500 ml) were added.

COVE salts solution was composed of 26 g of MgSO₄.7H₂O, 26 g of KCL, 26 g of KH₂PO₄, 50 ml of COVE trace metals solution, and deionized water to 1 liter.

COVE trace metals solution was composed of 0.04 g of Na₂B₄O₇.10H₂O, 0.4 g of CuSO₄.5H₂O, 1.2 g of FeSO₄.7H₂O, 0.7 g of MnSO₄—H₂O, 0.8 g of Na₂MoO₄.2H₂O, 10 g of ZnSO₄.7H₂O, and deionized water to 1 liter.

### Example 1

### Source of DNA Sequence Information for Aspergillus aculeatus CBS 172.66 and Penicillium pinophilum

Genomic sequence information was generated by the U.S. Department of Energy Joint Genome Institute (JGI). A preliminary assembly of the genome was downloaded from JGI and analyzed using the Pedant-Pro™ Sequence Analysis Suite (Biomax Informatics AG, Martinsried, Germany). Gene models constructed by the software were used as a starting point for detecting BBEL homologues in the genome. More precise gene models were constructed manually using multiple known BBEL protein sequences as a guide.

### Example 2

### Aspergillus aculeatus CBS 172.66 and Penicillium pinophilum Genomic DNA Extraction

Aspergillus aculeatus CBS 172.66 and Penicillium pinophilum was propagated on PDA agar plates at 26° C. for 7 days. Spores harvested from the PDA plates were used to inoculate 25 ml of YP+2% glucose medium in a baffled shake flask and incubated at 26° C. for 48 hours with agitation at 200 rpm.

Genomic DNA was isolated according to a modified FASTDNA® SPIN protocol (Qbiogene, Inc., Carlsbad, Calif., USA). Briefly a FASTDNA® SPIN Kit for Soil (Qbiogene, Inc., Carlsbad, Calif., USA) was used in a FASTPREP® 24 Homogenization System (MP Biosciences, Santa Ana, Calif., USA). Two ml of fungal material from the above culture was harvested by centrifugation at 14,000×g for 2 minutes. The supernatant was removed and the pellet resuspended in 500 μl of deionized water. The suspension was transferred to a Lysing Matrix E FASTPREP® tube (Qbiogene, Inc., Carlsbad, Calif., USA) and 790 μl of sodium phosphate buffer and 100 μl of MT buffer from the FASTDNA® SPIN Kit were added to the tube. The sample was then secured in the FASTPREP® Instrument (Qbiogene, Inc., Carlsbad, Calif., USA) and processed for 60 seconds at a speed of 5.5 m/sec. The sample was then centrifuged at 14,000×g for two minutes and the supernatant transferred to a clean EPPENDORF® tube. A 250 μl volume of PPS reagent from the FASTDNA® SPIN Kit was added and then the sample was mixed gently by inversion. The sample was again centrifuged at 14,000×g for 5 minutes. The supernatant was transferred to a 15 ml tube followed by 1 ml of Binding Matrix suspension from the FASTDNA® SPIN Kit and then mixed by inversion for two minutes. The sample was placed in a stationary tube rack and the silica matrix was allowed to settle for 3 minutes. A 500 μl volume of the supernatant was removed and discarded and then the remaining sample was resuspended in the matrix. The sample was then transferred to a SPIN filter tube from the FASTDNA® SPIN Kit and centrifuged at 14,000×g for 1 minute. The catch tube was emptied and the remaining matrix suspension added to the SPIN filter tube. The sample was again centrifuged at 14,000×g for 1 minute. A 500 μl volume of SEWS-M solution from the FASTDNA® SPIN Kit was added to the SPIN filter tube and the sample was centrifuged at the same speed for 1 minute. The catch tube was emptied and the SPIN filter replaced in the catch tube. The unit was centrifuged at 14,000×g for 2 minutes to dry the matrix of residual SEWS-M wash solution. The SPIN filter was placed in a fresh catch tube and allowed to air dry for 5 minutes at room temperature. The matrix was gently resuspended in 100 μl of DES (DNase/Pyrogen free water) with a pipette tip. The unit was centrifuged at 14,000×g for 1 minute to elute the genomic DNA followed by elution with 100 μl of 0.1 mM EDTA-10 mM Tris pH 8.0 by centrifugation at 14,000×g for 1 minute and the eluates were combined. The concentration of the DNA harvested from the catch tube was measured at 260 nm with a UV spectrophotometer.

### Example 3

### Construction of an Aspergillus oryzae Expression Vector Containing Genomic Sequence Encoding a BBEL Polypeptide Having Cellulolytic Enhancing Activity

Two synthetic oligonucleotide primers shown below were designed to amplify by PCR the Aspergillus aculeatus CBS 172.66 P244GP BBEL gene from the genomic DNA prepared in Example 2. An IN-FUSION® Cloning Kit (Clontech Laboratories, Inc., Mountain View, Calif., USA) was used to clone the fragment directly into the expression vector pDau109 (WO 2005/042735).

Primer F-P244GP:
5′-ACACAACTGGGGATCCACCATGTATCGGTTCAAGCTGGAATT-3′
Primer R-P244GP:
5′-AGATCTCGAGAAGCTTATCACGCAACAGGCGCATAAA-3′

Bold letters represent coding sequence. The underlined sequence is homologous to insertion sites of pDau109. The PCR reaction (25 μl) was composed of 12.5 μl of 2×IPROOF™ HF Master Mix, 0.5 μl of primer F-P244GP (100 μM), 0.5 μl of primer R-P244GP (100 μM), 0.5 μl of genomic (100 ng/μl), and 11 μl of deionized water. The PCR reaction was incubated in a DYAD® Dual-Block Thermal Cycler (MJ Research Inc., Waltham, Mass., USA) programmed for 1 cycle at 98° C. for 30 seconds; 30 cycles each at 98° C. for 10 seconds, 55° C. for 10 seconds, and 72° C. for 60 seconds; and 1 cycle at 72° C. for 10 minutes. Samples were cooled to 10° C. before removal and further processing.

Five μl of the PCR reaction was analyzed by 1% agarose gel electrophoresis using TAE buffer where an approximately 1700 by product band was observed. The remaining PCR reaction was purified using an ILLUSTRA™ GFX™ PCR DNA and Gel Band Purification Kit according to the manufacturer's instructions.

The fragment was then cloned into Hind III and Bam HI digested pDau109 using an IN-FUSION™ Cloning Kit resulting in plasmid pP244GP. Cloning of the P244GP gene into Hind III-Bam HI digested pDau109 resulted in the transcription of the Aspergillus aculeatus P244GP gene under the control of a NA2-tpi double promoter. NA2-tpi is a modified promoter from the gene encoding the Aspergillus niger neutral alpha-amylase in which the untranslated leader has been replaced by an untranslated leader from the gene encoding the Aspergillus nidulans triose phosphate isomerase.

The cloning protocol was performed according to the IN-FUSION™ Cloning Kit instructions generating a P244GP BBEL construct. The treated plasmid and insert were transformed into Fusion Blue™ E. coli cells (Clontech, Mountain View, Calif., USA) according to the manufacturer's protocol and plated onto LB plates supplemented with 50 μg of ampicillin per ml. After incubating at 37° C. overnight, colonies were seen growing under selection on the LB ampicillin plates. Ten colonies transformed with the pP244GP BBEL construct were cultivated in LB medium supplemented with 50 μg of ampicillin per ml and plasmid was isolated using a FASTPlasmid mini kit from 5Prime (5 PRIME GmbH, Königstrasse 4a, 22767 Hamburg, Germany) according to the manufacturer's instructions.

Isolated plasmids were sequenced with vector primers and in order to determine a representative plasmid expression clone that was free of PCR errors.

The pairs of synthetic oligonucleotide primers shown below were designed to PCR amplify the A. aculeatus CBS 172.66 P2416F BBEL gene, P244GZ BBEL gene, P24169 BBEL gene from the A. aculeatus genomic DNA using the same procedure described above for the P244GP BBEL gene.

P2416 BBEL gene
Primer F-P2416F
5′-ACACAACTGGGGATCCACCATGCTCGCTGTCCTGTCCAT-3′
Primer R-P2416F
5′-AGATCTCGAGAAGCTTATCAGTGCGGCAACCCGTTGT-3′
P244GZ gene
Primer F-P244GZ
5′ACACAACTGGGGATCCACCATGGTTCCTTCAACACCTTCAC-3′
Primer R-P244GZ
5′-AGATCTCGAGAAGCTTATTAAATGCGGCACAACCGCC-3′
P24169 gene
Primer F-P24169
5′-ACACAACTGGGGATCCACCATGAGGGTTCCGTCAGCATT-3′
Primer R-P24169
5′-AGATCTCGAGAAGCTTATTATGCCAATGGAAAGAACTGATTAAAT
CG-3′

Bold letters represent coding sequence. The underlined sequence is homologous to insertion sites of pDau109.

The pairs of synthetic oligonucleotide primers shown below were designed to PCR amplify the P. pinophilum BBEL genes P23WH5 and P23WGQ genes from the P. pinophilum genomic DNA using the same procedure described above for the Aspergillus P244GP BBEL gene.

P23WH5 gene
Primer BBE 10421-forward
5′-ACACAACTGGGGATCCATGGGCAATACGACAAGT-3′
Primer BBE 10421-reverse
5′-GTCACCCTCTAGATCTTTATTGCAGGGGAACGCT-3′
46877_BBE_2399-forward -
5′-ACACAACTGGGGATCCATGCGTTGGTCTGCAGTC-3′
46877_BBE_2399-reverse -
5′-GTCACCCTCTAGATCTTCATGCTTTACATAACCGGC-3′

Bold letters represent coding sequence.

### Example 4

### Characterization of Genomic Sequences Encoding BBEL Polypeptides Having Cellulolytic-Enhancing Activity

DNA sequencing of the BBEL genomic clones was performed with an Applied Biosystems Model 3700 Automated DNA Sequencer using version 3.1 BIG-DYE™ terminator chemistry (Applied Biosystems, Inc., Foster City, Calif., USA) and primer walking strategy. Nucleotide sequence data were scrutinized for quality and all sequences were compared to each other with assistance of PHRED/PHRAP software (University of Washington, Seattle, Wash., USA).

The nucleotide sequence and deduced amino acid sequence of the Aspergillus aculeatus P244GP BBEL gene are shown in SEQ ID NO: 5 and SEQ ID NO: 6, respectively. The coding sequence is 1738 by including the stop codon and is interrupted by introns of 58 by (nucleotides 380 to 437) and 53 by (nucleotides 1050 to 1102). The encoded predicted protein is 508 amino acids. Using the SignalP program (Nielsen et al., 1997, Protein Engineering 10: 1-6), a signal peptide of 20 residues was predicted. The predicted mature protein contains 488 amino acids.

A comparative pairwise global alignment of amino acid sequences was determined using the Needleman and Wunsch algorithm (Needleman and Wunsch, 1970, J. Mol. Biol. 48: 443-453) with gap open penalty of 10, gap extension penalty of 0.5, and the EBLOSUM62 matrix. The alignment showed that the deduced amino acid sequence of the Aspergillus aculeatus gene encoding the P244GP BBEL polypeptide shares 78% identity (excluding gaps) to the deduced amino acid sequence of a BBEL family protein from Aspergillus niger (accession number SWISSPROT: A2QFR3).

The nucleotide sequence and deduced amino acid sequence of the Aspergillus aculeatus P2416F BBEL gene are shown in SEQ ID NO: 7 and SEQ ID NO: 8, respectively. The coding sequence is 1709 bp including the stop codon and contains four introns; 64 bp. (nucleotides 302-365), 65 bp (nucleotides 583-647), 77 bp (1031-1107) and 79 bp (nucleotides 1485-1563) The encoded predicted protein is 474 amino acids. Using the SignalP program (Nielsen et al., 1997, supra), a signal peptide of 13 residues was predicted. The predicted mature protein contains 461 amino acids.

A comparative pairwise global alignment of amino acid sequences was determined using the Needleman and Wunsch algorithm (Needleman and Wunsch, 1970, supra) with gap open penalty of 10, gap extension penalty of 0.5, and the EBLOSUM62 matrix. The alignment showed that the deduced amino acid sequence of the Aspergillus aculeatus gene encoding the P2416F BBEL polypeptide shares 74.26% identity (excluding gaps) to the deduced amino acid sequence of a BBEL family protein from Penicillium chrysogenum (accession number SWISSPROT:B6HR03).

The nucleotide sequence and deduced amino acid sequence of the Aspergillus aculeatus P244GZ BBEL gene are shown in SEQ ID NO: 9 and SEQ ID NO: 10, respectively. The coding sequence is 2037 bp including the stop codon and contains two introns; 55 bp (nucleotides 529-583), and 65 bp (nucleotides 1165-1229). The encoded predicted protein is 638 amino acids. Using the SignalP program (Nielsen et al., 1997, supra), a signal peptide of 20 residues was predicted. The predicted mature protein contains 618 amino acids.

A comparative pairwise global alignment of amino acid sequences was determined using the Needleman and Wunsch algorithm (Needleman and Wunsch, 1970, supra) with gap open penalty of 10, gap extension penalty of 0.5, and the EBLOSUM62 matrix. The alignment showed that the deduced amino acid sequence of the Aspergillus aculeatus gene encoding the P244GZ BBEL polypeptide shares 76% identity (excluding gaps) to the deduced amino acid sequence of a BBEL family protein from Aspergillus niger (accession number SWISSPROT: A2QQL34) The nucleotide sequence and deduced amino acid sequence of the Aspergillus aculeatus

P24169 BBEL gene are shown in SEQ ID NO: 15 and SEQ ID NO: 16, respectively. The coding sequence is 1640 bp including the stop codon and contains two introns; 58 bp (nucleotides 329-368) and 109 bp (nucleotides 1502-1610). The encoded predicted protein is 490 amino acids. Using the SignalP program (Nielsen et al., 1997, supra), a signal peptide of 20 residues was predicted. The predicted mature protein contains 470 amino acids.

A comparative pairwise global alignment of amino acid sequences was determined using the Needleman and Wunsch algorithm (Needleman and Wunsch, 1970, supra) with gap open penalty of 10, gap extension penalty of 0.5, and the EBLOSUM62 matrix. The alignment showed that the deduced amino acid sequence of the Aspergillus aculeatus gene encoding the P24169 BBEL polypeptide shares 46.78% identity (excluding gaps) to the deduced amino acid sequenceS of BBEL family proteins from Aspergillus otyzae (accession number SWISSPROT:Q2UMU8) and Aspergillus flavus (accession number SWISSPROT:B8MMN2).

The nucleotide sequence and deduced amino acid sequence of the Penicillium pinophilum BBE3888 BBEL gene are shown in SEQ ID NO: 1 and SEQ ID NO: 2, respectively. The coding sequence is 1513 bp including the stop codon and contains one intron; 88 bp (nucleotides 260-347). The encoded predicted protein is 474 amino acids. Using the SignalP program (Nielsen et al., 1997, supra), no signal peptide.

A comparative pairwise global alignment of amino acid sequences was determined using the Needleman and Wunsch algorithm (Needleman and Wunsch, 1970, supra) with gap open penalty of 10, gap extension penalty of 0.5, and the EBLOSUM62 matrix. The alignment showed that the deduced amino acid sequence of the P. pinophilum gene encoding the BBE03888BBEL polypeptide shares 92.2% identity (excluding gaps) to the closest homologue.

The nucleotide sequence and deduced amino acid sequence of the Penicillium pinophilum BBE03834 BBEL gene are shown in SEQ ID NO: 3 and SEQ ID NO: 4, respectively. The coding sequence is 1847 bp including the stop codon and contains three introns; 60 bp (nucleotides 249-308), 51 bp (nucleotides 510-560), 50 bp (nucleotides 1630-1679). The encoded predicted protein is 561 amino acids. Using the SignalP program (Nielsen et al., 1997, supra) the peptides is predicted to contain a 20 bp signal peptide. The predicted mature protein contains 541 amino acids.

A comparative pairwise global alignment of amino acid sequences was determined using the Needleman and Wunsch algorithm (Needleman and Wunsch, 1970, supra) with gap open penalty of 10, gap extension penalty of 0.5, and the EBLOSUM62 matrix. The alignment showed that the deduced amino acid sequence of the P. pinophilum gene encoding the BBE03888 BBEL polypeptide shares 92.2% identity (excluding gaps) to the closest homologue.

### Example 5

### Expression of BBEL enzymes gene in Aspergillus oryzae MT3568

One error-free pP244GP clone comprising the P244GP BBEL gene of SEQ ID NO: 5 was selected for further work. Plasmid DNA was then isolated using a JETSTAR 2.0 Plasmid Mini/Midi/Maxi-Protocol (Genomed). The purified plasmid DNA was transformed into protoplasts of Aspergillus oryzae MT3568, which were prepared according to the method of Christensen et al., 1988, Bio/Technology 6: 1419-1422. The selection plates consisted of COVE sucrose+10 mM acetamide+TRITON® X-100 (50 μl/500 ml). The plates were incubated at 37° C. for four days and then 26° C. for one day. The transformation of A. oryzae MT3568 with pP244GP yielded several transformants. Eight transformants were inoculated into separate wells of a 96 microtiter deep well plate (Nunc A/S, Roskilde, Denmark) with each well containing 750 μl of YP+2% glucose medium or 750 μl of YP+2% maltodextrin. The plate was covered with Nunc prescored vinyl sealing tape (ThermoFisher Scientific, Roskilde, Denmark) and incubated at 26° C. stationary for 4 days. The colonies on the selection plates were also restreaked onto COVE sucrose (+10 mM acetamide+TRITON® X-100 (50μ/500 ml)). The plates were incubated at 37° C. and this selection procedure was repeated in order to stabilize the transformants.

Several Aspergillus oryzae transformants produced the recombinant P244GP BBEL polypeptide of SEQ ID NO: 6 as judged by SDS-PAGE analysis.

Purified plasmid for error-free clones of plasmids pP2416F, pP244GZ, pP24169, pP23WGQ and pP23WH5 were isolated. pP2416F comprising the P2416F BBEL gene, pP244GZ comprising the P244GZ BBEL gene, pP24169 comprising the P24169 BBEL gene pP23WGQ comprising BBE3834 BBEL gene and pP23WH5 comprising BBE3888 BBEL gene, were transformed into A. oryzae MT3568 using the same procedure above.

One Aspergillus oryzae transformant for each of the P244GP BBEL gene (EXP03889), P2416F BBEL gene (EXP03824), P23WH5 BBEL gene (EXP3888), P244GZ BBEL gene (EXP03887), and P24169 BBEL gene (EXP03834) were streaked onto PDA plates and incubated at 34° C. for two weeks. Spores were removed in 5 ml of 0.01% TWEEN® 20 and 4 ml of spore suspension was inoculated into 500 ml of YPG (YP+2% glucose), and incubated at 28° C. for 4 days with shaking at 250 rpm. The supernatants were harvested by filtering through a 0.22 μm filter.

### Example 6

### Berberine Bridge Enzyme Like (BBEL) Enzymes Boosting Hydrolysis of Pretreated Corn Stover (PCS)

The effect of BBEL on the cellulolytic activity of a cellulase composition was evaluated according to the procedures described below.

A blend of an Aspergillus fumigatus GH10 xylanase (WO 2006/078256) and Aspergillus fumigatus beta-xylosidase (WO 2011/057140) with a Trichoderma reesei cellulase preparation containing Aspergillus fumigatus cellobiohydrolase I (WO 2011/057140), Aspergillus fumigatus cellobiohydrolase II (WO 2011/057140), Aspergillus fumigatus beta-glucosidase variant (PCT/US2011/054185), and Penicillium sp. GH61 polypeptide (WO 2011/041397) was used as the cellulase preparation.

Un-washed PCS (uwPCS): Corn stover was pretreated at the U.S. Department of Energy National Renewable Energy Laboratory (NREL) using dilute sulfuric acid. Pretreated corn stover was produced at conditions of 190° C., 1 min residence time, 0.05 g acid/g dry biomass, and at a 30% total solid concentration. The water-insoluble solids in the pretreated corn stover (PCS) contained 52.93% cellulose, 2.43% hemicellulose and 31.77% lignin. Cellulose and hemicelluloses in PCS were determined by a two-stage sulfuric acid hydrolysis with subsequent analysis of sugars by high performance liquid chromatography using NREL Standard Analytical Procedure #002. Lignin was determined gravimetrically after hydrolyzing the cellulose and hemicellulose fractions with sulfuric acid using NREL Standard Analytical Procedure #003. See http://www.nrel.gov/biomass/analytical_procedures.html.

uwPCS was used for hydrolysis/saccharification at initial total solid (TS) of 12.6% and total weight of 20 g. The pH was adjusted to 5.0 by using 10 M sodium hydroxide. Cellulase composition as described above was added into uwPCS as control with a ratio of the cellulase composition to cellulose of 0.5% (w/w). Then, BBELs with final concentrations of 10 μg, 25 μg and 50 μg/g cellulose were added into hydrolysis/saccharification system on top of the cellulase composition. The effects of BBELs were evaluated based on the control, respectively. The flasks were incubated at 50° C. for 72 hours, with shaking at 130 rpm. All experiments were performed in triplicates. After hydrolysis/saccharification was completed, the sugar was analyzed by High Performance Liquid Chromatography (HPLC).

For HPLC measurement, the collected samples were filtered using 0.22 μm syringe filters (Millipore, Bedford, Mass., USA) and the filtrates were analyzed for sugar content as described below. The sugar concentrations of samples diluted in 0.005 M H₂SO₄ were measured using a 7.8×300 mm AMINEX® HPX-87H column (Bio-Rad Laboratories, Inc., Hercules, Calif., USA) by elution with 0.005 M H₂SO₄ at 65° C. at a flow rate of 0.7 ml per minute, and quantification by integration of the glucose signal from refractive index detection (CHEMSTATION®, AGILENT® 1100 HPLC, Agilent Technologies, Santa Clara, Calif., USA) calibrated by pure sugar samples. The resultant glucose was used to calculate the percentage of glucose yield from glucans for each reaction. Measured sugar concentrations were adjusted for the appropriate dilution factor. The net concentrations of enzymatically-produced sugars were determined by adjusting the measured sugar concentrations for corresponding background sugar concentrations in unwashed biomass at zero time point. All HPLC data processing was performed using MICROSOFT EXCEL™ software (Microsoft, Richland, Wash., USA).

The degree of cellulose conversion to glucose was calculated according to the following publication: Zhu, Y., et al. 2010, Bioresource Technology. 102(3): 2897-2903.

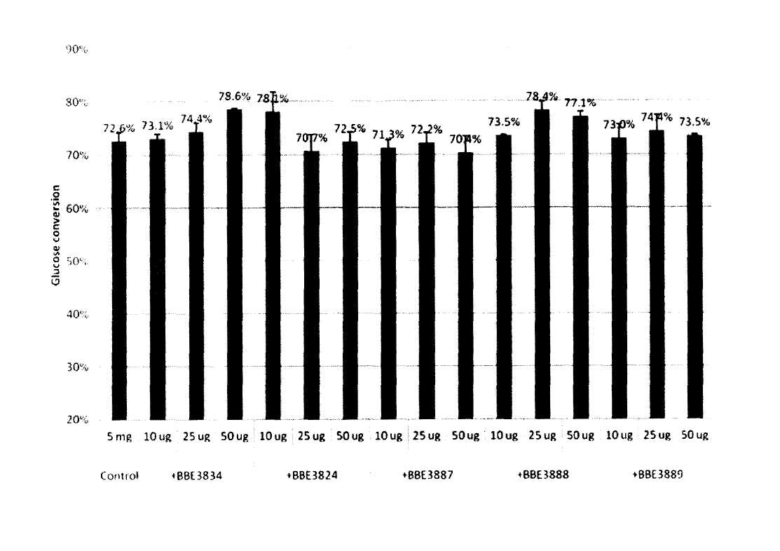

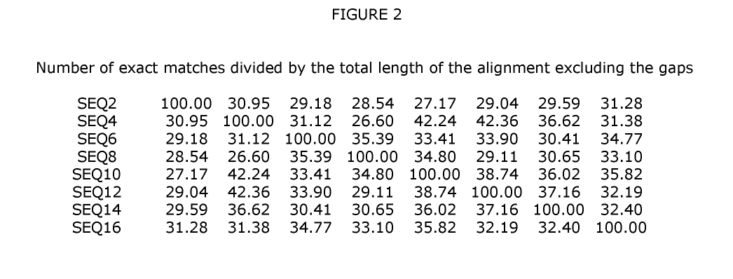

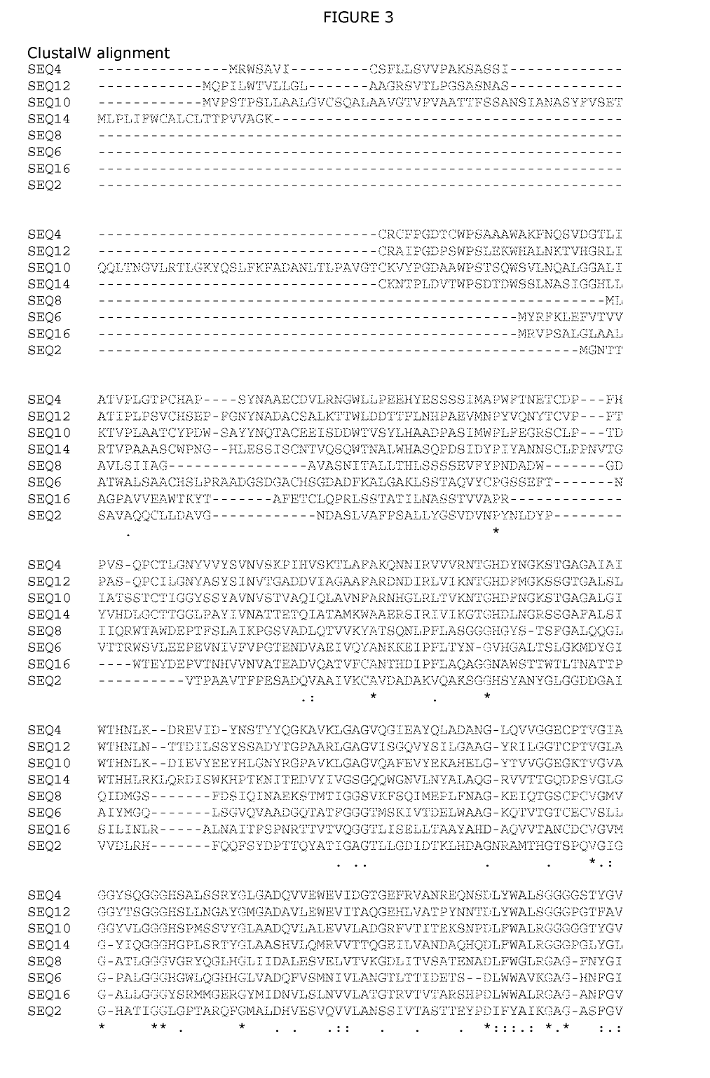

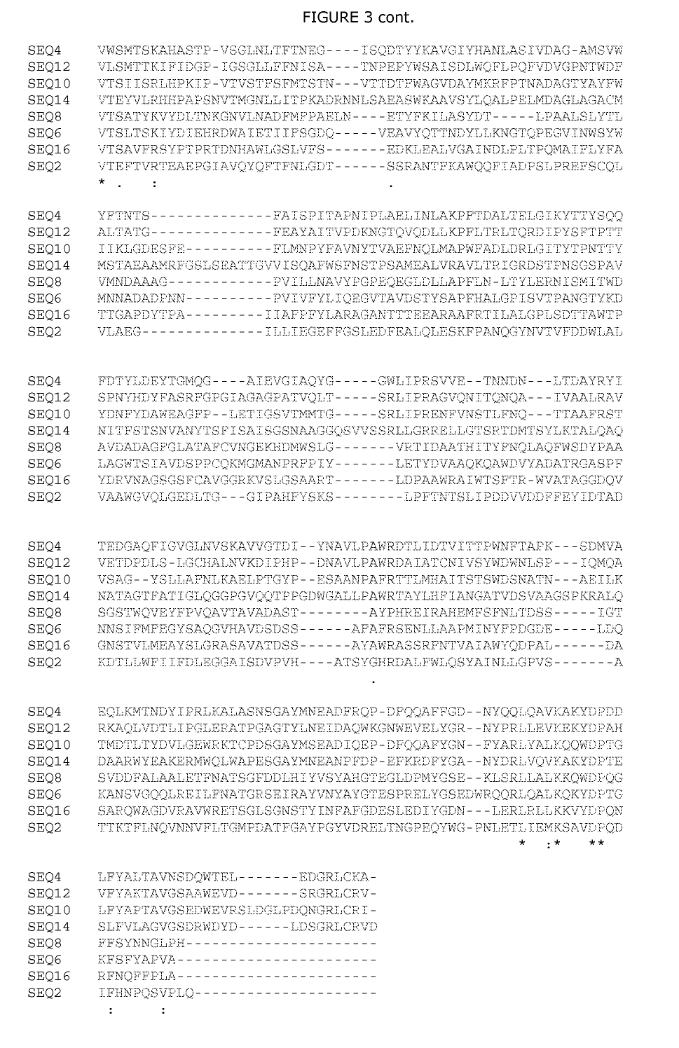

The conversion of uwPCS to glucose after 72-hour hydrolysis/saccharification was shown in Table 1 below and in FIG. 1.

TABLE 1
Cellulolytic enhancing activity of BBELs on
hydrolysis/saccharification of un-washed PCS.

| | | BBEL 3834 | | | BBEL 3888 | | | BBEL 3889 | | |
|---|---|---|---|---|---|---|---|---|---|---|
| | | (μg/g cellulose) | | | (μg/g cellulose) | | | (μg/g cellulose) | | |
| | Control | 10 | 25 | 50 | 10 | 25 | 50 | 10 | 25 | 50 |
| Glucose | 72.6 | 73.1 | 74.4 | 78.6 | 73.5 | 78.4 | 77.1 | 73.0 | 74.4 | 73.5 |
| conversion (%) | | | | | | | | | | |

As shown in Table 1 and FIG. 1, when BBELs added, uwPCS conversion to glucose was improved considerably.

The invention described and claimed herein is not to be limited in scope by the specific aspects herein disclosed, since these aspects are intended as illustrations of several aspects of the invention. Any equivalent aspects are intended to be within the scope of this invention. Indeed, various modifications of the invention in addition to those shown and described herein will become apparent to those skilled in the art from the foregoing description. Such modifications are also intended to fall within the scope of the appended claims. In the case of conflict, the present disclosure including definitions will control.

### EMBODIMENTS

A related aspect of the invention relates to the following embodiments:

### Embodiment 1

An enzyme composition comprising a cellulolytic preparation and a Berberine Bridge Enzyme Like (BBEL) enzyme.

### Embodiment 2

The enzyme composition of embodiment 1, wherein the cellulolytic preparation is derived from Trichoderma reesei, Humicola insolens or Chrysosporium lucknowense.

### Embodiment 3

The enzyme composition of any one of embodiment 1 or 2, wherein the cellulolytic preparation comprises a beta-glucosidase, preferably one derived from a strain of the genus Aspergillus, such as Aspergillus oryzae, such as the one disclosed in WO 2002/095014 or the fusion protein having beta-glucosidase activity disclosed in WO 2008/057637, or Aspergillus fumigatus, such as such as one disclosed in WO 2005/047499 or an Aspergillus fumigatus beta-glucosidase variant disclosed in co-pending U.S. provisional application No. 61/388,997; or a strain of the genus a strain Penicillium, such as a strain of the Penicillium brasilianum disclosed in WO 2007/019442, or a strain of the genus Trichoderma, such as a strain of Trichoderma reesei.

### Embodiment 4

The enzyme composition of any one of embodiments 1-3, wherein the cellulolytic preparation comprises a GH61 polypeptide having cellulolytic enhancing activity such as one derived from the genus Thermoascus, such as a strain of Thermoascus aurantiacus, such as the one described in WO 2005/074656 as SEQ ID NO: 2; or one derived from the genus Thielavia, such as a strain of Thielavia terrestris, such as the one described in WO 2005/074647 as SEQ ID NO: 7 and SEQ ID NO: 8; or one derived from a strain of Aspergillus, such as a strain of Aspergillus fumigates, such as the one described in WO 2010/138754 as SEQ ID NO: 1 and SEQ ID NO: 2; or one derived from a strain derived from Penicillium, such as a strain of Penicillium emersonii, such as the one disclosed in WO 2011/041397.

### Embodiment 5

The enzyme composition of any one of embodiments 1-4, wherein the cellulolytic preparation comprises a xylanase, preferably a GH10 xylanase, such as one derived from a strain of the genus Aspergillus, such as a strain from Aspergillus fumigatus, such as the one disclosed as Xyl III in WO 2006/078256, or Aspergillus aculeatus, such as the one disclosed in WO 94/21785 as SEQ ID NO: 5 (Xyl II).

### Embodiment 6

The enzyme composition of any one of embodiments 1-5, wherein the cellulolytic preparation comprises a beta-xylosidase, such as one derived from a strain of the genus Aspergillus, such as a strain of Aspergillus fumigatus, such as the one disclosed in co-pending US provisional No. 61/526,833, or derived from a strain of Trichoderma, such as a strain of Trichoderma reesei, such as the mature polypeptide of SEQ ID NO: 58 in WO 2011/057140.

### Embodiment 7

The enzyme composition of any one of embodiments 1-6, wherein the cellulolytic preparation comprises a cellobiohydrolase I (CBH I), such as one derived from a strain of the genus Aspergillus, such as a strain of Aspergillus fumigatus, such as the Cel7a CBHI disclosed in SEQ ID NO: 2 in WO 2011/057140, or a strain of the genus Trichoderma, such as a strain of Trichoderma reesei.

### Embodiment 8

The enzyme composition of any one of embodiments 1-7, wherein the cellulolytic preparation comprises a cellobiohydrolase II (CBH II, such as one derived from a strain of the genus Aspergillus, such as a strain of Aspergillus fumigates; or a strain of the genus Trichoderma, such as Trichoderma reesei, or a strain of the genus Thielavia, such as a strain of Thielavia terrestris, such as cellobiohydrolase II CEL6A from Thielavia terrestris.

### Embodiment 9

The enzyme composition of any one of embodiments 1-8, wherein the Berberine Bridge Enzyme Like (BBEL) enzyme is derived from a strain of the genus Aspergillus, such as a strain of Aspergillus aculeatus, such as Aspergillus aculeatus CBS 172.66 or a Berberine Bridge Enzyme Like (BBEL) enzyme having at least 80% sequence identity to the mature polypeptide of any of SEQ ID NO: 6, SEQ ID NO: 8 or SEQ ID NO: 10, at least 75% sequence identity to the mature polypeptide of any of SEQ ID NO: 12 or SEQ ID NO:14, or at least 60% sequence identity to the mature polypeptide of SEQ ID NO: 16 herein.

### Embodiment 10

The enzyme composition of any one of embodiments 1-8, wherein the Berberine Bridge Enzyme Like (BBEL) enzyme is derived from a strain of the genus Penicillium, such as a strain of Penicillium pinophilum or a Berberine Bridge Enzyme Like (BBEL) enzyme having at least 95% sequence identity to the mature polypeptide of SEQ ID NO: 2 or at least 90% sequence identity to the mature polypeptide of SEQ ID NO: 4 herein.

### Embodiment 11

The enzyme composition of any one of embodiments 1-10, wherein the cellulolytic preparation comprises a GH61 polypeptide having cellulolytic enhancing activity and a beta-glucosidase.

### Embodiment 12

The enzyme composition of any one of embodiments 1-10, wherein the cellulolytic preparation comprises a GH61 polypeptide having cellulolytic enhancing activity, a beta-glucosidase, and a xylanase.

### Embodiment 13

The enzyme composition of any one of embodiments 1-10, wherein the cellulolytic preparation comprises a GH61 polypeptide having cellulolytic enhancing activity, a beta-glucosidase, a xylanase and a beta-xylosidase.

### Embodiment 14

The enzyme composition of any one of embodiments 1-10, wherein the cellulolytic preparation comprises a GH61 polypeptide having cellulolytic enhancing activity, a beta-glucosidase, a xylanase, a beta-xylosidase, and a CBHI.

### Embodiment 15

The enzyme composition of any one of embodiments 1-10, wherein the cellulolytic preparation comprises a GH61 polypeptide having cellulolytic enhancing activity, a beta-glucosidase, a xylanase, a beta-xylosidase, a CBHI and a CBHII.

### Embodiment 16

The enzyme composition of any of embodiments 1-15, wherein the cellulolytic preparation is a Trichoderma reesei cellulolytic preparation, further comprising Thermoascus aurantiacus GH61A polypeptide having cellulolytic enhancing activity (SEQ ID NO: 2 in WO 2005/074656), Aspergillus otyzae beta-glucosidase fusion protein (WO 2008/057637), and Aspergillus aculeatus xylanase (Xyl II in WO 94/21785).

### Embodiment 17

The enzyme composition of any of embodiments 1-15, wherein the cellulolytic preparation is a Trichoderma reesei cellulolytic preparation, further comprising Thermoascus aurantiacus GH61A polypeptide having cellulolytic enhancing activity (SEQ ID NO: 2 in WO 2005/074656), Aspergillus fumigatus beta-glucosidase (SEQ ID NO: 2 of WO 2005/047499) and Aspergillus aculeatus xylanase (Xyl II disclosed in WO 94/21785).

### Embodiment 18

The enzyme composition of any one of embodiments 1-15, wherein the cellulolytic preparation is a Trichoderma reesei cellulolytic preparation further comprising Thermoascus aurantiacus GH61A polypeptide having cellulolytic enhancing activity (SEQ ID NO: 2 in WO 2005/074656), Aspergillus fumigatus beta-glucosidase (SEQ ID NO: 2 of WO 2005/047499) and Aspergillus aculeatus xylanase (Xyl II disclosed in WO 94/21785) and the acetylxylan esterase (AXE) is the one derived from Thielavia terrestris disclosed in WO 2009/042846 as SEQ ID NO: 2 or SEQ ID NO: 1 herein or an acetylxylan esterase having at least 80%, such as at least 85%, such as at least 90%, preferably at least 95%, such as at least 96%, such as at least 97%, such as at least 98%, such as at least 99% identity to SEQ ID NO: 2 in WO 2009/042846 SEQ ID NO: 1 herein.

### Embodiment 19

The enzyme composition of any one of embodiments 1-15, wherein the cellulolytic preparation is a Trichoderma reesei cellulolytic preparation further comprising Penicillium emersonii GH61A polypeptide having cellulolytic enhancing activity disclosed in WO 2011/041397, Aspergillus fumigatus beta-glucosidase (SEQ ID NO: 2 of WO 2005/047499) and Aspergillus fumigatus xylanase (Xyl III in WO 2006/078256) and the acetylxylan esterase (AXE) is the one derived from Thielavia terrestris disclosed in WO 2009/042846 as SEQ ID NO: 2 or SEQ ID NO: 1 or an acetylxylan esterase having at least 80%, such as at least 85%, such as at least 90%, preferably at least 95%, such as at least 96%, such as at least 97%, such as at least 98%, such as at least 99% identity to SEQ ID NO: 2 in WO 2009/042846 SEQ ID NO: 1.

### Embodiment 20

The enzyme composition of any one of embodiments 1-19, wherein the ratio between cellulolytic preparation and a Berberine Bridge Enzyme Like (BBEL) enzyme is in the range between 500:1 and 100:1, such as between 50:1 and 2:1, such as around 4:1.

### Embodiment 21

A method of hydrolyzing cellulosic material, comprising subjecting the cellulosic material to a cellulolytic preparation and a berberine bridge enzymes like (BBEL) enzyme.

### Embodiment 22

The method of embodiment 21, wherein cellulosic material is pretreated cellulosic material.

### Embodiment 23

The method of embodiment 21 or 22, wherein the cellulosic material is plant material chips, plant stem segments and/or whole plant stems.

### Embodiment 24

The method of embodiment 23, wherein cellulosic material is selected from the group comprising arundo, bagasse, bamboo, corn cob, corn fiber, corn stover, miscanthus, orange peel, rice straw, switchgrass, wheat straw.

### Embodiment 25

The method of embodiment 24, wherein the source of the cellulosic material is corn stover, corn cobs, and/or wheat straw.

### Embodiment 26

The method of any one of embodiments 22-25, wherein the pretreating cellulosic material is pretreated by chemical pretreatment, a physical pretreatment, or a chemical pretreatment and a physical pretreatment.

### Embodiment 27

The method of any one of embodiments 22-26, wherein the cellulosic material is thermomechanically pulped plant material.

### Embodiment 28

The method of any one of embodiments 22-27, wherein the acetylated cellulosic material is thermomechanically pulped plant material, such as acetylated corn stover pulp.

### Embodiment 29

The method of any one of embodiments 22-28, wherein pretreating the cellulosic material includes pretreatment with an acid.

### Embodiment 30

The method of any one of embodiments 22-29, wherein the cellulosic material has been prepared by pretreating cellulosic material at high temperature, high pressure with an acid.

### Embodiment 31

The method of embodiment 29 or 30, wherein acid pretreatment is carried out using acetic acid.

### Embodiment 32

The method of any one of embodiments 22-31, wherein the acetylated cellulosic material has been prepared by pretreating cellulosic material using organosolv pretreatement, such as Acetosolv and Acetocell processes.

### Embodiment 33

The method of any one of embodiments 22-32, wherein the soluble fractions containing sugars, acid and solubilized lignin is removed from the cellulosic material after pretreatment.

### Embodiment 34

The method of any one of embodiments 22-33, wherein hydrolysis is carried out at a temperature between 20-70° C., such as 30-60° C., preferably 45-55° C. at a pH in the range 4-6, such as 4.5-5.5.

### Embodiment 35

The method of any one of embodiment 22-34, wherein the cellulosic material is present at 1-20 (w/w) % of TS, such as 2-10 (w/w) % TS, such as around 5 (w/w) % TS.

### Embodiment 36

The method of any one of embodiments 22-35, wherein the hydrolysis is carried out for 1-20 days, preferably between from 3-15 days.

### Embodiment 37

The method of any of embodiments 22-36, wherein the cellulolytic preparation is derived from Trichoderma reesei, Humicola insolens or Chrysosporium lucknowense.

### Embodiment 38

The method of any one of embodiments 22-37, wherein the cellulolytic preparation comprises a beta-glucosidase, preferably one derived from a strain of the genus Aspergillus, such as Aspergillus oryzae, such as the one disclosed in WO 2002/095014 or the fusion protein having beta-glucosidase activity disclosed in WO 2008/057637, or Aspergillus fumigatus, such as such as one disclosed in WO 2005/047499 or an Aspergillus fumigatus beta-glucosidase variant disclosed in co-pending US provisional application #61/388,997; or a strain of the genus a strain Penicillium, such as a strain of the Penicillium brasilianum disclosed in WO 2007/019442, or a strain of the genus Trichoderma, such as a strain of Trichoderma reesei.

### Embodiment 39

The method of any one of embodiments 22-38, wherein the cellulolytic preparation comprises a GH61 polypeptide having cellulolytic enhancing activity, such as one derived from the genus Thermoascus, such as a strain of Thermoascus aurantiacus, such as the one described in WO 2005/074656 as SEQ ID NO: 2; or one derived from the genus Thielavia, such as a strain of Thielavia terrestris, such as the one described in WO 2005/074647 as SEQ ID NO: 7 and SEQ ID NO: 8; or one derived from a strain of Aspergillus, such as a strain of Aspergillus fumigates, such as the one as described in WO 2010/138754 as SEQ ID NO: 1 and SEQ ID NO: 2; or one derived from a strain derived from Penicillium, such as a strain of Penicillium emersonii, such as the one disclosed in WO 2011/041397.

### Embodiment 40

The method of any one of embodiments 22-39, wherein the cellulolytic preparation comprises a xylanase, preferably a GH10 xylanase, such as one derived from a strain of the genus Aspergillus, such as a strain from Aspergillus fumigatus, such as the one disclosed as Xyl III in WO 2006/078256, or Aspergillus aculeatus, such as the one disclosed in WO 94/21785 as SEQ ID NO: 5 (Xyl II).

### Embodiment 41

The method of any one of embodiments 22-39, wherein the cellulolytic preparation comprises a beta-xylosidase, such as one derived from a strain of the genus Aspergillus, such as a strain of Aspergillus fumigatus, such as the one disclosed in co-pending US provisional #61/526833, or derived from a strain of Trichoderma, such as a strain of Trichoderma reesei, such as the mature polypeptide of SEQ 10 NO: 58 in WO 2011/057140.

### Embodiment 42

The method of any one of embodiments 22-41, wherein the berberine bridge (BBEL) is derived from a strain of the genus Aspergillus, such as a strain of Aspergillus aculeatus, such as Aspergillus aculeatus CBS 172.66 or a Berberine Bridge Enzyme Like (BBEL) having at least 80% sequence identity to the mature polypeptide of any of SEQ ID NO: 6, SEQ ID NO: 8 or SEQ ID NO: 10, at least 75% sequence identity to the mature polypeptide of any of SEQ ID NO: 12 or SEQ ID NO:14, or at least 60% sequence identity to the mature polypeptide of SEQ ID NO: 16 herein.

### Embodiment 43

The method of any one of embodiments 22-41, wherein the Berberine Bridge Enzyme Like (BBEL) enzyme is derived from a strain of the genus Penicillium, such as a strain of Penicillium pinophilum or an Berberine Bridge Enzyme Like (BBEL) enzyme having at least 95% sequence identity to the mature polypeptide of SEQ ID NO: 2 or at least 90% sequence identity to the mature polypeptide of SEQ ID NO: 4 herein.

### Embodiment 44

The method of any one of embodiments 22-43, wherein the cellulolytic preparation comprises a cellobiohydrolase I (CBHI), such as one derived from a strain of the genus Aspergillus, such as a strain of Aspergillus fumigatus, or a strain of the genus Trichoderma, such as a strain of Trichoderma reesei.

### Embodiment 45

The method of any one of embodiments 22-45, wherein the cellulolytic preparation comprises a cellobiohydrolase II (CBHII), such as one derived from Aspergillus fumigates; or a strain of the genus Trichoderma, such as Trichoderma reesei, or a strain of the genus Thielavia, such as a strain of Thielavia terrestris, such as cellobiohydrolase II CEL6A from Thielavia terrestris.

### Embodiment 46

The method of any one of embodiments 22-45, wherein the cellulolytic preparation comprises a GH61 polypeptide having cellulolytic enhancing activity and a beta-glucanase.

### Embodiment 47

The method of any one of embodiments 22-45, wherein the cellulolytic preparation comprises a GH61 polypeptide having cellulolytic enhancing activity, a beta-glucanase, and a xylanase.

### Embodiment 48

The method of any one of embodiments 22-45, wherein the cellulolytic preparation comprises a GH61 polypeptide having cellulolytic enhancing activity, a beta-glucanase, a xylanase and a beta-xylosidase.

### Embodiment 49

The method of any one of embodiments 22-45, wherein the cellulolytic preparation comprises a GH61 polypeptide having cellulolytic enhancing activity, a beta-glucanase, a xylanase, a beta-xylosidase, and a CBHI.

### Embodiment 50

The method of any one of embodiments 22-45, wherein the cellulolytic preparation comprises a GH61 polypeptide having cellulolytic enhancing activity, a beta-glucanase, a xylanase, a beta-xylosidase, a CBHI and a CBHII.

### Embodiment 51

The method of any of embodiments 22-50, wherein the cellulolytic preparation is a Trichoderma reesei cellulolytic preparation, further comprising Thermoascus aurantiacus GH61A polypeptide having cellulolytic enhancing activity (SEQ ID NO: 1 and SEQ ID NO: 2 in WO 2005/074656), Aspergillus otyzae beta-glucosidase fusion protein (WO 2008/057637), and Aspergillus aculeatus xylanase (Xyl II in WO 94/21785).

### Embodiment 52

The method of any of embodiments 22-51, wherein the cellulolytic preparation is a Trichoderma reesei cellulolytic preparation, further comprising Thermoascus aurantiacus GH61A polypeptide having cellulolytic enhancing activity (SEQ ID NO: 2 in WO 2005/074656), Aspergillus fumigatus beta-glucosidase (SEQ ID NO: 2 of WO 2005/047499) and Aspergillus aculeatus xylanase (Xyl II disclosed in WO 94/21785).

### Embodiment 53

The method of any of embodiments 22-52, wherein the cellulolytic preparation is a Trichoderma reesei cellulolytic preparation, further comprising Penicillium emersonii GH61A polypeptide having cellulolytic enhancing activity (SEQ ID NO: 2 in WO 2011/04139, Aspergillus fumigatus beta-glucosidase variant (disclosed in U.S. provisional application No. 61/388,997 and Aspergillus fumigatus xylanase (Xyl III disclosed in WO 2006/078256) and beta-xylosidase derived from a strain of Aspergillus fumigatus.

### Embodiment 54

The method of any of embodiments 22-53, wherein the cellulolytic preparation is added in amounts of about 0.01 to about 50.0 mg, e.g., about 1 to about 25 mg, such as about 2-10 mg, such as about 4 to about 8 mg protein per gram cellulosic material.

### Embodiment 55

The method of any of embodiments 22-54, wherein the Berberine Bridge Enzyme Like (BBEL) is used in amounts of 0.001 to about 10 mg, such as 0.005 to about 1 mg, such as 0.01 to about 0.05 mg enzyme protein of the cellulosic material.

### Embodiment 56

The method of any one of embodiments 22-55, wherein the ratio between cellulolytic preparation and berberine bridge enzymes (BBEL) is in the range of a enzyme protein weight ratio of between 1000:1 and 10:1, such as between 500:1 and 100:1, such as about 250:1.

### Embodiment 57

The method of any one of embodiments 22-56, wherein the cellulolytic preparation is a Trichoderma reesei cellulolytic preparation further comprising Thermoascus aurantiacus GH61A polypeptide having cellulolytic enhancing activity (SEQ ID NO: 1 and SEQ ID NO: 2 in WO 2005/074656), Aspergillus fumigatus beta-glucosidase (SEQ ID NO: 2 of WO 2005/047499) and Aspergillus aculeatus xylanase (Xyl II disclosed in WO 94/21785), further comprising Berberine Bridge Enzyme Like (BBEL) enzymes derived from Thielavia terrestris having at least 80% sequence identity to the mature polypeptide of any of SEQ ID NO: 6, SEQ ID NO: 8 or SEQ ID NO: 10, at least 75% sequence identity to the mature polypeptide of any of SEQ ID NO: 12 or SEQ ID NO:14, or at least 60% sequence identity to the mature polypeptide of SEQ ID NO: 16.

### Embodiment 58

The method of any one of embodiments 22-56, wherein the cellulolytic preparation is a Trichoderma reesei cellulolytic preparation further comprising Thermoascus aurantiacus GH61A polypeptide having cellulolytic enhancing activity (SEQ ID NO: 1 and SEQ ID NO: 2 in WO 2005/074656), Aspergillus fumigatus beta-glucosidase (SEQ ID NO: 2 of WO 2005/047499) and Aspergillus aculeatus xylanase (Xyl II disclosed in WO 94/21785), further comprising and the Berberine Bridge Enzyme Like (BBEL) enzymes derived from Thielavia terrestris having at least 80% sequence identity to the mature polypeptide of any of SEQ ID NO: 6, SEQ ID NO: 8 or SEQ ID NO: 10, at least 75% sequence identity to the mature polypeptide of any of SEQ ID NO: 12 or SEQ ID NO:14, or at least 60% sequence identity to the mature polypeptide of SEQ ID NO: 16.

### Embodiment 59

The method of any one of embodiments 22-57, wherein the cellulolytic preparation is a Trichoderma reesei cellulolytic preparation further comprising Penicillium emersonii GH61A polypeptide having cellulolytic enhancing activity disclosed in WO 2011/041397, Aspergillus fumigatus beta-glucosidase variant (disclosed in co-pending US provisional application #61/388,997, Aspergillus fumigatus xylanase (Xyl III in WO 2006/078256), further comprising a Berberine Bridge Enzyme Like (BBEL) enzymes derived from Aspergillus aculeatus having at least 95% sequence identity to the mature polypeptide of SEQ ID NO: 2, at least 90% sequence identity to the mature polypeptide of SEQ ID NO: 4 as disclosed herein.

### Embodiment 60

The method of any one of embodiments 22-57, wherein the cellulolytic preparation is a Trichoderma reesei cellulolytic preparation further comprising Penicillium emersonii GH61A polypeptide having cellulolytic enhancing activity disclosed in WO 2011/041397, Aspergillus fumigatus beta-glucosidase variant (disclosed in co-pending US provisional application No. 61/388,997, Aspergillus fumigatus xylanase (Xyl III in WO 2006/078256), further comprising the Berberine Bridge Enzyme Like (BBEL) enzymes derived from Aspergillus aculeatus having at least 95% sequence identity to the mature polypeptide of SEQ ID NO: 2, at least 90% sequence identity to the mature polypeptide of SEQ ID NO: 4 as disclosed herein.

### Embodiment 61

A process of producing a fermentation product from cellulosic material, comprising:
(a) hydrolyzing said cellulosic material by subjecting the material to an enzyme composition according to any one of embodiments 1-21 or according to any one of the hydrolysis method embodiments in any one of embodiments 22-60;
(b) fermenting using a fermenting organism; and
(c) optionally recovering the fermentation product.

### Embodiment 62

The process of embodiment 61, wherein the fermentation product is ethanol.

### Embodiment 63

The process of embodiment 62 or 62, wherein the fermenting organism is a yeast, such as strain of the genus Saccharomyces, such as a strain of Saccharomyce cerevisie.

### Embodiment 64

The process of any one of embodiments 61-63, wherein the cellulosic material is corn stover pulp.

### Embodiment 65

An isolated polypeptide having Berberine Bridge Enzyme Like (BBEL) enzymatic activity selected from the group consisting of:
(a) a polypeptide having at least 95% sequence identity to the mature polypeptide of SEQ ID NO: 2, at least 90% sequence identity to the mature polypeptide of SEQ ID NO: 4, at least 80% sequence identity to the mature polypeptide of any of SEQ ID NO: 6, SEQ ID NO: 8 or SEQ ID NO: 10, at least 75% sequence identity to the mature polypeptide of any of SEQ ID NO: 12 or SEQ ID NO:14, or at least 60% sequence identity to the mature polypeptide of SEQ ID NO: 16;
(b) a polypeptide encoded by a polynucleotide that hybridizes under medium, medium-high, high, or very high stringency conditions with
(i) the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15,
(ii) the cDNA sequence thereof or
(iii) the full-length complement of (i) or (ii);

(c) a polypeptide encoded by a polynucleotide having at least 95% sequence identity to the mature polypeptide of SEQ ID NO: 1, at least 90% sequence identity to the mature polypeptide coding sequence of SEQ ID NO: 3, at least 80% sequence identity to the mature polypeptide coding sequence of any of SEQ ID NO: 5, SEQ ID NO: 7 or SEQ ID NO: 9, at least 75% sequence identity to the mature polypeptide coding sequence of any of SEQ ID NO: 11 or SEQ ID NO:13, or at least 60% sequence identity to the mature polypeptide coding sequence of SEQ ID NO: 15
(d) a variant of the mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16, comprising a substitution, deletion, and/or insertion at one or more (e.g., several) positions; and
(e) a fragment of the polypeptide of (a), (b), (c), or (d) that has Berberine Bridge Enzyme Like enzyme activity.

### Embodiment 66

The polypeptide of embodiment 65, having at least 95%, at least 96%, at least 97%, at least
98%, at least 99% or 100% sequence identity to the mature polypeptide of SEQ ID NO: 2, at least 90%, at least 91%, at least 92%, at least 93%, at least 94%, at least 95%, at least 96%, at least 97%, at least 98%, at least 99% or 100% sequence identity to the mature polypeptide of SEQ ID NO: 4, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 6, SEQ ID NO: 8 or SEQ ID NO:10, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 12 or SEQ ID NO: 2, or at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99% or 100% sequence identity to the mature polypeptide of SEQ ID NO: 16.

### Embodiment 67

The polypeptide of embodiment 65 or 66, which is encoded by a polynucleotide that hybridizes under medium stringency conditions, medium-high stringency conditions, high stringency conditions, or very high stringency conditions with (i) the mature polypeptide coding sequence of SEQ ID NO: 1, SEQ ID NO: 3, SEQ ID NO: 5, SEQ ID NO: 7, SEQ ID NO: 9, SEQ ID NO: 11, SEQ ID NO: 13 or SEQ ID NO: 15, (ii) the cDNA sequence thereof, or (iii) the full-length complement of (i) or (ii).

### Embodiment 68

The polypeptide of any of embodiments 65-67, which is encoded by a polynucleotide having at least 95% sequence identity to the mature polypeptide of SEQ ID NO: 1, at least 90% sequence identity to the mature polypeptide of SEQ ID NO: 3, at least 80% sequence identity to the mature polypeptide of any of SEQ ID NO: 5, SEQ ID NO: 7 or SEQ ID NO: 9, at least 75% sequence identity to the mature polypeptide of any of SEQ ID NO: 11 or SEQ ID NO:13, or at least 60% sequence identity to the mature polypeptide of SEQ ID NO: 15 or the cDNA sequence thereof.

### Embodiment 69

The polypeptide of any of embodiments 65-68, comprising or consisting of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16, or the mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16.

### Embodiment 70

The polypeptide of embodiment 69, wherein the mature polypeptide is amino acids 29 to 471 of SEQ ID NO: 2, 21 to 561 of SEQ ID NO: 4, 21 to 508 of SEQ ID NO: 6, 14 to 474 of SEQ ID NO: 8, 21 to 638 of SEQ ID NO: 10, 20 to 577 of SEQ ID NO: 12, 19 to 607 of SEQ ID NO: 14 or 21 to 490 of SEQ ID NO: 16.

### Embodiment 71

The polypeptide of any of embodiments 65-68, which is a variant of the mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16 comprising a substitution, deletion, and/or insertion at one or more positions.

### Embodiment 72

The polypeptide of embodiment 65, which is a fragment of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16, wherein the fragment has Berberine Bridge Enzyme Like activity.

### Embodiment 73

An isolated polypeptide comprising a catalytic domain selected from the group consisting of: (a) a catalytic domain having at least 60% sequence identity to amino acids 428 to 472 of SEQ ID NO: 2, 502 to 541 of SEQ ID NO: 4, 463 to 503 of SEQ ID NO: 6, 431 to 473 of SEQ ID NO: 8, 572 to 615 of SEQ ID NO: 10, 517 to 561 of SEQ ID NO: 12, 546 to 581 of SEQ ID NO: 14 or 448 to 484 of SEQ ID NO: 16;
(b) a catalytic domain encoded by a polynucleotide that hybridizes under medium, medium-high, high, or very high stringency conditions with
   (i) nucleotides 1370 to 1504 of SEQ ID NO: 1, nucleotides 1615 to 1629 and/or 1680 to 1784 of SEQ ID NO: 3, nucleotides 1498 to 1620 of SEQ ID NO: 5, nucleotides 1575 to 1703 of SEQ ID NO:7, nucleotides 1834 to 1965 of SEQ ID NO: 9, nucleotides 1649 to 1783 of SEQ ID NO: 11, nucleotides 1697 to 1804 of SEQ ID NO: 13 or nucleotides 1400 to 1401 and/or 1511 to 1619 of SEQ ID NO:15,
   (ii) the cDNA sequence thereof, or
   (iii) the full-length complement of (i) or (ii);
(c) a catalytic domain encoded by a polynucleotide having at least 90% sequence identity to nucleotides 1370 to 1504 of SEQ ID NO: 1, nucleotides 1615 to 1629 and/or 1680 to 1784 of SEQ ID NO: 3, nucleotides 1498 to 1620 of SEQ ID NO: 5, nucleotides 1575 to 1703 of SEQ ID NO:7, nucleotides 1834 to 1965 of SEQ ID NO: 9, nucleotides 1649 to 1783 of SEQ ID NO: 11, nucleotides 1697 to 1804 of SEQ ID NO: 13 or nucleotides 1400 to 1401 and/or 1511 to 1619 of SEQ ID NO:15, or the cDNA sequence thereof;
(d) a variant of amino acids 428 to 472 of SEQ ID NO: 2, 502 to 541 of SEQ ID NO: 4, 463 to 503 of SEQ ID NO: 6, 431 to 473 of SEQ ID NO: 8, 572 to 615 of SEQ ID NO: 10, 517 to 561 of SEQ ID NO: 12, 546 to 581 of SEQ ID NO: 14 or 448 to 484 of SEQ ID NO: 16, comprising a substitution, deletion, and/or insertion at one or more (e.g., several) positions; and
(e) a fragment of the catalytic domain of (a), (b), (c), or (d) that has Berberine Bridge Enzyme Like activity.

### Embodiment 74

The polypeptide of embodiment 73, further comprising a FAD binding domain.

### Embodiment 75

An isolated polypeptide comprising a FAD binding domain operably linked to a catalytic domain, wherein the binding domain is selected from the group consisting of:
(a) a FAD binding domain having at least 60% sequence identity to amino acids 48 to 186 of SEQ ID NO: 2, 122 to 256 of SEQ ID NO: 4, 80 to 213 of SEQ ID NO: 6, 54 to 188 of SEQ ID NO: 8, 179 to 319 of SEQ ID NO: 10, 128 to 265 of SEQ ID NO: 12, 119 to 261 of SEQ ID NO: 14 or 63 to 199 of SEQ ID NO: 16;
(b) a FAD binding domain encoded by a polynucleotide that hybridizes under medium, medium-high, high, or very high stringency conditions with
(i) nucleotides 142 to 259 and/or 348 to 646 of SEQ ID NO: 1, nucleotides 424 to 509 and/or 561 to 879 of SEQ ID NO: 3, nucleotides 238 to 279 and/or 338 to 697 of SEQ ID NO: 5,
nucleotides 160 to 201, 266 to 482 and/or 548 to 693 of SEQ ID NO:7, nucleotides 590 to 1012 of SEQ ID NO: 9, nucleotides 433 to 524 and/or 574 to 895 of SEQ ID NO: 11, nucleotides 355 to 470 and/or 532 to 844 of SEQ ID NO: 13 and/or nucleotides 187 to 228 and/or 287 to 655 of SEQ ID NO:15,
(ii) the cDNA sequence thereof, or
(iii) the full-length complement of (i) or (ii);
(c) a FAD binding domain encoded by a polynucleotide having at least 90% sequence identity to nucleotides nucleotides 142 to 259 and/or 348 to 646 of SEQ ID NO: 1, nucleotides 424 to 509 and/or 561 to 879 of SEQ ID NO: 3, nucleotides 238 to 279 and/or 338 to 697 of SEQ ID NO: 5, nucleotides 160 to 201, 266 to 482 and/or 548 to 693 of SEQ ID NO:7, nucleotides 590 to 1012 of SEQ ID NO: 9, nucleotides 433 to 524 and/or 574 to 895 of SEQ ID NO: 11, nucleotides 355 to 470 and/or 532 to 844 of SEQ ID NO: 13 and/or nucleotides 187 to 228 and/or 287 to 655 of SEQ ID NO:15, or the cDNA sequence thereof;
(d) a variant of amino acids 48 to 186 of SEQ ID NO: 2, 122 to 256 of SEQ ID NO: 4, 80 to 213 of SEQ ID NO: 6, 54 to 188 of SEQ ID NO: 8, 179 to 319 of SEQ ID NO: 10, 128 to 265 of SEQ ID NO: 12, 119 to 261 of SEQ ID NO: 14 or 63 to 199 of SEQ ID NO: 16 comprising a substitution, deletion, and/or insertion at one or more (e.g., several) positions; and
(e) a fragment of the FAD binding domain of (a), (b), (c), or (d) that has binding activity.

### Embodiment 76

The polypeptide of embodiment 75, wherein the catalytic domain is obtained from a hydrolase, isomerase, ligase, lyase, oxidoreductase, or transferase, e.g., an aminopeptidase, amylase, carbohydrase, carboxypeptidase, catalase, cellobiohydrolase, cellulase, chitinase, cutinase, cyclodextrin glycosyltransferase, deoxyribonuclease, endoglucanase, esterase, alpha-galactosidase, beta-galactosidase, glucoamylase, alpha-glucosidase, beta-glucosidase, invertase, laccase, lipase, mannosidase, mutanase, oxidase, pectinolytic enzyme, peroxidase, phytase, polyphenoloxidase, proteolytic enzyme, ribonuclease, transglutaminase, xylanase, berberine bridge enzymes or beta-xylosidase.

### Embodiment 77

A composition comprising the polypeptide of any of embodiments 65-76.

### Embodiment 78

An isolated polynucleotide encoding the polypeptide of any of embodiments 65-76.

### Embodiment 79

A nucleic acid construct or expression vector comprising the polynucleotide of embodiment 78 operably linked to one or more control sequences that direct the production of the polypeptide in an expression host.

### Embodiment 80

A recombinant host cell comprising the polynucleotide of embodiment 78 operably linked to one or more control sequences that direct the production of the polypeptide.

### Embodiment 81

A method of producing the polypeptide of any of embodiments 65-76, comprising:
(a) cultivating a cell, which in its wild-type form produces the polypeptide, under conditions conducive for production of the polypeptide; and
(b) recovering the polypeptide.

### Embodiment 82

A method of producing a polypeptide having Berberine Bridge Enzyme Like activity, comprising:
(a) cultivating the host cell of embodiment 17 under conditions conducive for production of the polypeptide; and
(b) recovering the polypeptide.

### Embodiment 83

A transgenic plant, plant part or plant cell transformed with a polynucleotide encoding the polypeptide of any of embodiments 65-76.

### Embodiment 84

A method of producing a polypeptide having Berberine Bridge Enzyme Like (BBEL) activity, comprising:
(a) cultivating the transgenic plant or plant cell of embodiment 83 under conditions conducive for production of the polypeptide; and
(b) recovering the polypeptide.

### Embodiment 85

A method of producing a mutant of a parent cell, comprising inactivating a polynucleotide encoding the polypeptide of any of embodiments 65-76, which results in the mutant producing less of the polypeptide than the parent cell.

### Embodiment 86

A mutant cell produced by the method of embodiment 85.

### Embodiment 87

The mutant cell of embodiment 86, further comprising a gene encoding a native or heterologous protein.

### Embodiment 88

A method of producing a protein, comprising:
(a) cultivating the mutant cell of embodiment 86 or 87 under conditions conducive for production of the protein; and
(b) recovering the protein.

### Embodiment 89

A double-stranded inhibitory RNA (dsRNA) molecule comprising a subsequence of the polynucleotide of embodiment 78, wherein optionally the dsRNA is an siRNA or an miRNA molecule.

### Embodiment 90

The double-stranded inhibitory RNA (dsRNA) molecule of embodiment 89, which is about 15, 16, 17, 18, 19, 20, 21, 22, 23, 24, 25 or more duplex nucleotides in length.

### Embodiment 91

A method of inhibiting the expression of a polypeptide having Berberine Bridge Enzyme Like (BBEL) activity in a cell, comprising administering to the cell or expressing in the cell the double-stranded inhibitory RNA (dsRNA) molecule of embodiment 89 or 90.

### Embodiment 92

A cell produced by the method of embodiment 91.

### Embodiment 93

The cell of embodiment 92, further comprising a gene encoding a native or heterologous protein.

### Embodiment 94

A method of producing a protein, comprising:
(a) cultivating the cell of embodiment 92 or 93 under conditions conducive for production of the protein; and
(b) recovering the protein.

### Embodiment 95

An isolated polynucleotide encoding a signal peptide comprising or consisting of amino acids 1 to 20 of SEQ ID NO: 4, amino acids 1 to 20 of SEQ ID NO: 6, amino acids 1 to 13 of SEQ ID NO: 8, amino acids 1 to 20 of SEQ ID NO: 10, amino acids 1 to 19 of SEQ ID NO: 12, amino acids 1 to 18 of SEQ ID NO: 14 or amino acids 1 to 20 of SEQ ID NO: 16.

### Embodiment 96

A recombinant host cell comprising a gene encoding a protein operably linked to the polynucleotide of embodiment 95, wherein the gene is foreign to the polynucleotide encoding the signal peptide.

### Embodiment 97

A method of producing a protein, comprising:
(a) cultivating a recombinant host cell comprising a gene encoding a protein operably linked to the polynucleotide of embodiment 95 or 96, wherein the gene is foreign to the polynucleotide encoding the signal peptide, under conditions conducive for production of the protein; and
(b) recovering the protein.

1. An enzyme composition comprising a cellulolytic preparation and a Berberine Bridge Enzyme Like (BBEL) enzyme.

2. The enzyme composition of claim 1, wherein the cellulolytic preparation is derived from Trichoderma reesei, Humicola insolens or Chrysosporium lucknowense.

3. The enzyme composition of claim 1, wherein the Berberine Bridge Enzyme Like (BBEL) enzyme is derived from a strain of the genus Aspergillus, such as a strain of Aspergillus aculeatus, such as Aspergillus aculeatus CBS 172.66 or a Berberine Bridge Enzyme Like (BBEL) enzyme having at least 80% sequence identity to the mature polypeptide of any of SEQ ID NO: 6, SEQ ID NO: 8 or SEQ ID NO: 10, at least 75% sequence identity to the mature polypeptide of any of SEQ ID NO: 12 or SEQ ID NO:14, or at least 60% sequence identity to the mature polypeptide of SEQ ID NO: 16 herein or wherein the Berberine Bridge Enzyme Like (BBEL) enzyme is derived from a strain of the genus Penicillium, such as a strain of Penicillium pinophilum or an Berberine Bridge Enzyme Like (BBEL) enzyme having at least 95% sequence identity to the mature polypeptide of SEQ ID NO: 2 or at least 90% sequence identity to the mature polypeptide of SEQ ID NO: 4 herein.

4. The enzyme composition of claim 1, wherein the cellulolytic preparation comprises a GH61 polypeptide having cellulolytic enhancing activity, a beta-glucosidase, a xylanase, a beta-xylosidase, a CBHI and a CBHII.

5. A method of hydrolyzing cellulosic material, comprising subjecting the cellulosic material to a cellulolytic preparation and a Berberine Bridge Enzyme Like (BBEL) enzyme.

6. (canceled)

7. (canceled)

8. The method of claim 5, wherein the pretreated cellulosic material is pretreated by chemical pretreatment, a physical pretreatment, or a chemical pretreatment and a physical pretreatment.

9. The method of claim 5, wherein hydrolysis is carried out at a temperature between 20-70° C., such as 30-60° C., preferably 45-55° C. at a pH in the range 4-6, such as 4.5-5.5.

10. The method of claim 5, wherein the cellulosic material is present at 1-20 (w/w) % of TS, such as 2-10 (w/w) % TS, such as around 5 (w/w) % TS.

11. The method of claim 5, wherein the hydrolysis is carried out for 1-20 days, preferably between from 3-15 days.

12. The method of claim 5, wherein the cellulolytic preparation is derived from Trichoderma reesei, Humicola insolens or Chrysosporium lucknowense.

13. The method of claim 5, wherein the Berberine Bridge Enzyme Like (BBEL) enzyme is derived from a strain of the genus Aspergillus, such as a strain of Aspergillus aculeatus, such as Aspergillus aculeatus CBS 172.66 or a Berberine Bridge Enzyme Like (BBEL) having at least 80% sequence identity to the mature polypeptide of any of SEQ ID NO: 6, SEQ ID NO: 8 or SEQ ID NO: 10, at least 75% sequence identity to the mature polypeptide of any of SEQ ID NO: 12 or SEQ ID NO:14, or at least 60% sequence identity to the mature polypeptide of SEQ ID NO: 16 herein or wherein the Berberine Bridge Enzyme Like (BBEL) enzyme is derived from a strain of the genus Penicillium, such as a strain of Penicillium pinophilum or Berberine Bridge Enzyme Like (BBEL) enzyme having at least 95% sequence identity to the mature polypeptide of SEQ ID NO: 2 or at least 90% sequence identity to the mature polypeptide of SEQ ID NO: 4 herein.

14. The method of claim 5, wherein the cellulolytic preparation is added in amounts of about 0.01 to about 50.0 mg, e.g., about 1 to about 25 mg, such as about 2-10 mg, such as about 4 to about 8 mg protein per gram cellulosic material.

15. A process of producing a fermentation product from cellulosic material, comprising:
(a) hydrolyzing said cellulosic material by subjecting the material to an enzyme composition according to claim 1 or according to any one of the hydrolysis method claims in any one of claims 5-14;
(b) fermenting using a fermenting organism; and
(c) optionally recovering the fermentation product.

16. An isolated polypeptide having berberine bridge enzyme like (BBEL) enzymatic activity selected from the group consisting of:
(a) a polypeptide having at least 95% sequence identity to the mature polypeptide of SEQ ID NO: 2, at least 90% sequence identity to the mature polypeptide of SEQ ID NO: 4, at least 80% sequence identity to the mature polypeptide of any of SEQ ID NO: 6, SEQ ID NO: 8 or SEQ ID NO: 10, at least 75% sequence identity to the mature polypeptide of any of SEQ ID NO: 12 or SEQ ID NO:14, or at least 60% sequence identity to the mature polypeptide of SEQ ID NO: 16;
(b) a polypeptide encoded by a polynucleotide that hybridizes under medium, medium-high, high, or very high stringency conditions with
(i) the mature polypeptide coding sequence of any of SEQ ID NO: 1, SEQ ID NO: 3, SEQ ID NO: 5, SEQ ID NO: 7, SEQ ID NO: 9, SEQ ID NO: 11, SEQ ID NO: 13, SEQ ID NO: 15,
(ii) the cDNA sequence thereof or
(iii) the full-length complement of (i) or (ii);
(c) a polypeptide encoded by a polynucleotide having at least 95% sequence identity to the mature polypeptide coding sequence of SEQ ID NO: 1, at least 90% sequence identity to the mature polypeptide coding sequence of SEQ ID NO: 3, at least 80% sequence identity to the mature polypeptide coding sequence of any of SEQ ID NO: 5, SEQ ID NO: 7 or SEQ ID NO: 9, at least 75% sequence identity to the mature polypeptide coding sequence of any of SEQ ID NO: 11 or SEQ ID NO:13, or at least 60% sequence identity to the mature polypeptide coding sequence of SEQ ID NO: 15
(d) a variant of the mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16, comprising a substitution, deletion, and/or insertion at one or more (e.g., several) positions; and
(e) a fragment of the polypeptide of (a), (b), (c), or (d) that has Berberine Bridge Enzyme Like enzyme activity.

17. (canceled)

18. (canceled)

19. (canceled)

20. The polypeptide of claim 16, comprising or consisting of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16, or the mature polypeptide of SEQ ID NO: 2, SEQ ID NO: 4, SEQ ID NO: 6, SEQ ID NO: 8, SEQ ID NO: 10, SEQ ID NO: 12, SEQ ID NO: 14 or SEQ ID NO: 16.

21. An isolated polypeptide comprising a catalytic domain selected from the group consisting of:
(a) a catalytic domain having at least 60% sequence identity to amino acids 428 to 472 of SEQ ID NO: 2, 502 to 541 of SEQ ID NO: 4, 463 to 503 of SEQ ID NO: 6, 431 to 473 of SEQ ID NO: 8, 572 to 615 of SEQ ID NO: 10, 517 to 561 of SEQ ID NO: 12, 546 to 581 of SEQ ID NO: 14 or 448 to 484 of SEQ ID NO: 16;
(b) a catalytic domain encoded by a polynucleotide that hybridizes under medium, medium-high, high, or very high stringency conditions with
(i) nucleotides 1370 to 1504 of SEQ ID NO: 1, nucleotides 1615 to 1629 and/or 1680 to 1784 of SEQ ID NO: 3, nucleotides 1498 to 1620 of SEQ ID NO: 5, nucleotides 1575 to 1703 of SEQ ID NO:7, nucleotides 1834 to 1965 of SEQ ID NO: 9, nucleotides 1649 to 1783 of SEQ ID NO: 11, nucleotides 1697 to 1804 of SEQ ID NO: 13 or nucleotides 1400 to 1401 and/or 1511 to 1619 of SEQ ID NO:15,
(ii) the cDNA sequence thereof, or
(iii) the full-length complement of (i) or (ii);
(c) a catalytic domain encoded by a polynucleotide having at least 90% sequence identity to nucleotides 1370 to 1504 of SEQ ID NO: 1, nucleotides 1615 to 1629 and/or 1680 to 1784 of SEQ ID NO: 3, nucleotides 1498 to 1620 of SEQ ID NO: 5, nucleotides 1575 to 1703 of SEQ ID NO:7, nucleotides 1834 to 1965 of SEQ ID NO: 9, nucleotides 1649 to 1783 of SEQ ID NO: 11, nucleotides 1697 to 1804 of SEQ ID NO: 13 or nucleotides 1400 to 1401 and/or 1511 to 1619 of SEQ ID NO:15, or the cDNA sequence thereof;
(d) a variant of amino acids 428 to 472 of SEQ ID NO: 2, 502 to 541 of SEQ ID NO: 4, 463 to 503 of SEQ ID NO: 6, 431 to 473 of SEQ ID NO: 8, 572 to 615 of SEQ ID NO: 10, 517 to 561 of SEQ ID NO: 12, 546 to 581 of SEQ ID NO: 14 or 448 to 484 of SEQ ID NO: 16, comprising a substitution, deletion, and/or insertion at one or more (e.g., several) positions; and
(e) a fragment of the catalytic domain of (a), (b), (c), or (d) that has Berberine Bridge Enzyme Like activity.

22. A composition comprising the polypeptide of claim 16.

23. An isolated polynucleotide encoding the polypeptide of claim 16.

24. A recombinant host cell comprising the polynucleotide of claim 23 operably linked to one or more control sequences that direct the production of the polypeptide.

25. A method of producing a polypeptide having Berberine Bridge Enzyme Like (BBEL) activity, comprising:
(a) cultivating the host cell of claim 24 under conditions conducive for production of the polypeptide; and
(b) recovering the polypeptide.


SEQUENCE LISTING (16 sequences)

SEQ ID NO: 1 - DNA, 1513 bp, Penicillium pinophilum
atgggcaatacgacaagtgcggtagctcagcaatgcttgctggacgctgttggaaacgacgcgtctttagtcgctttcccctcagcattgctatatggatcagtcgatgtgaatccttataacctggattatccggtaaccccggccgctgtcacatttcctgaatctgcagaccaagtggccgctatcgttaaatgcgctgtggatgcagatgctaaggtgcaagcaaaaagtggcggtcatagctatgccaactatggtaggctaattttcatttctttgcatttgatgtaaaaagtatatcattttcggaacagcgtaccaatgctcatcacatcatttcacaggtcttggaggcgacgacggtgcaattgtcgttgacttgaggcatttccaacaattttcatacgacccaaccactcaatatgcgacaatcggtgccggtacattgctcggggatatcgatacaaaacttcacgatgccggcaatcgtgccatgactcacggcacctctccgcaggtcggtattggtggacatgccacaatcggagggcttggaccgactgctagacaattcggaatggcattggaccacgttgagtctgttcaggtcgttctggccaattctagcatcgtgactgcgtcgactaccgagtaccctgatattttctatgcgataaaaggtgctggagcaagcttcggcgttgtcaccgagttcaccgttcgcacagaagctgagcccggcatcgcggtccaatatcaattcacgttcaacctcggagacacaagttcacgcgccaacacgttcaaggcgtggcaacaattcatagccgatcccagcctaccgagagagttttcatgtcaacttgtcctagctgaaggtatactattgatcgagggcgagttcttcgggtccctcgaggattttgaggcccttcaactggaaagcaaattccccgccaaccaaggttacaatgtcacggtgtttgacgactggcttgctcttgttgcggcatggggtgttcaactaggtgaagatcttacgggaggaattccagcacatttctacagcaaatcgcttccatttaccaacaccagtctcatcccagatgacgttgtcgatgactttttcgagtacatcgacacagcagataaagacacgctgctatggttcattattttcgatcttgaaggaggcgcaatcagcgatgtgcccgtacatgcaacctcgtacggccaccgagacgcattgttctggttgcagtcttatgctattaaccttcttggacccgtgtccgccacgacgaagacgtttttgaatcaagtcaataatgttttcctcactggcatgcccgatgcgacttttggcgcctaccctggatacgtcgaccgagaattgacaaacggaccggaacaatactggggccctaacctggagacacttatagagatgaaatccgctgtggatcctcaggacattttccacaaccctcaaagcgttcccctgcaataa

SEQ ID NO: 2 - protein, 474 aa, Penicillium pinophilum
MGNTTSAVAQQCLLDAVGNDASLVAFPSALLYGSVDVNPYNLDYPVTPAAVTFPESADQVAAIVKCAVDADAKVQAKSGGHSYANYGLGGDDGAIVVDLRHFQQFSYDPTTQYATIGAGTLLGDIDTKLHDAGNRAMTHGTSPQVGIGGHATIGGLGPTARQFGMALDHVESVQVVLANSSIVTASTTEYPDIFYAIKGAGASFGVVTEFTVRTEAEPGIAVQYQFTFNLGDTSSRANTFKAWQQFIADPSLPREFSCQLVLAEGILLIEGEFFGSLEDFEALQLESKFPANQGYNVTVFDDWLALVAAWGVQLGEDLTGGIPAHFYSKSLPFTNTSLIPDDVVDDFFEYIDTADKDTLLWFIIFDLEGGAISDVPVHATSYGHRDALFWLQSYAINLLGPVSATTKTFLNQVNNVFLTGMPDATFGAYPGYVDRELTNGPEQYWGPNLETLIEMKSAVDPQDIFHNPQSVPLQ

SEQ ID NO: 3 - DNA, 1847 bp, Penicillium pinophilum
atgcgttggtctgcagtcatctgttcattcttgctctcagttgtccctgctaagtctgcgtcgtctatatgtcgttgttttcctggtgatacctgctggccttcggctgccgcatgggcgaagttcaaccagtctgtcgatggaactctaattgcaacggtacctttgggtactccctgccatgctccatcatacaatgccgccgagtgcgatgttcttcgcaatggatggctcctaccggaagagcagtaagtttgacttccaaaacgctgtgttgctcaccgaatttgacccgatactttccgtagctatgaatcttcatcgtcgattatggccccatggtttaccaacgagacttgcgatccattccatccagtctcgcagccttgtactttaggaaactatgttgtctattcggttaatgtttcaaagcctattcatgtttccaagacacttgcatttgcgaaacagaataacatccgtgttgtcgttcgcaacactggccacgagtgagcatctcttccccttaccttgtatttgcatagctaaccatgtcatagctacaatggcaagtccacaggtgctggtgctatcgccatctggacccataacctgaaagatcgggaagttattgactacaacagcacctactaccaaggtaaagccgtcaaactcggtgccggtgttcaaggtattgaggcgtaccagctagcagacgcaaacggccttcaggtcgttggcggagaatgtccaaccgtcggtattgctggcggttactcccaaggcggcggtcattccgctctctcttcccgctatggattgggtgctgatcaggtggtggaatgggaagttattgacggcacgggagagttcagggttgctaatcgcgagcaaaactcggatttgtactgggctctttccggtggtggtggtagcacatatggtgttgtgtggtcgatgacgtccaaagctcatgcaagcacgccagtatctggtttgaacctgacgttcacaaacgaaggcatctcccaagacacttactacaaggctgtgggtatctatcatgccaaccttgcatccatcgttgatgctggtgccatgagcgtctggtatttcaccaataccagtttcgcaatctccccgattaccgcacctaacattccccttgccgaattgatcaatctcgccaaacccttcactgatgccctgaccgagctcggcatcaaatataccacctactctcaacagttcgacacttatctagacgagtacaccggtatgcagggtgccatcgaagttgggattgcccaatacggcggctggcttatcccacgctccgtcgttgagacgaacaacgataacttgactgatgcctatcgctacattaccgaagatggagctcagtttatcggtgttggtttgaatgtttccaaggccgttgttggaaccgatatatacaatgccgttttgcctgcttggagagataccttgattgatactgtgatcaccacaccatggaactttacagctccgaagtctgatatggtcgcggaacaactcaagatgaccaacgactatatcccgcgcttgaaggctttggcttctaattcgggtgcctacatgaacgaggtacgtttacttagctttgcttctagcagtgatgactgacataaccacaggccgacttccgtcaaccggacttccaacaagcattcttcggcgataactaccaacagctgcaagccgtcaaagccaagtacgatcctgatgacttgttctacgctcttactgctgttaactcggaccaatggactgaattggaggacggccggttatgtaaagcatga

SEQ ID NO: 4 - protein, 561 aa, Penicillium pinophilum
MRWSAVICSFLLSVVPAKSASSICRCFPGDTCWPSAAAWAKFNQSVDGTLIATVPLGTPCHAPSYNAAECDVLRNGWLLPEEHYESSSSIMAPWFTNETCDPFHPVSQPCTLGNYVVYSVNVSKPIHVSKTLAFAKQNNIRVVVRNTGHDYNGKSTGAGAIAIWTHNLKDREVIDYNSTYYQGKAVKLGAGVQGIEAYQLADANGLQVVGGECPTVGIAGGYSQGGGHSALSSRYGLGADQVVEWEVIDGTGEFRVANREQNSDLYWALSGGGGSTYGVVWSMTSKAHASTPVSGLNLTFTNEGISQDTYYKAVGIYHANLASIVDAGAMSVWYFTNTSFAISPITAPNIPLAELINLAKPFTDALTELGIKYTTYSQQFDTYLDEYTGMQGAIEVGIAQYGGWLIPRSVVETNNDNLTDAYRYITEDGAQFIGVGLNVSKAVVGTDIYNAVLPAWRDTLIDTVITTPWNFTAPKSDMVAEQLKMTNDYIPRLKALASNSGAYMNEADFRQPDFQQAFFGDNYQQLQAVKAKYDPDDLFYALTAVNSDQWTELEDGRLCKA

SEQ ID NO: 5 - DNA, 1638 bp, Aspergillus aculeatus
atgtatcggttcaagctggaattcgtgacggttgtcgccacgtgggcgttgtcggcagcctgtcattccctgcctcgtgcggccgatgggtcagacggcgcttgccactcaggcgatgccgacttcaaggctctcggagccaagctgtcgagcaccgcgcaggtctattgcccgggctccagtgaattcaccaatgtaaccacccgctggtcggtgctcgaagagcccgaggtgaacattgtctttgtgccaggcacggagaatgatgttgccgagattgtatgcccgccttgatccacgcactatcaggaagatatactgacaagacgatgtgtaggtgcaatatgccaacaagaaggaaatccccttcctgacctacaatggcgtccatggggctctcacctccctgggcaagatggactacgggatagccatctacatgggccagctgagcggtgtccaggtcgccgctgatggccagaccgcgaccttcggtggcggcaccatgtccaaaattgtgacggacgagctgtgggccgccggcaagcagacggtgactggcacttgcgaatgtgtcagtctcctgggccccgctctgggaggtggccacggctggctgcagggccaccacggcctcgtcgccgaccagttcgtctcgatgaacatcgtgctggccaacggcacgctgaccaccatcgacgagacctccgacctctggtgggccgtcaagggcgcgggccacaacttcgggatcgtcacctcgctgacctcgaagatctacgacatcgagcaccgcgactgggccatcgagaccatcatcttcagcggcgaccaggtcgaggccgtctaccagacgacaaacgactacctcttgaagaacggcacgcagcccgagggtgtgatcaactggtcctactggatgaacaacgccgacgctgaccccaacaacgtacgttcccttcccatcccacttaccagacctccaaactaacccccttcaagcccgttatcgtcttttacctcatccaagaaggcgtcaccgccgtcgactccacctacagcgctcccttccacgcactcggccccatttccgtgacccccgccaacggcacctacaaagacctggccggctggaccagcatcgctgtcgactccccgccctgccagaagatgggcatggccaatccgcgcttcccgatttacctcgagacgtacgacgtggcagcccagaagcaggcatgggacgtctacgcagatgccacgcgcggcgcctccccgttcaacaactccatcttcatgttcgagggctactccgcgcagggcgtgcacgccgtcgacagcgactccagcgccttcgccttccggtccgagaacctcctcgccgccccgatgatcaattacttccccgacggggacgagctggaccagaaggccaacagcgtgggtcagcagcttcgcgagatcttattcaacgcgaccggccgctcggagatccgcgcgtatgtcaattatgcgtatggcacggagtcgccgcgggagttgtacgggagtgaggactggcggcagcagcggttgcaggcgttgaagcagaagtatgatcccaccggaaagttcagcttttatgcgcctgttgcgtga

SEQ ID NO: 6 - protein, 508 aa, Aspergillus aculeatus
MYRFKLEFVTVVATWALSAACHSLPRAADGSDGACHSGDADFKALGAKLSSTAQVYCPGSSEFTNVTTRWSVLEEPEVNIVFVPGTENDVAEIVQYANKKEIPFLTYNGVHGALTSLGKMDYGIAIYMGQLSGVQVAADGQTATFGGGTMSKIVTDELWAAGKQTVTGTCECVSLLGPALGGGHGWLQGHHGLVADQFVSMNIVLANGTLTTIDETSDLWWAVKGAGHNFGIVTSLTSKIYDIEHRDWAIETIIFSGDQVEAVYQTTNDYLLKNGTQPEGVINWSYWMNNADADPNNPVIVFYLIQEGVTAVDSTYSAPFHALGPISVTPANGTYKDLAGWTSIAVDSPPCQKMGMANPRFPIYLETYDVAAQKQAWDVYADATRGASPFNNSIFMFEGYSAQGVHAVDSDSSAFAFRSENLLAAPMINYFPDGDELDQKANSVGQQLREILFNATGRSEIRAYVNYAYGTESPRELYGSEDWRQQRLQALKQKYDPTGKFSFYAPVA

SEQ ID NO: 7 - DNA, 1709 bp, Aspergillus aculeatus
atgctcgctgtcctgtccataattgccggagcagttgcatctaatatcactgctcttctgacccatctttcctcgtccagcgaagtgttctatcccaacgatgccgactggggcgatatcatccagcgatggaccgcctgggatgagcccactttctcactggctatcaagcccggctcagtcgccgatttgcagacggttgtatgttggcgtcccaaaccccaagacctacctcactcgctgactgagtttatcttggctacaggtcaaatatgccaccagtcagaatttaccattcctggccagcggaggtggccatggatatagcacctcatttggggctcttcagcaggggcttcaaattgatatgggatcgttcgacagcatccaaatcaatgctgagaagagcacgatgaccatcggtggttccgttaaattctcacaaattatggagccccttttcaatgctggaaaggaaattcgtgtgtattcctggtatgttacaggatggttaacatggttactaaccagtgggcaatgccaatagaaaccggatcctgcccctgcgtgggcatggtaggggcaactctcggtggcggagtagggcgctatcagggcctgcacggtctgatcatcgacgcgctggagtccgtggaacttgtcactgtgaagggcgatctcatcactgtgtcggctacagagaacgcagacctattctggggcctgcgcggcgccggcttcaactacggcattgtcacctcggccacatataaggtgtatgatctcaccaacaagggcaacgtactcaatgccgacttcatgttccctgccgagcttaacgagacctacttcaagattctggctagctatgacaccctcccagcggcgttgtcattgtataccttggtgatgaacgatgctgccgcaggagtaagtcagagtctccctctcctctcttgaaactccatgattgaagtctaaccaccaccctgtctgaggtgtaatagccggtgattctcctgaacgccgtgtaccctggtcccgagcaagagggtctcgatctcctggccccgttcctcaacctcacctatctcgagcgcaacatctccatgattacctgggatgcggtcgacgccgacgccggttttggtcttgccacggccttctgcgtcaacggcgagaaacatgacatgtggtccctcggtgtccgcaccatcgacgcagccacccacatcacctactttaaccaactcgcacaattctggtcggattacccggccgcgtccggcagcacttggcaggtggaatattttcctgtccaagctgtgacggccgtggccgatgcatcgactgcctatccccatcgagaaatccgtgctcatgagtatttcctccccctcttatccttgcgcacatctgcagaaaatggcggacgcatgactgaccctgtatctatgattagaatgttctccttcaacctcaccgattcttccatcggaacatctgtcgacgactttgcccttgcagccctggagaccttcaacgcgaccagtgggttcgacgatcttcatatctacgtcagctatgcccacggaacagagggactggaccctatgtatggatccgagaaactgtcgcgcttgttggcgctgaagaaacagtgggatccccagggcttcttcagctacaacaacgggttgccgcactga

SEQ ID NO: 8 - protein, 474 aa, Aspergillus aculeatus
MLAVLSIIAGAVASNITALLTHLSSSSEVFYPNDADWGDIIQRWTAWDEPTFSLAIKPGSVADLQTVVKYATSQNLPFLASGGGHGYSTSFGALQQGLQIDMGSFDSIQINAEKSTMTIGGSVKFSQIMEPLFNAGKEIQTGSCPCVGMVGATLGGGVGRYQGLHGLIIDALESVELVTVKGDLITVSATENADLFWGLRGAGFNYGIVTSATYKVYDLTNKGNVLNADFMFPAELNETYFKILASYDTLPAALSLYTLVMNDAAAGPVILLNAVYPGPEQEGLDLLAPFLNLTYLERNISMITWDAVDADAGFGLATAFCVNGEKHDMWSLGVRTIDAATHITYFNQLAQFWSDYPAASGSTWQVEYFPVQAVTAVADASTAYPHREIRAHEMFSFNLTDSSIGTSVDDFALAALETFNATSGFDDLHIYVSYAHGTEGLDPMYGSEKLSRLLALKKQWDPQGFFSYNNGLPH

SEQ ID NO: 9 - DNA, 2037 bp, Aspergillus aculeatus
atggttccttcaacaccttcactactggccgcactgggcgtatgctcgcaggccctggccgctgtggggacagttcctgtggctgccaccacgttctcctccgccaactcaatcgccaatgcgtcgtattttgtcagtgaaacacagcagctcaccaacggtgtcttaaggacgctggggaagtatcagtccctgttcaagtttgctgacgccaacctcaccctgcccgcagtgggcacatgtaaggtttaccccggagacgctgcgtggccctcgacctcccagtggtcagtattgaaccaagcactaggcggtgctctcatcaagacagtccccctggctgcgacctgctacccagattggtctgcttactacaaccaaacagcctgtgaagagatctccgatgactggactgtgtcgtacctacagtacgttctccccctccatgggaatgtctctgaatgtaaacgctgacctaagtagtgctgcagaccctgcctcaatcatgtggcctctattcgagggtcgtagctgtctccccactgacatcgcgacttcttccacctgtaccattggtggctacagcagctatgccgtcaatgtcagcaccgtggcgcagatccagctggcagtcaatttcgctcgcaaccatgggctacgcttgacagtaaaaaacacaggccacgacttcaacggcaagtcgaccggtgctggggcattgggcatctggacccacaatctcaaggacattgaggtctatgaggaataccatctgggtaactaccgtggcccggccgtgaagctgggcgctggtgtccaggcctttgaggtttacgagaaggctcacgagctcggctacaccgtggtgggaggcgagggcaagaccgtgggtgtggccggtggatacgtgctgggcggtggccactcgcccatgtccagtgtctacggactggctgctgaccaggtcctggccctcgaagttgtgctcgctgacggtcgcttcgttactataaccgagaagtccaacccggacctcttctgggctcttcgcggcggcggcggtggtaagcatatatccccggattgccacccatccctcttcactgttcatacttaacacaattcatagggacctacggtgtcgtgacatccattatctcgcgccttcaccccaagattcccgtcaccgtctccaccttttccttcatgacttccactaacgtgactaccgacaccttctgggccggcgtggatgcgtacatgaagcgcttcccgaccaacgccgacgcgggcacgtacgcctacttctggatcatcaagctcggcgatgagagcttcgaattcctgatgaatccctactttgcggtgaactacacggtggccgagtttaaccagctgatggccccgtggttcgccgacctggaccggctgggcattacctacaccccgaacacgacctactacgacaacttttacgacgcctgggaagcgggtttcccccttgaaaccatcggcagtgtgacgatgatgaccggctcacgcctcatcccgcgcgagaacttcgtcaactcaacgctcttcaatcagaccactgccgccttccgttcgaccgtctctgcgggctactcgctgctggccttcaacctcaaggccgagcttcccaccggttacccagagagcgcggccaacccggccttccggaccacgctgatgcacgccatcacctcaaccagctgggacagcaacgcgaccaatgcggagattctgaagaccatggataccttgacgtatgatgtgctgggcgagtggcgcaagacatgcccggactccggggcttacatgtccgaggcagacatccaggagccggatttccagcaggcattctacggaaatttctacgcacgcctctatgctctcaagcagcagtgggatcccacgggtctgttctatgcgcctacggccgtggggagcgaggattgggaggtgagaagcttggatgggttaccagatcagaatgggcggttgtgccgcatttaa

SEQ ID NO: 10 - protein, 638 aa, Aspergillus aculeatus
MVPSTPSLLAALGVCSQALAAVGTVPVAATTFSSANSIANASYFVSETQQLTNGVLRTLGKYQSLFKFADANLTLPAVGTCKVYPGDAAWPSTSQWSVLNQALGGALIKTVPLAATCYPDWSAYYNQTACEEISDDWTVSYLHAADPASIMWPLFEGRSCLPTDIATSSTCTIGGYSSYAVNVSTVAQIQLAVNFARNHGLRLTVKNTGHDFNGKSTGAGALGIWTHNLKDIEVYEEYHLGNYRGPAVKLGAGVQAFEVYEKAHELGYTVVGGEGKTVGVAGGYVLGGGHSPMSSVYGLAADQVLALEVVLADGRFVTITEKSNPDLFWALRGGGGGTYGVVTSIISRLHPKIPVTVSTFSFMTSTNVTTDTFWAGVDAYMKRFPTNADAGTYAYFWIIKLGDESFEFLMNPYFAVNYTVAEFNQLMAPWFADLDRLGITYTPNTTYYDNFYDAWEAGFPLETIGSVTMMTGSRLIPRENFVNSTLFNQTTAAFRSTVSAGYSLLAFNLKAELPTGYPESAANPAFRTTLMHAITSTSWDSNATNAEILKTMDTLTYDVLGEWRKTCPDSGAYMSEADIQEPDFQQAFYGNFYARLYALKQQWDPTGLFYAPTAVGSEDWEVRSLDGLPDQNGRLCRI

SEQ ID NO: 11 - DNA, 1834 bp, Aspergillus aculeatus
atgcaacccatactgtggactgtgttactcggtttggctgctgggcgcagtgtaaccctccctggttctgcttccaatgcctcctgccgcgccattcctggggatccttcttggccctcgctcgaaaaatggcacgccctcaataagacggttcacggccgattgattgccacgatccccctgcccagcgtctgccacagcgagccgttcggtaactacaatgcggatgcctgcagtgccctcaaaaccacatggctggacgatacaaccttgtcggtactccagtctgcatgtataccatgttgcaactaacctatgcctagtctgaaccatccagcagaggtcatgaatccctacgtacagaactatacctgtgtccccttcactccagcgtcacaaccatgcattctaggcaattatgccagctattccattaatgtcaccggcgccgacgatgtgattgctggtgctgccttcgcacgagataatgacatccggctggtgatcaagaataccggtcatgagtacggccccaatttccatgttcgtgaagactgtatctgacccaaccagtttcatgggcaaatccagtggaacaggcgccctctccctgtggacgcacaacctcaatacgacagacatcctctcctcatactcgagtgccgactataccgggccagccgctcgactgggcgccggggtgatcagcgggcaagtctactccatcctaggggcagcgggctaccgcatcctgggtgggacatgccccaccgtcggcctggctgggggctacacctcagggggaggacactcgctgcttaacggcgcctacgggatgggcgccgacgcggtgctggaatgggaggtcatcaccgcgcagggggagcacctcgtcgcaacgccgtacaacaacaccgacctatactgggcgctcagcggtgggggaccggggaccttcgctgtggtgctcagcatgacgaccaagatattcatcgatggacccataggcagcggtctgctgttcttcaacatctctgccactaacccagagccctactggtccgccatctccgatctgtggcagttcctgccccaattcgtcgacgttggccccaacacgtgggacttcgcactgacggccaccgggttcgaagcctacgccatcaccgtgccagacaaaaacggcacacaagtgcaggatttgctaaagccgtttctcacccgcctcacccagcgggacatcccctactccttcacgcccaccacctccccgaactaccacgactacttcgccagccgctttggcccgggcatcgccggcgccggccccgccacggtgcagttgacatcgcgcctcattccccgcgccggggtgcagaacatcacgcagaaccaagccatcgtcgccgccttgcgcgccgtcgtcgagaccgacccagacctctcgctgggatgccacgccctcaacgtgaaggacatcccgcacccagacaacgccgtcctccccgcctggcgcgacgccattgctacgtgcaacatcgtcagctactgggattggaatttatccccaatacaaatgcaggcccggaaggcgcagctggtcgacacgctgatcccggggctggagcgggcgacgcccggtgcggggacgtatctgaatgagatcgatgcgcagtggaagggaaattgggaggtggagctgtatgggaggaattatccacggttgcttgaggtcaaggagaagtatgatccagcgcatgtgttttatgcgaagacggcggtggggagtgcagcgtgggaggtcgattccagagggaggttgtgtagagtttag

SEQ ID NO: 12 - protein, 577 aa, Aspergillus aculeatus
MQPILWTVLLGLAAGRSVTLPGSASNASCRAIPGDPSWPSLEKWHALNKTVHGRLIATIPLPSVCHSEPFGNYNADACSALKTTWLDDTTFLNHPAEVMNPYVQNYTCVPFTPASQPCILGNYASYSINVTGADDVIAGAAFARDNDIRLVIKNTGHDFMGKSSGTGALSLWTHNLNTTDILSSYSSADYTGPAARLGAGVISGQVYSILGAAGYRILGGTCPTVGLAGGYTSGGGHSLLNGAYGMGADAVLEWEVITAQGEHLVATPYNNTDLYWALSGGGPGTFAVVLSMTTKIFIDGPIGSGLLFFNISATNPEPYWSAISDLWQFLPQFVDVGPNTWDFALTATGFEAYAITVPDKNGTQVQDLLKPFLTRLTQRDIPYSFTPTTSPNYHDYFASRFGPGIAGAGPATVQLTSRLIPRAGVQNITQNQAIVAALRAVVETDPDLSLGCHALNVKDIPHPDNAVLPAWRDAIATCNIVSYWDWNLSPIQMQARKAQLVDTLIPGLERATPGAGTYLNEIDAQWKGNWEVELYGRNYPRLLEVKEKYDPAHVFYAKTAVGSAAWEVDSRGRLCRV

SEQ ID NO: 13 - DNA, 1885 bp, Aspergillus aculeatus
atgctacccctgatcttctggtgcgcgctttgccttaccacgcctgtggtggcaggcaaatgcaaaaacacccctcttgatgtgacatggccctctgacacggattggtcgtctttaaatgcgtccattggcggtcatctgcttcgcacagttcctgctgctgcttcctgctggcctaacggccacttggagtcctccatctcatgcaatacagttcagtcacagtggaccaacgcgctctggcatgcaagtcagccagactccattgactaccctatctacgcgaacaattcctgtctaccgccaaacgtgacgggttatgtacacgatctgggctgtacaactgggggactgccagcatatattgtcaacgctacaacggagactcagattgcgacggcaatgaagtgggccgccgagcgcagcattcgcatcgtcattaaaggcactgggcatgatttgaatgggcggtaaggcaaacggttcgcctcgcgtcacacgtcaagcgctaatatatgtttgcattgttaggtcaagtggtgcctttgctctgtcgatctggactcaccatctccgcaagttacagcgcgatatctcctggaaacacccaaccaagaatatcacggaggatgtatacatcgttggcagcggccagcagtggggtaatgtgcttaattacgcgctcgctcagggccgagtagtcaccaccggacaagaccccagcgtcggacttggtggctacattcaagggggtggacacggtccattgtcacggacctacggtcttgctgctagtcacgtactgcagatgcgcgtcgtcaccacccagggagagatacttgtagccaacgatgcacaacaccaggacctgttctgggcgctgcgcggtggcgggccgggtctgtatggtctggtcacagaatatgtgctgcgacatcatccggctccgagcaacgtgactatgggaaacctcttgatcaccccaaaagcggaccgcaacaatctcagcgcggaggcatcgtggaaagcagccgtgtcgtacctccaggcgcttccggagctgatggacgccggtctagcaggggcatgtatgatgtcaactgccgaggcagcgatgcggttcggctccctctctgaagccacgaccggcgtagtcatctcgcaagcgttctggtccttcaattcaactccatccgccatggaagccttggtccgtgcggtcctcactcgcattggacgtgactcgactccaaatagcggtagccctgcagtgaatatcacgttcagcacttccaacgttgcgaactacacctccttcatttccgccatctctggtagcaacgcagccggcggccagagcgtcgtatcaagccgccttctcggccgccgcgagctcctcggcacgtcgcgcacagacatgaccagctatctcaaaactgcgctgcaagcgcagaacgcgaccgcgggaacctttgcgacgatcggcttacaaggcggaccgggagtgcaacagacccctccaggggactggggtgctctgctaccggcatggcgcacggcgtacctgcatttcattgccaacggagcgacagtcgactccgtcgctgcgggctctccgaagcgagcgctgcaagacgccgcgcgatggtatgaggccaaggagcgcatgtggcagctgtgggcgccggagtcgggggcgtacatgaacgaggctaatccattcgatcccgagttcaagcgtgacttctatggggcgaactatgaccggttggtgcaggtcaaggccaagtacgatccgactgagagcttgtttgttctggctggtgtgggaagcgatcggtgggattatgatctagatagtggtcgactttgcagggtggattaa

SEQ ID NO: 14 - protein, 607 aa, Aspergillus aculeatus
MLPLIFWCALCLTTPVVAGKCKNTPLDVTWPSDTDWSSLNASIGGHLLRTVPAAASCWPNGHLESSISCNTVQSQWTNALWHASQPDSIDYPIYANNSCLPPNVTGYVHDLGCTTGGLPAYIVNATTETQIATAMKWAAERSIRIVIKGTGHDLNGRSSGAFALSIWTHHLRKLQRDISWKHPTKNITEDVYIVGSGQQWGNVLNYALAQGRVVTTGQDPSVGLGGYIQGGGHGPLSRTYGLAASHVLQMRVVTTQGEILVANDAQHQDLFWALRGGGPGLYGLVTEYVLRHHPAPSNVTMGNLLITPKADRNNLSAEASWKAAVSYLQALPELMDAGLAGACMMSTAEAAMRFGSLSEATTGVVISQAFWSFNSTPSAMEALVRAVLTRIGRDSTPNSGSPAVNITFSTSNVANYTSFISAISGSNAAGGQSVVSSRLLGRRELLGTSRTDMTSYLKTALQAQNATAGTFATIGLQGGPGVQQTPPGDWGALLPAWRTAYLHFIANGATVDSVAAGSPKRALQDAARWYEAKERMWQLWAPESGAYMNEANPFDPEFKRDFYGANYDRLVQVKAKYDPTESLFVLAGVGSDRWDYDLDSGRLCRVD

SEQ ID NO: 15 - DNA, 1640 bp, Aspergillus aculeatus
atgagggttccgtcagcattggggcttgctgccttggcaggtccggccgtggttgaggcgtggacgaagtacaccgcgtttgagacgtgcttgcagccacggctctcgtccacggccacgatcctcaatgcctcctcgacggtcgtcgccccgcgatggacggagtatgacgagcccgtgacgaaccacgtcgtcaacgtggcgacagaagcggacgtgcaagccactgtaccctcacccgtcccttatcccttagtattgggctgaccccacaccacaaacacaggtcttctgcgccaacacccacgacatccccttcctcgcccaagccggcggcaacgcctggagcaccacctggaccctaaccaacgcgaccaccccctccatcctcatcaacctccgcgccctcaacgccatcaccttctccccgaaccgcacaaccgtcaccgtgcaaggcggcaccctgatctccgagctcctgaccgccgcctacgcacacgacgcacaggtggtgaccgcgaactgcgactgcgtcggggtgatgggcgccctgctcgggggcgggtacagccgcatgatgggggaaagggggtacatgatcgacaacgtcctctcactaaacgtcgtgctagcaaccgggacgcgggtgacggtgacggcgcgctcgcacccggacctgtggtgggcgctccgcggcgccggcgccaacttcggggtggtcacctcggcggtcttccgctcatacccgaccccacgaaccgacaaccacgcctggctgggcagcctcgtcttcagcgaggacaagctcgaagcgttggttggggcgatcaacgacctcccgctgaccccccagatggccatcttcctttacttcgccaccaccggcgccccggactacacgccggccatcatcgcgttcccgttctacctcgcccgcgcgggggccaacacgacgacggaggaagcccgcgccgcgttccgcacgatcctcgcgctgggcccgctctccgacacgaccgcctggacgccctacgaccgggtcaacgcgggcagcggctcgttctgcgccgtcggtggccgcaaggtcagtctggggtccgctgcgcggaccctcgacccggccgcgtggcgcgcgatctggacctcgttcacgcggtgggtggcgacggccggcggcgaccaggtgggcaactcgaccgtcctgatggaggcgtacagtctggggcgggcgagtgccgtcgccacggacagctcggcgtacgcgtggcgcgcgtccagtcggttcaataccgtcgccatcgcttggtatcaggatcccgcgctggatgcgtcggcgaggcaatgggcaggggatgtgcgtgcggtgtggagggagacgagtgggttgagtggaaattctacgtgagtttgcctctttcttcggctctgcgatggttggttggggagggtgcatgatgggcgtgctcgatgcaaggtgagctgtgctgactgggcttttcttgcgggttaggtacatcaatttcgcgtttggggatgagagcctcgaggatatttatggggataatctggagaggttgcggttgttgaagaaggtctatgatccccaaaatcgatttaatcagttctttccattggcataa

SEQ ID NO: 16 - protein, 490 aa, Aspergillus aculeatus
MRVPSALGLAALAGPAVVEAWTKYTAFETCLQPRLSSTATILNASSTVVAPRWTEYDEPVTNHVVNVATEADVQATVFCANTHDIPFLAQAGGNAWSTTWTLTNATTPSILINLRALNAITFSPNRTTVTVQGGTLISELLTAAYAHDAQVVTANCDCVGVMGALLGGGYSRMMGERGYMIDNVLSLNVVLATGTRVTVTARSHPDLWWALRGAGANFGVVTSAVFRSYPTPRTDNHAWLGSLVFSEDKLEALVGAINDLPLTPQMAIFLYFATTGAPDYTPAIIAFPFYLARAGANTTTEEARAAFRTILALGPLSDTTAWTPYDRVNAGSGSFCAVGGRKVSLGSAARTLDPAAWRAIWTSFTRWVATAGGDQVGNSTVLMEAYSLGRASAVATDSSAYAWRASSRFNTVAIAWYQDPALDASARQWAGDVRAVWRETSGLSGNSTYINFAFGDESLEDIYGDNLERLRLLKKVYDPQNRFNQFFPLA